SEQUENCE LISTING (39 sequences)

SEQ ID NO: 1 - DNA, 4018 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 2 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - DNA, 3982 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 4 - protein, 1120 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 5 - DNA, 265 bp, Homo sapiens
gttcgtgctgaaggcctgtatcctaggctacacactgaggactctgttcctcccctttccgcctaggggaaagtccccggacctcgggcagagagtgccacgtgcatacgcacgtagacattccccgcttcccactccaaagtccgccaagaagcgtatcccgctgagcggcgtggcgcgggggcgtcatccgtcagctccctctagttacgcaggcagtgcgtgtccgcgcaccaaccacacggggctcattctcagcgcggct

SEQ ID NO: 6 - DNA, 24 bp, Artificial
  primer
tctaaccctaactgagaagggcgt

SEQ ID NO: 7 - DNA, 24 bp, Artificial
  primer
tgctctagaatgaacggtggaagg

SEQ ID NO: 8 - DNA, 19 bp, Artificial
  primer
aattgggttatgaggtccc

SEQ ID NO: 9 - DNA, 18 bp, Artificial
  primer
tgagaatgagccccgtgt

SEQ ID NO: 10 - DNA, 20 bp, Artificial
  primer
gtcactccactcccatgtcc

SEQ ID NO: 11 - DNA, 19 bp, Artificial
  primer
aattgggttatgaggtccc

SEQ ID NO: 12 - DNA, 21 bp, Artificial
  primer
caagagatggccacggctgct

SEQ ID NO: 13 - DNA, 21 bp, Artificial
  primer
tccttctgcatcctgtcggca

SEQ ID NO: 14 - DNA, 12 bp, Artificial
  primer
gatgtgtgcgtt

SEQ ID NO: 15 - DNA, 25 bp, Artificial
  primer
agtttctgagaatcattctgtctag

SEQ ID NO: 16 - RNA, 21 nt, Artificial
  siRNA
gccaagaagcguaucccgcuu

SEQ ID NO: 17 - RNA, 21 nt, Artificial
  siRNA
ccaagaagcguaucccgcuaa

SEQ ID NO: 18 - DNA, 25 bp, Artificial
  primer
gcgaattcctcccctttccgcctag

SEQ ID NO: 19 - DNA, 26 bp, Artificial
  primer
gcgaattcgcacgtagacattccccg

SEQ ID NO: 20 - DNA, 53 bp, Artificial
  short hairpin RNA
ggaagacagtggtgaacttccctcgagggaagttcaccactgtcttccttttt

SEQ ID NO: 21 - DNA, 58 bp, Artificial
  short hairpin RNA
aattcaaaaaggaagacagtggtgaacttccctcgagggaagttcaccactgtcttcc

SEQ ID NO: 22 - DNA, 53 bp, Artificial
  short hairpin RNA
ggaacaccaagaagttcatctctcgagagatgaacttcttggtgttccttttt

SEQ ID NO: 23 - DNA, 58 bp, Artificial
  short hairpin RNA
aattcaaaaaggaacaccaagaagttcatctctcgagagatgaacttcttggtgttcc

SEQ ID NO: 24 - DNA, 53 bp, Artificial
  short hairpin RNA
gcagagagtgccacgtgcattcaagagatgcacgtggcactctctgctttttg

SEQ ID NO: 25 - DNA, 57 bp, Artificial
  short hairpin RNA
aattcaaaaagcagagagtgccacgtgcatctcttgaatgcacgtggcactctctgc

SEQ ID NO: 26 - DNA, 54 bp, Artificial
  short hairpin RNA
gcctgtatcctaggctacacactcgagtgtgtagcctaggatacaggctttttg

SEQ ID NO: 27 - DNA, 58 bp, Artificial
  short hairpin RNA
aattcaaaaagcctgtatcctaggctacacactcgagtgtgtagcctaggatacaggc

SEQ ID NO: 28 - DNA, 54 bp, Artificial
  short hairpin RNA
gctcgaaatcttacgcaaatactcgagtatttgcgtaagatttcgagctttttg

SEQ ID NO: 29 - DNA, 58 bp, Artificial
  short hairpin RNA
aattcaaaaagctcgaaatcttacgcaaatactcgagtatttgcgtaagatttcgagc

SEQ ID NO: 30 - DNA, 54 bp, Artificial
  short hairpin RNA
ccacacatcttcaagacttaactcgagttaagtcttgaagatgtgtggtttttg

SEQ ID NO: 31 - DNA, 58 bp, Artificial
  short hairpin RNA
aattcaaaaaccacacatcttcaagacttaactcgagttaagtcttgaagatgtgtgg

SEQ ID NO: 32 - DNA, 54 bp, Artificial
  short hairpin RNA
ttgtctaaccctaactgagaactcgagttctcagttagggttagacaatttttg

SEQ ID NO: 33 - DNA, 58 bp, Artificial
  short hairpin RNA
aattcaaaaattgtctaaccctaactgagaactcgagttctcagttagggttagacaa

SEQ ID NO: 34 - DNA, 57 bp, Artificial
  short hairpin RNA
cgcaagctgaccctgagttcattcaagagatgaacttcagggtcagcttgctttttg

SEQ ID NO: 35 - DNA, 66 bp, Artificial
  short hairpin RNA
aattcaaaaagcaagctgaccctgaagttcatctcttgaatgaacttcagggtcagcttgcgggcc

SEQ ID NO: 36 - DNA, 20 bp, Artificial
  primer
gagagtgccacgtgcatacg

SEQ ID NO: 37 - DNA, 20 bp, Artificial
  primer
ctcagcgggatacgcttctt

SEQ ID NO: 38 - DNA, 15 bp, Artificial
  probe
acgtagacattcccc

SEQ ID NO: 39 - DNA, 22 bp, Artificial
  probe
cgccaatatttacgtgctgcta

(19) United States
(12) Patent Application Publication
(10) Pub. No.: US 2011/0243910 A1
(43) Pub. Date: Oct. 6, 2011

(54) MAMMALIAN RNA DEPENDENT RNA POLYMERASE

(75) Inventors: William Hahn, Boston, MA (US); Kenkichi Masutomi, Tokyo (JP); Yoshiko Maida, Tokyo (JP); Yoshihide Hayashizaki, Tokyo (JP); Timo Lassmann, Tokyo (JP)

(73) Assignees: JAPAN HEALTH SCIENCES FOUNDATION, Tokyo (JP); RIKEN, Saitama (JP); DANA-FARBER CANCER INSTITUTE, INC., Boston, MA (US)

(21) Appl. No.: 13/058,970

(22) PCT Filed: Jul. 7, 2009
(86) PCT No.: PCT/JP2009/062840

§ 371 Date: Jun. 15, 2011

(30) Foreign Application Priority Data
Aug. 12, 2008 (US) 61188743

(51) Int. Cl. A61K 38/45; C12N 9/96; G01N 33/573; C12N 5/071; C12Q 1/68; C12M 1/40; A61P 19/02; A61P 35/00; A61P 35/02; A61P 11/06; A61P 17/14; A61P 7/06; A61P 3/00; C07K 14/00

(52) U.S. Cl. 424/94.3; 435/188; 435/6.1; 435/7.8; 435/375; 435/6.19; 435/287.1; 424/94.5; 530/358

(57) ABSTRACT

The invention provides compositions comprising a TERT-RMRP or TERT-RNA complex and methods of treating subjects with genetic diseases in which gene silencing is either increased by administering the compositions of the invention or decreased by administering an inhibitor of the RNA-dependent RNA polymerase (RdRP) activity of these compositions. Moreover, the invention provides methods of screening for agonists and antagonists of RdRP activity and TERT-RMRP complex formation. Finally, the invention provides a method of identifying a RNA molecule that forms a complex with a TERT polypeptide and has RdRP activity.

### STATEMENT OF GOVERNMENT SUPPORT

This invention was made with U.S. Government support under National Institutes of Health ant ROI AG23145. The U.S. Government has certain rights in the invention. The invention was made with Japanese Government support under the Japan Science and Technology Agency grant PRESTO, under the Ministry of Education, Culture, Sports, Science and Technology grant of Grant-in-Aid for Young Scientists (A) 19689010, under the Ministry of Health, Labor of grant of the Third-Term Comprehensive Control Research for Cancer, under the Ministry of Education, Culture, Sports, Science and Technology grant of Research Grant for RIKEN Omics Science Center, under the Ministry of Education, Culture, Sports, Science and Technology grant of Grant of the Genome Network Project, under RIKEN grant of the Strategic Programs for R&D and under RIKEN grant of Grant for the RIKEN Frontier Research System, Functional RNA research program.

### TECHNICAL FIELD

This invention relates generally to the fields of molecular biology and RNA-mediated gene silencing.

### BACKGROUND ART

An RNA-dependent RNA polymerase (RDRP, RdRP, or RdRP), or RNA replicase, is an enzyme that catalyzes the replication of RNA from an RNA template. This is in contrast to a typical RNA polymerase, which catalyzes the transcription of RNA from a DNA template. Viral RDRPs were discovered in the early 1960s from studies on positive-stranded RNA virus such as mengovirus and polio virus when it was observed that these viruses were not sensitive to actinomycin D, a drug that inhibits cellular DNA directed RNA synthesis. This lack of sensitivity suggested that there was a virus specific enzyme that could copy RNA from an RNA template and not from a DNA template. The most famous example of RDRP is the polio virus RDRP and hepatitis C virus (HCV) RdRp.

### SUMMARY OF INVENTION

RdRPs have been identified in some eukaryotic organisms, such as plants, yeast, fungi, and C. elegans, with the most studied examples coming from Arabidopsis. However, the present invention is the first report of RdRP activity in a mammalian cell. Furthermore, the instant invention provides compositions containing polypeptides and polypeptide/RNA complexes that have RdRP activity as well as methods of screening for and identifying additional mammalian RdRPs. Because it is predicted that RdRP activity is required to produce siRNAs and to remodel chromatin structure even within mammalian cells, compositions and methods of the invention are used to manipulate gene expression as a means to treat disease. The compositions and methods of the invention have broad clinical appeal. The mechanism discovered by this invention will significantly impact the way that gene therapy is accomplished in the future. Manipulation of RdRP activity within mammalian cells is a powerful and precise tool. RdRP activity is targeted within specific cell populations and placed under the control of inducible activators or inhibitors. Furthermore, the overexpression of particular RNA molecules that either bind to TERT subunits or serve as templates of the RdRP complex drive production of specific siRNA molecules. Finally, agonist, antagonist, or inverse agonist compounds are used to activate, inhibit, or nullify the RdRP activity of a cell or tissue.

The invention provides a complex comprising a telomerase catalytic subunit (TERT) polypeptide or fragment thereof and a RNA component of the mitochondrial RNA processing endoribonuclease (RMRP). In one aspect of the invention, the TERT polypeptide of this complex is mammalian, e.g., human, murine, dog, cat, rat, rabbit, horse, cow, pig, sheep, goat, and primate. In another aspect of the invention, this complex has RNA dependent RNA polymerase (RdRP) activity.

Alternatively, or in addition, the invention provides a complex comprising a telomerase catalytic subunit (TERT) polypeptide and a mammalian RNA, wherein said complex has RNA dependent RNA polymerase activity.

The invention encompasses compositions which include the complexes described above. Furthermore, compositions of the invention include any pharmaceutically acceptable compound which improves one or more pharmaceutical or clinical aspect(s) of the composition.

The invention provides a method for identifying an antagonist/inhibitor of the activity of a complex of comprising a telomerase catalytic subunit (TERT) polypeptide or fragment thereof and a RNA component of the mitochondrial RNA processing endoribonuclease (RMRP) including the steps of (a) contacting the complex with a test compound; and (b) determining whether the complex has RNA dependent RNA polymerase (RdRP) activity; wherein a decrease of RdRP activity in the presence of the test compound compared to the absence of the test compound indicates that the compound is an antagonist/inhibitor of the activity of the complex.

The invention further provides a method for identifying an agonist of the activity of a complex of comprising a telomerase catalytic subunit (PERT) polypeptide or fragment thereof and a RNA component of the mitochondrial RNA processing endoribonuclease (RMRP) including the steps of (a) contacting the complex with a test compound; and (b) determining whether the complex has RNA dependent RNA polymerase (RdRP) activity; wherein an increase of RdRP activity in the presence of the test compound compared to the absence of the test compound indicates that the compound is an agonist of the activity of the complex.

The invention provides a method for identifying an enhancer of the TERT-RMRP interaction including the steps of (a) bringing into contact a TERT protein, a RMRP and a test compound under conditions where the TERT protein and the RMRP, in the absence of compound, are capable of forming a complex; and (b) determining the amount of complex formation; wherein an increase in the amount of complex formation in the presence of the test compound compared to the absence of the test compound indicates that the compound is an enhancer of the TERT-RMRP interaction.

The invention provides a method for identifying an inhibitor of the TERT-RMRP interaction including the steps of (a) bringing into contact a TERT protein, a RMRP and a test compound under conditions where the TERT protein and the RMRP, in the absence of compound, are capable of forming a complex; and (b) determining the amount of complex formation; wherein a decrease in the amount of complex formation in the presence of the test compound compared to the absence of the test compound indicates that the compound is an inhibitor of the TERT-RMRP interaction. Also provided by the invention are the agonist, antagonists, enhancers, and inhibitors identified by the methods of the invention. In certain embodiments the agonist, antagonists, enhancers, and inhibitors identified by the methods is drug or a diagnostic drug for in vivo or in vitro use for in post-translational gene silencing or chromatin based gene silencing. The invention provides a method of increasing gene silencing in a cell comprising overexpressing in the cell: (a) a telomerase catalytic subunit (TERT) polypeptide; (b) a RNA component of the mitochondrial RNA processing endoribonuclease (RMRP); or (c) both.

The invention provides a method of decreasing gene silencing in a cell comprising inhibiting or decreasing the expression in the cell of: (a) a telomerase catalytic subunit (TERT) polypeptide; (b) a RNA component of the mitochondrial RNA processing endoribonuclease (RMRP); or (c) both.

The invention provides a method of treating a disease which is caused by undesired or overexpression of a gene comprising administering to a subject in need thereof a composition comprising a TERT complex of the invention or a TERT polypeptide.

The invention provides a method of treating a disease which is caused by inappropriate deactivation of a gene necessary for cell survival comprising administering to a subject in need thereof and inhibitor of the RNA polymerase (RdRP) activity of a composition comprising a TERT complex of the invention or a TERT polypeptide.

The invention provides a method of identifying an RNA molecule that forms a complex with a telomerase catalytic subunit (TERT) polypeptide wherein said has RNA polymerase (RdRP) activity including the steps of (a) contacting the TERT polypeptide with a test RNA molecule to form a complex and (b) identifying a complex that has RdRP activity.

Also included in the invention of a device or instrument for the performance of the claimed methods.

The invention further provides a method of treating or diagnosing a disease which is caused by the altered expression or function of an RMRP comprising administering to a subject in need thereof the composition of claim 6 or a TERT polypeptide. Alternatively, or in addition, the invention provides a method of treating or diagnosing a disease which is caused by the altered expression or function of an RMRP comprising administering to a subject in need thereof an inhibitor of the RdRP activity of the composition of claim 6 or a TERT polypeptide. An exemplary disease that is caused by the altered expression or function of an RMRP is dwarfism, an immunodeficiency syndrome, asthma, atopy, an autoimmune disease, systemic lupus, erythematosus, rheumatoid arthritis, alopecia, aplastic anemia, lymphoma, leukemia or a solid cancer. Contemplated diseases are not limited to the preceeding examples. All conditions, disorders, or diseases which direct or indirect consequence or result of the altered expression or function/activity of an RMRP are encompassed by the invention.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF DRAWINGS

FIG. 1A is an Electrogram (left panel), where the red line represents RNAs recovered from control samples and the blue line represents RNAs recovered from TAP-hTERT immune complexes or as a simulated gel (right panel). Loading control indicates an internal control from the manufacturer to confirm that each sample were adequately prepared and subjected to the analysis. Ribonucleoprotein complexes were affinity purified from HeLa—S cells expressing TAP-hTERT or a control vector. RNAs were isolated from the TAP-hTERT complex and analyzed using a BIORAD Experion analyzer, a capillary electrophoresis device.

FIG. 1B is a photograph of gel electrophoresis in which RNA species associated with TAP-hTERT complexes that were isolated and subjected to RT-PCR with primers specific for the indicated RNA, are separated by size. The panel labeled RT (−) shows results obtained in the absence of reverse transcriptase (RT). Bottom panel shows the levels of TAP-hTERT in the immune complexes.

FIG. 1C is a photograph of gel electrophoresis in which hTERT complexes from 293T and HeLa cells that were purified by immunoprecipitation with an anti-hTERT antibody (Rockland) and associated RNA and subjected to RT-PCR with the indicated primers, are separated by size.

FIG. 1D is a photograph of gel electrophoresis in which RNAs purified from hTERT complexes isolated from HeLa—S cells expressing TAP-hTERT or a control vector or 293T cells and subjected to Northern blotting with the indicated probes, are separated by size.

FIG. 1E is a schematic diagram of hTERT and the deletion mutants created to map the binding site of RMRP to hTERT and a photograph of a gel electrophoresis. Conserved telomerase-specific motifs are represented by boxes. Schematic presentation of full-length FLAG epitope tagged hTERT and truncation mutants. FLAG-tagged hTERT proteins were transiently expressed in 293T cells and immune complexes were isolated using anti-FLAG-M2 antibody conjugated to agarose beads. Immune complexes were either subjected to SDS-PAGE followed by the detection by immunoblotting with the FLAG-M2 antibody (upper panel) or associated RNAs were recovered and then subjected to RT-PCR (lower panel). Positive control indicates RT-PCR products of RMRP from a total RNA to demonstrate the correct position of the product.

FIG. 2 is an agarose gel image of hTERT-associated RNAs. Isolation of hTERT-associated RNAs was accomplished using tandem affinity peptide (TAP) purification. RNP complexes were affinity purified from HeLa—S cells expressing TAP-hTERT and a control vector. RNAs were isolated from the TAP-hTERT complex and analyzed using an agarose gel. The small amounts of RNA purified from these immune complexes were difficult to visualize using this approach but were more easily resolved using an Experion device (Bio-Rad Laboratories, Inc. CA, USA) (FIG. 1A).

FIG. 3A is a photograph of gel electrophoresis in which the products of a telomerase assay performed with recombinant hTERT expressed in rabbit reticulocyte lysates in the presence of hTERC or RMRP are separated by size. TRAP assays were used to detect reconstituted telomere specific reverse transcriptase activity. Samples that were treated with RNase are indicated with a (+).

FIG. 3B is a pair of photographs showing that purified GST-hTERT-HA was fractionated by 8% SDS-PAGE and stained with Coomassie brilliant blue (CBB) or detected by immunoblotting with an anti-HA mAb (HA-11). GST was fused to the aminoterminal end of hTERT and a C-terminal HA epitope tag was added to form GST-hTERT-HA.

FIG. 3C is a schematic diagram depicting the predicted RNA products produced by RdRP or terminal transferase (TT) activity. RdRP products were synthesized from 2 different primers, from the de novo synthesized primer or from 3′ fold-back formation primer (back-priming). Terminal transferase (TT) activity incorporates ³²P-UTP at the 3′ end of the RNA template in template and primer independent manner. Those 3 different products can be discriminated by RNase T1 treatment.

FIG. 3D is a photograph of gel electrophoresis showing the separation by size of RNA products produced by the RdRP activity derived from hTERT and RMRP in vitro, Recombinant hTERT protein and RAMP transcribed in vitro were incubated under low salt or high salt conditions. The resulting products were treated with proteinase K followed by purification with phenol/chloroform treatment and then resolved by electrophoresis on a 7M Urea 5% polyacrylamide gel electrophoresis (PAGE).

FIG. 3E is a photograph depicting recombinant hTERT protein and RMRP transcribed in vitro were incubated under high salt conditions, treated with RNase T1, and resolved by 7M area 5% PAGE.

FIG. 3F is a photograph of gel electrophoresis in which the products of an RdRP assay performed in the presence of all four ribonucleotides (middle) or in the absence of adenine (left lane) or guanine (right lane) ribonucleotides, are separated by size. A and G are present within the first 5 nt of the predicted complementary strand of RMRP.

FIGS. 4A-D are photographs of gel electrophoresis separating RNA templates by size (A and C) and corresponding graphs (B and D) depicting the size calibration data based on the migration of the markers. To confirm that the predicted 2× template sized band migrates at the predicted size (534 nt), RNA products synthesized in vitro by the hTERT-RMRP RdRP together with several defined size markers were resolved by electrophoresis on a 7M Urea 5% polyacrylamide gel electrophoresis (PAGE). Panels (B) and (D) depict the calibration data (semi-logarithmic analysis) based on the migration of markers in PAGE from panels (A) and (C), respectively. Red lines (panels B and D) indicate the migration of the 2× size band at a position that corresponds to 534 nt. To ensure that the gel migrated in a straight line, the 380 nt markers (in panel A), 267 nt markers (in panel B) and 120 nt markers (in panel B) were applied in duplicate on opposite sides of the gel.

FIG. 5A is a photograph of gel electrophoresis depicting the products of RdRP activity separated by size, hTERT and RMRP are required for the RdRP activity. Reactions were performed under high salt conditions. No RdRP activity was detected in samples containing hTERC or the recombinant hTERT truncation mutant (GST-HT1).

FIG. 5B is a photograph of gel electrophoresis depicting the components of hTERT-RMRP complexes and products of RdRP activity separated by size. FLAG-tagged hTERT or FLAG-tagged dominant negative (DN) hTERT proteins were transiently expressed in 293T cells and immunoprecipitated using anti-FLAG-M2 antibody conjugated agarose beads. Immune complexes were either subjected to SDS-PAGE followed by the detection by immunoblotting with the FLAG-M2 antibody (upper panel) or associated RNAs were recovered and then subjected to RT-PCR. Recombinant hTERT (wild type or DN) protein and RMRP that had been transcribed in vitro were incubated under high salt conditions and the resulting products were treated with proteinase K followed by purification with phenol/chloroform treatment and then resolved by electrophoresis on a 7M Urea 5% PAGE.

FIG. 5C is a photograph of northern blotting analysis used to detect complementary sequence of RMRP produced by RdRP activity. An RdRP assay was performed in vitro without radioactivity and resulting products were resolved by 7M urea 5% PAGE. RNA products were blotted with an isotope labeled RMRP sense strand probe. Intermediate length products, representing incompletely elongated products, are also detected by the probe used for the Northern blotting.

FIG. 5D is a photograph of a gel electrophoresis depicting the products of RdRP over time, separated by size. Time course of RdRP activity demonstrates primer extension from the 1×RMRP size to the 2×RMRP size.

FIG. 5E is a schematic representation of the 3′ primer extension assay. Sense RMRP RNA is incubated with RT without primers followed by amplification step with the sense primer. Single stranded DNA is detected only when the 3′ end forms a fold-back conformation.

FIG. 5F is a schematic representation of RMRP truncation mutants and a photograph of gel electrophoresis in which products of a 3′ primer extension assay are separated by size. The of truncation mutants of RMRP (upper panel) were transcribed in vitro by SP6 polymerase then subjected to 3′ primer extension assay. Each RNA transcribed in vitro was used as a template for the 3′ extension assay (indicated on the lower panel) and resulting single stranded DNA species were resolved by denaturing PAGE (lower panel).

FIG. 5G is a photograph of gel electrophoresis in which RNA products produced by the RdRP activity derived from hTERT and total RNAs in vitro, are separated by size. Recombinant hTERT (wild type or DN) protein and total RNAs from either HeLa cells or 293T cells were incubated with ³²P-UTP and resulting products were treated with proteinase K, purified by phenol/chloroform treatment and resolved by electrophoresis on a 7M Urea 5% PAGE. Only a limited pool of RNAs serves a templates for RdRP activity.

FIG. 6A is a photograph of northern blotting analysis used to detect complementary sequence of RMRP in cell lines. RNA isolated from 293T cells, HeLa cells and MCF7 cells were treated with DNase I, resolved by 7M urea 5% PAGE. RNA products were blotted with a ³²-P labeled RMRP sense strand probe. Samples, indicated with a (+), were treated with RNase to ensure that the detected products were RNA.

FIG. 6B is a photograph of northern blotting analysis used to detect sense strand sequence of RMRP in cell lines. RNA isolated from 293T cells, HeLa cells and MCF7 cells were treated with DNase I, resolved by 7M urea 5% PAGE. RNA products were blotted with a ³²P-labeled RMRP antisense strand probe. Samples that were treated with RNase are indicated with a (+).

FIG. 6C is a photograph of gel electrophoresis in which the products of ectopic hTERT expression are separated by size. hTERT expression correlates with the levels of antisense RMRP detected by RNase protection assay (RPA). VA-13 control and BJ control indicated cells infected with control vectors and selected by exposure to hygromycin. hTERT levels were measured by RT-PCR.

FIG. 6D is a photograph of northern blotting analysis. hTERT expression correlates with the levels of 2× template sized products detected by Northern blotting. The relative signal intensity of the 2× template sized products is indicated below the panel.

FIG. 7 is a northern blotting analysis to detect sense strand sequence of RMRP produced by RdRP activity. An RdRP assay was performed in vitro, and the resulting products were resolved by 7M urea 5% PAGE. RNA products were blotted with an isotope labeled RMRP antisense strand probe. The background of this experiment is due to the presence of 1× templated sized sense strand RMRP and intermediate length products detected by this probe. An arrow indicates the 2× size band.

FIG. 8A is a photograph of northern blotting analysis. To confirm that the 2× template sized band migrates at the predicted size (534 nt), RNAs extracted from 293T cells and HeLa cells were subjected to electrophoresis on 7M Urea 5% polyacrylamide gel electrophoresis (PAGE) and then performed Northern blotting with a RMRP sense strand-specific probe.

FIG. 8B is a graph of the calibration data (semi-logarithmic analysis) based on the migration of molecular weight standards in FIG. 5A. Red line indicates that the predicted 2× size band corresponds to the correct position on the calibration.

FIG. 9 is a photograph of an RNAse protection assay. Controls to ensure the sensitivity and specificity of the RNase protection assay for RMRP (FIG. 6C). A negative control; luciferase probe (specific for a sequence not expected to be expressed in the cell lines) (left panel) and a positive control; β-actin probe (specific for a sequence known to be expressed in the cell lines) (right panel) are shown.

FIG. 10A is an agaraose gel image of the products of RT-PCR for total RMRP (upper panel) and retrovirally delivered RMRP (ectopic, lower panel) cell lines expressing control or RMRP expression vectors. Total RMRP was detected using primers that amplify both endogenous and ectopically introduced RMRP, ectopically expressed RMRP was detected with vector specific primers. Ectopically introduced RMRP was placed under the control of the promoters indicated on the panel. The relative signal intensity of total RMRP (control:RMRP) is 1:1.6 (VA-13), 1:0.4 (BJ) and 1:0.7 (HeLa), respectively.

FIG. 10B is an agaraose gel image of the products of RT-PCR for total RMRP from cell lines expressing control, hTERT (VA-13 cells) or expressing control sh-RNA, hTER T-specific shRNAs (HeLa cells), The relative signal intensity of RMRP is 1:0.3 (control:hTERT, VA-13) and 1:1.8:1.9 (sh-GFP:sh-hTERT#1:sh-hTERT#2, HeLa), respectively.

FIG. 10C is a photograph of gel electrophoresis analysis depicting levels of RMRP and protein expression. Effects of expressing truncated RMRP mutants on endogenous RMRP levels, RMRP mutants were introduced by retroviral infection and were driven by the LTR promoter. The relative signal intensity of RMRP is 1:0.5 (control:RMRP 1-267), 1:0.6 (control:RMRP 110-267), 1:1.5 (control:RMRP 1-200) and 1:1.7 (control:RMRP 1-120), respectively.

FIG. 10 is a photograph of northern blotting analysis. Detection of small RNA species derived from full length RMRP. Northern blotting was performed to detect 2× template sized RNAs (upper panel) and small RNAs (14 nt-30 nt in length) using the antisense strand of RMRP as a probe (lower panel). Asterisks indicate specific signals corresponding to 19-26 nt in length. U6 RNA probes were used to assess sample loading in each lane. RNAs were resolved by electrophoresis on a 7M Urea 20% PAGE.

FIG. 10E is a photograph of northern blotting analysis. Effect of suppressing Dicer on small RNA species derived from full length RMRP Northern blotting was performed to detect small RNAs using the antisense strand of RMRP as a probe. Asterisk and arrowheads indicate specific signals corresponding to 19-26 nt in length. U6 RNA probes were used to assess sample loading in each lane. RNAs were resolved by electrophoresis on a 7M Urea 20% PAGE.

FIG. 10F is a photograph of gel electrophoresis showing RMRP and protein expression levels. RT-PCR for total RMRP from cell lines expressing control shRNA or Dicer-specific shRNAs. The relative signal intensity of RMRP is 1:3.7:2.9 (sh-GFP:sh-Dicer#1:sh-Dicer#2, 293T), 1:2.7:2.2 (sh-GFP:sh-Dicer#1:sh-DicerA2, HeLa) and 1:1.5 (sh-GFP:sh-Dicer#2, MCF7), respectively.

FIG. 10G is an agarose gel image of small RNA species derived from full length RMRP that were cloned and sequenced. Chemically synthesized siRNAs (double stranded RNAs) were created based on the identified sequences. Synthesized siRNAs were introduced by transfection, total RNA was extracted and RT-PCR with primers specific for RMRP was performed. The relative signal intensity of RMRP 1:0.4:0.2 (control:siRNA#1:siRNA#2, 293T), 1:07:0.3 (control:siRNA#1:siRNA#2, HeLa), and 1:0.4:0.3 (control:siRNA#1:siRNA#2, MCF7), respectively.

FIG. 11A is a series of agarose gel images showing the effects of hTERT-specific shRNAs on hTERT expression, RMRP-specific shRNAs on RMRP expression or hTERC-specific shRNAs on hTERC expression. HeLa cells were infected with a GFP-specific shRNA (sh-GFP), hTERT coding sequence-specific shRNAs (sh-hTERT #1 or #2), RMRP coding sequence-specific shRNAs (sh-RMRP #1 or #2) or hTERC coding sequence-specific shRNAs (sh-hTERC #1 or #2). After drug selection, total RNAs were extracted and RT-PCR was performed for the indicated genes.

FIG. 11B is a series of agarose gel images showing the effect of suppressing hTERT, RMRP or hTERC on the transcription of human α-satellites (alphoid) at centromeres. RNAs from cells expressing a control shRNA (sh-GFP), 2 independent hTERT-specific shRNAs, 2 independent RMRP-specific shRNAs or 2 independent hTERC-specific shRNAs were isolated and transcripts from the alphoid loci were measured by RT-PCR.

FIG. 11C is a series of immunofluorescent photographs showing the effects of hTERT or RAMP suppression on trimethylation of histone H3 lysine 9 (H3-K9). Cells expressing a control shRNA (sh-GFP), 2 independent hTERT-specific shRNAs or 2 independent RMRP-specific shRNAs were stained with anti-trimethyl H3-K9 antibody. Green represents trimethylated H3-K9 staining and red represents DAPI staining. Asterisk indicates statistically significant differences.

FIG. 11D is a series of immunofluorescent photographs showing the effects of hTERT or RMRP suppression on HP1-β expression. Cells expressing a control shRNA (sh-GFP), 2 independent hTERT-specific shRNAs or 2 independent RAMP-specific shRNAs were stained with an anti-HP1-β antibody. Green represents HP1-β staining, and blue represents DAPI staining. Asterisk indicates statistically significant differences. The inset picture shows a higher magnification view.

FIG. 11E is a series of immunofluorescent photographs showing the effects of hTERT or RMRP suppression on acetylation of histone H3 lysine 9/14 (H3-K9/14 acetyl). Cells expressing a control shRNA (sh-GFP), an hTERT-specific shRNA or an RMRP-specific shRNA were stained with an antibody that recognizes acetylation of histone H3 on K9 and K14 lysines. Green represents H3-K9/14 acetylation, and blue represents DAPI staining. Numbers indicated under each panel represent relative fluorescent intensity (Mean±S.D.). The inset picture shows a higher magnification view.

FIG. 11F is a series of immunofluorescent photographs showing the effects of hTERT or RMRP suppression on CENP-A. Cells expressing a control shRNA (sh-GFP), an hTERT-specific shRNA or an RMRP-specific shRNA were stained with an anti-CENP-A antibody. Green represents CENP-A staining and blue represents DAPI staining. Numbers indicated under each panel represent relative fluorescent intensity (Mean±S.D.). The inset picture shows a higher magnification view.

FIG. 11G is a photograph showing the effects of hTERT or RMRP suppression on CENP-A were measured by immunoblotting. The relative signal intensity CENP-A is indicated below the gel. The inset pictures in (D), (E), and (F) show a higher magnification view of each panel.

FIG. 12 is an agarose gel image in which the products of the micrococcal nuclease (MN) digestion of nuclei derived from cells expressing the indicated shRNA vectors are separated by size. Nuclei isolated from 1×10⁶ cells were treated with MN for the indicated time, subjected to gel electrophoresis and stained with ethidium bromide. Arrowhead indicates the migration of mononucleosomes. It is noted that a faint signal is seen starting at 1 min in cells expressing sh-hTERT#1 or sh-RMRP#1, while comparable signals are observed at 3 min in control cells (indicated by asterisks). Moreover, MNase digests total chromatin into mononucleosomes more efficiently in cells expressing sh-hTERT#1 or sh-RMRP#1 than in cells expressing a control shRNA (sh-GFP) at 15 (circles) and 30 (arrows) min.

FIG. 13 is an immunofluorescent image showing the effects of hTERC suppression on trimethylation of histone H3 lysine 9 (H3-K9-trimethyl). Green represents trimethyl H3-K9 staining and blue represents DAPI staining. Numbers indicated under each panel represent relative fluorescent intensity (Mean±S.D.)

FIG. 14. Purification of GST-WT-hTERT and GST-DN-hTERT.

A, To optimize conditions to express GST-hTERT in E. coli, we tested the timing and effects of IPTG induction on expression levels. Exponentially growing cultures (See Methods) were incubated for the indicated time in the presence or absence of IPTG. Maximum expression was observed at 4 hr without IPTG induction.
B, Under the experimental conditions used above, we confirmed that soluble GST-WT-hTERT and GST-DN-hTERT were expressed at the same levels. Unbound: Supernatant after incubation with GST-Sepharose confirms that the majority of GST-WT- or DN-hTERT was bound to GST-Sepharose. Resin bound: An aliquot of the GST-Sepharose after incubation with the bacterial lysate shows that similar amounts of GST-WT- and DN-hTERT were bound. Elution 1-4: After binding GST-WT- or GST-DN-hTERT, the GST-Sepharose was eluted with 20 mM glutathione (reduced form) four times in elution buffer [50 mM Tris-HCl pH8.8, 150 mM NaCl, 0.5% NP-40, 0.1 mM DTT, 10 mM PMSF, proteinase inhibitor (nacalai tesque)]. Final resin: An aliquot of the GST-Sepharose after elution was denatured by incubation at 95° C. for 5 min, Nearly all of the GST hTERT was eluted under these conditions. For all gels, 8% SDS-PAGE was performed, and WT-hTERT or DN-hTERT was detected by immunoblotting with an anti-hTERT antibody (Rockland).

FIG. 15 Effects of double stranded RNA produced by the hTERT-RMRP RdRP and identification of small RNAs as siRNA.

A, Semi-quantitative RT-PCR for total RMRP (upper panel) and retrovirally delivered RMRP (ectopic, lower panel) in cell lines expressing control or RMRP expression vectors. Total RMRP was detected using primers that amplify both endogenous and ectopically introduced RMRP, ectopically expressed RMRP was detected with vector specific primers. Ectopically introduced RMRP was placed under the control of the promoters indicated on the panel. The relative signal intensity of total RMRP (control:RMRP) is 1:1.6 (VA-13), 1:0.4 (BJ), 1:0.7 (HeLa) and 1:0.7 (MCF7), respectively.
B, Quantitative RT-PCR using primers specific for total RMRP performed in cell lines expressing control or RMRP expression vectors. Ectopically introduced RMRP was placed under the control of the promoters indicated on the panel. Values represent mean±SD for three independent experiments. Northern blotting was also performed and the relative signal intensity assessed by Northern blotting is indicated below the gel. p values for the differences were calculated using Student's t-test. These Northern blotting and qRT-PCR experiments confirmed the differences in RMRP levels that were observed using the RT-PCR conditions used in FIG. 15A accurately reflect RMRP levels.
C, RT-PCR (left) and quantitative RT-PCR (right) for total RMRP from cell lines expressing a control vector or hTERT. The relative signal intensity of RMRP measured by RT-PCR was 1:0.3 (control:hTERT, VA-13) and 1:0.6 (control:hTERT, BJ).

FIG. 16 Effects of double stranded RNA produced by the hTERT-RMRP RdRP and identification of small RNAs as siRNA.

A, Detection of small RNA species in human cells. Northern blotting was performed to detect small RNAs (22 nt in length) using antisense (left panel) and sense (rightpanel) probes derived from nt 21-40 of RMRP. We note that the levels of the sense and antisense strands are different in these cell lines.
B and C, Analysis of the termini of the short RNA species identified in (A). Total RNA was isolated from the indicated cells and then incubated with the indicated enzyme (B) or oxidation-β-elimination reactions (C) were performed, and resolved by electrophoresis on 7M Urea 20% PAGE. Small RNAs were detected by Northern blotting with antisense probe. CIP=calf intestinal phosphatase. PNK=polynucleotide kinase. ATP— indicates samples where ATP was not added.

FIG. 17. Calibration of Northern blotting probes for hTERC and RMRP. hTERC RNA or RMRP RNA transcribed in vitro and the indicated amount of RNAs were resolved in 7M Urea 5% PAGE, and Northern blotting was performed with hTERC or RMRP probes (left panel). To compare the relative abundance of these RNAs in cells, total RNAs from each cell line were resolved by 7M Urea 5% PAGE, and Northern blotting with hTERC or RMRP probes was performed. We concluded that hTERC levels are five- to ten-fold higher than RMRP in these cells (right panel).

FIG. 18. Confirming the specificity of the probes used for strand specific Northern blotting.

A, To confirm the specificity of the probes used for Northern blotting, hTERC RNA (a negativecontrol), sense strand-RMRP RNA or antisense strand-RMRP RNA transcribed in vitro by SP6 polymerase were resolved by 7M Urea 5% PAGE, and Northern blotting was performed with the probes indicated.
B, To confirm the specificity of the probes used in Northern blotting for siRNA, synthesized RNA corresponding to the sense strand-RMRP RNA (20-41 nt) or to the antisense strand-RMRP RNA (20-41 nt) or an irrelevant RNA (synthesized 22 nt RNA:5′-gcuacauguggcuaacaugucg-3′) were resolved by electrophoresis on a 7M Urea 20% PAGE, and Northern blotting was performed with the probes indicated.

FIG. 19. Calibration of the sense+antisense RMRP products in RNA sextracted from cell lines.

A, To confirm that the sense+antisense band migrates at the predicted size (534 nt), we subjected RNAs extracted from 293T cells and HeLa cells to electrophoresis on 7M Urea 5% polyacrylamide gel electrophoresis (PAGE) and then performed Northern blotting with a RMRP sense strand probe.
B, The calibration data (semi-logarithmic analysis) based on the migration of molecular weight standards. Red line indicates that the predicted sense+antisense RMRP band corresponds to the correct position on the calibration.

FIG. 20. Control experiments for RNase protection assay.

Calibration of the RNase protection assay for antisense RMRP. The antisense strand of RMRP was transcribed in vitro (SP6), and the indicated amount of the RNA was hybridized overnight at 60° C. with ³²P-labeled RMRP sense probe. Hybrids were digested with RNase A and RNase T1. The protected fragments were separated by PAGE under denaturing conditions and visualized by autoradiography.

FIG. 21

hTERT expression correlates with the levels of the sense+antisense RMRP products detected by Northern blotting in 2 different cell lines. The bottom panel shows U2 RNA levels to ensure equal loading. The membrane for the sense probe was stripped and re-probed with the antisense probe.

FIG. 22, Calibration of the sense+antisense RMRP products produced invitro RdRP assay. To confirm that the sense+antisense RMRP band migrates at the predicted size (534 nt); RNA products synthesized in vitro by the hTERT-RMRP RdRP together with the indicated sizemarkers were resolved by electrophoresis on formaldehyde agarose gel. Panel (B) depict the calibration data (semi-logarithmic analysis) based on the migration of markers from panel (A). Red line (panel B) indicates the migration of the sense+antisense RMRP band at a position that corresponds to ˜534 nt.

FIG. 23

Recombinant hTERT protein and RMRP transcribed in vitro were incubated with ³²P-UTP and unlabeled ribonucleotides for the RdRP assay, the resulting products were treated with bacterial RNase III and resolved by 7M urea 5% PAGE. We note that the 10-11 nt fragments produced by RNase III are not shown.

FIG. 24. Time dependent extension of labeled RMRP.

³²P-labeled sense RMRP, recombinant hTERT protein and unlabeled ribonucleotides were incubated, and an RdRP assay was performed in vitro. The RdRP assay assayed at indicated timepoints and the products separated on 7M urea 5% PAGE.

FIG. 25 Production of RMRP-derived endogenous siRNAs depends on Dicer and RISC.

Effect of suppressing Dicer on RMRP-derived small RNAs. Northern blotting was performed to detect [1] small RNAs using the antisense strand of RMRP as a probe in HeLa, 2931 or MCF7 cells expressing control shRNA (sh-GFP) or Dicer-specific shRNAs (sh-Dicer #1 and sh-Dicer #2), [2] pre-miR-16 and mature miR-16 using a miR-16 specific probe, and [3] U6 RNA. The relative signal intensity of these small RNAs was 1:0.1:0.09 (sh-GFP:sh-Dicer#1:sh-Dicer #2,HeLa), 1:0.4:0.4 (sh-GFP:sh-Dicer#1:sh-Dicer#2, 2931), 1:0.5:0.4 (sh-GFP:sh-Dicer#2:sh-Dicer#2, MCF7), respectively. We note that suppression of Dicer induced a decrease in the levels of mature miR-16 similar to that observed in the RMRP-specific siRNAs and an increase levels of pre-miR-16. The relative signal intensity of the miR-16 is 1:0.2:0.2 (sh-GFP:sh-Dicer#1:sh-Dicer#2, HeLa), 1:0.4:0.2 (sh-GFP:sh-Dicer#1:sh-Dicer#2,293T), and 1:0.5:0.2 (sh-GFP:sh-Dicer#1:sh-Dicer#2, MCF7), respectively. U6 RNA was used to assess sample loading in each lane. RNAs were resolved by electrophoresis on a 7M Urea 20% PAGE.

FIG. 26. Production of RMRP-derived endogenous siRNAs depends on Dicer and RISC.

A, RT-PCR for total RMRP from cell lines expressing control shRNA or Dicer-specific shRNAs. The relative signal intensity of RMRP is 1:2.7:2.2 (sh-GFP:sh-Dicer#1:sh-Dicer#2, HeLa), 1:3.7:2.9 (sh-GFP:sh-Dicer#1:sh-Dicer#2, 293T), 1:1.5 (sh-GFP:sh-Dicer#2, MCF7), and 1:1.0:1.1 (sh-GFP:sh-Dicer#1:sh-Dicer#2, VA-13), respectively.
B, Re-introduction of chemically synthesized siRNA (double stranded RNAs) targeting 20-40 nt portion of the RMRP sequence suppresses RMRP. Using ten consecutive probes corresponding to the RMRP sequence, the small RNAs derived from RMRP were detected by probes containing the complementary sequences to nucleotides 21-40 of RMRP. A siRNA corresponding to this sequence was synthesized and introduced by transfection into the indicated cells; total RNA was extracted; and quantitative RT-PCR, using primers specific for total RMRP was performed. p values for the differences were calculated using Student's t-test.
C, RMRP-derived small RNAs are associated with hAgo2 in human cells, hAgo2 immune complexes were isolated from HeLa or 293T cells using anti-hAgo2-specific antisera or pre-immune sera RNA was isolated from these immune complexes and resolved by on 7M Urea 20% PAGE, Small RNAs were detected by Northern blotting with the indicated probes to detect: RMRP sense strand, top panel; RMRP anti-sense strand, middle panel; and mature miR-16, bottom panel. Synthesized oligonucleotides (RMRP 20-41 and RMRP AS 41-20) corresponding to the each probe were resolved by electrophoresis (also see FIG. 18B) were used to confirm the specificity of each probe. The migration of the 22 nt molecular mass marker is shown.

FIG. 27. Effects of suppressing Dicer on the levels of small RNAs.

As described in FIG. 25, control (sh-GFP) or Dicer-specific (sh-Dicer #1 and sh-Dicer #2) shRNAs were stably introduced into the indicated cells, and total RNA was isolated. The relative signal intensity of the small RNA species detected by a probe specific for RMRP (black bars) or by a probe for miR-16 (white bars) as assessed by Northern blotting as shown in FIG. 25. Signal intensity was determined for each probe by densitometry and normalized to the signal found for sh-GFP in each cell line.

FIG. 28. Effect of suppressing Dicer on sense+antisense RAMP RNAs.

Northern blotting was performed to detect the ˜534 nt sense±antisense RMRP RNAs with a ³²P-labeled RMRP sense strand probe. RNAs in HeLa, 293T or MCF7 cells expressing control shRNA (sh-GFP) or Dicer-specific shRNAs (sh-Dicer #1 and sh-Dicer #2) were isolated and resolved by 7M urea 5% PAGE.

### MODE FOR CARRYING OUT THE INVENTION

Constitutive expression of telomerase in human cells prevents the onset of senescence and crisis by maintaining telomere homeostasis. Moreover, the human telomerase catalytic subunit (hTERT) contributes to cell physiology independent of its ability to elongate telomeres. The invention is based upon the unexpected discovery that hTERT interacts with the RNA component of mitochondrial RNA processing endoribonuclease (RMRP), a gene that is mutated in the inherited pleiotropic syndrome Cartilage-Hair Hypoplasia. Furthermore, hTERT and RMRP form an RNA dependent RNA polymerase (RdRP) and produce double-stranded RNAs that can be processed into small interfering RNA. Expression of the RdRP formed by hTERT and RMRP is necessary to silence human centromeric satellite repeat regions and participates in maintaining heterochromatin. These results identify a mammalian RdRP composed of hTERT in complex with RMRP that participates in the regulation of chromatin structure. This is the first mammalian RdRP described.

Telomerase is a ribonucleoprotein complex that elongates telomeres and protects chromosome ends. Although several proteins interact with telomerase, the minimal components of telomerase required for the synthesis of telomeric repeats include the catalytic telomerase reverse transcriptase (TERT) and a non-coding telomerase RNA subunit (telomerase RNA component; TERC) that encodes the template for the synthesis of telomeric DNA. Telomere homeostasis mediated by telomerase serves to maintain genomic stability and regulates human cell lifespan. Indeed, mutations in hTERT, hTERC or dyskerin, a nucleolar protein associated with telomerase and involved in rRNA maturation, are found in the various forms of dyskeratosis congenita, a syndrome characterized by ectodermal dysplasia and bone marrow failure (Calado, R. T. and Young, N. S. Blood 111) 4446 (2008)). Moreover, alterations in the regulation of telomeres and telomerase contribute to malignant transformation by affecting both genomic integrity and cell immortalization (Chan, S. W. and Blackburn, E. H. Oncogene 21, 553 (2002); Shay, J. W. and Wright, W E. J Pathol 211, 114 (2007)).

hTERT exhibits other activities beyond its role in telomere homeostasis and forms several intracellular complexes (Fu, D. and Collins, K. Mol Cell 28, 773 (2007); Venteicher, A. S. et al. Cell 132, 945 (2008)). Overexpression of hTERT induces increased tumor susceptibility (Gonzalez-Suarez, E. et al., EMBO J. 20, 2619 (2001); Artandi, S. E, et al., Proc Natl Acad Sci U S A 99, 8191 (2002)) and disrupts normal stem cell function independent of telomere maintenance (Sarin, K. Y. et al., Nature 436, 1048 (2005); Blackburn, E. H. Nature 436. 922 (2005)) while suppression of hTERT expression or inhibiting hTERT activity alters global chromatin structure (Masutomi, K. et al., Proc Natl Acad Sci USA 102, 8222 (2005)).

Accordingly, the invention provides compositions and methods of increasing or decreasing gene silencing in a cell as well as methods of treating diseases which are either caused by the inappropriate deactivation/silencing of a gene or the by the undesired or overexpression of a gene.

hTERT

Compositions and methods of the invention include a TERT subunit or fragments thereof. The TERT subunit is, for example, human TERT (hTERT). Exemplary hTERT subunits encompassed by the invention include, but are not limited to, those polypeptides encoded by the mRNA and amino acid sequences below (SEQ ID NOs:1-4). One exemplary fragment of hTERT that is used in the compositions and methods of the invention is the amino terminal end (amino acids 1-531) of either SEQ ID NO: 2 or 4, that is required for hTERT to interact with RMRP. Two additional fragments of hTERT that are included or removed in the compositions and methods of the invention are within the amino terminal end (amino acids 30-159 and 350-547) of either SEQ ID NO: 2 or 4, both of which are required for hTERT to interact with hTERC.

Human TERT, transcript variant 1, is encoded by the following mRNA sequence (NCBI Accession No. NM_{—}198253 and SEQ ID NO: 1)(all sequences provided herein are given from 5′ to 3′):

| | |
|---|---|
| 1 | caggcagcgc tgcgtcctgc tgcgcacgtg ggaagccctg gccccggcca cccccgcgat |
| 61 | gccgcgcgct ccccgctgcc gagccgtgcg ctccctgctg cgcagccact accgcgaggt |
| 121 | gctgccgctg gccacgttcg tgcggcgcct ggggccccag ggctggcggc tggtgcagcg |
| 181 | cggggacccg gcggctttcc gcgcgctggt ggcccagtgc ctggtgtgcg tgccctggga |
| 241 | cgcacggccg ccccccgccg ccccctcctt ccgccaggtg tcctgcctga aggagctggt |
| 301 | ggcccgagtg ctgcagaggc tgtgcgagcg cggcgcgaag aacgcgctgg ccttcggctt |
| 361 | cgcgctgctg gacggggccc gcgggggccc ccccgaggcc ttcaccacca gcgtgcgcag |
| 421 | ctacctgccc aacacggtga ccgacgcact gcgggggagc ggggcgtggg ggctgctgct |
| 481 | gcgccgcgtg ggcgacgacg tgctggttca cctgctggca cgctgcgcgc tctttgtgct |
| 541 | ggtggctccc agctgcgcct accaggtgtg cgggccgccg ctgtaccagc tcggcgctgc |
| 601 | cactcaggcc cggcccccgc cacacgctag tggaccccga aggcgtctgg gatgcgaacg |
| 661 | ggcctggaac catagcgtca gggaggccgg ggtccccctg ggcctgccag ccccgggtgc |
| 721 | gaggaggcgc gggggcagtg ccagccgaag tctgccgttg cccaagaggc ccaggcgtgg |
| 781 | cgctgcccct gagccggagc ggacgcccgt tgggcagggg tcctgggccc acccgggcag |
| 841 | gacgcgtgga ccgagtgacc gtggtttctg tgtggtgtca cctgccagac ccgccgaaga |
| 901 | agccacctct ttggagggtg cgctctctgg cacgcgccac tcccacccat ccgtgggccg |
| 961 | ccagcaccac gcgggccccc catccacatc gcggccacca cgtccctggg acacgccttg |
| 1021 | tcccccggtg tacgccgaga ccaagcactt cctctactcc tcaggcgaca aggagcagct |
| 1081 | gcggccctcc ctcctactca gctctctgag gcccagcctg actggcgctc ggaggctcgt |
| 1141 | ggagaccatc tttctgggtt ccaggccctg gatgccaggg actccccgca ggttgccccg |
| 1201 | cctgccccag cgctactggc aaatgcggcc cctgtttctg gagctgcttg ggaaccacgc |
| 1261 | gcagtgcccc tacggggtgc tcctcaagac gcactgcccg ctgcgagctg cggtcacccc |
| 1321 | agcagccggt gtctgtgccc gggagaagcc ccagggctct gtggcggccc ccgaggagga |
| 1381 | ggacacagac ccccgtcgcc tggcgcagct gctccgccag cacagcagcc cctggcaggt |
| 1441 | gtacggcttc gtgcgggcct gcctgcgccg gctggtgccc ccaggcctct ggggctccag |
| 1501 | gcacaacgaa cgccgcttcc tcaggaacac caagaagctc atctccctgg ggaagcacgc |
| 1561 | caagctctcg ctgcaggagc tgacgtggaa gatgagcgtg cgggactgcg cttggctgcg |
| 1621 | caggagccca ggggttggct gtgttccggc cgcagagcac cgtctgcgtg aggagatcct |
| 1681 | ggccaagttc ctgcactggc tgatgagtgt gtacgtcgtc gagctgctca ggtctttctt |
| 1741 | ttatgtcacg gagaccacgt ttcaaaagaa caggctcttt ttctaccgga agagtgtctg |
| 1801 | gagcaagttg caaagcattg gaatcagaca gcacttgaag agggtgcagc tgcgggagct |
| 1861 | gtcggaagca gaggtcaggc agcatcggga agccaggccc gccctgctga cgtccagact |
| 1921 | ccgcttcatc cccaagcctg acgggctgcg accgattgtg aacatggact acgtcgtggg |
| 1981 | agccagaacg ttccgcagag aaaagagggc cgagcgtctc acctcgaggg tgaaggcact |
| 2041 | gttcagcgtg ctcaactacg agcgggcgcg gcgccccggc ctcctgggcg cctctgtgct |
| 2101 | gggcctggac gatatccaca gggcctggcg caccttcgtg ctgcgtgtgc gggcccagga |
| 2161 | cccgccgcct gagctgtact ttgtcaaggt ggatgtgacg ggcgcgtacg acaccatccc |
| 2221 | ccaggacagg ctcacggagg tcatcgccag catcatcaaa ccccagaaca cgtactgcgt |
| 2281 | gcgtcggtat accgtggtcc agaaggccgc ccatgggcac gtccgcaagg ccttcaagag |
| 2341 | ccacgtctct accttgacag acctccagcc gtacatgcga cagttcgtgg ctcacctgca |
| 2401 | ggagaccagc ccgctgaggg atgccgtcgt catcgagcag agctcctccc tgaatgaggc |
| 2461 | cagcagtggc ctcttcgacg tcttcctacg cttcatgtgc caccacgccg tgcgcatcag |
| 2521 | gggcaagtcc tacgtccagt gccaggggat cccgcagggc tccatcctct ccacgctgct |
| 2581 | ctgcagcctg tgctacggcg acatggagaa caagctgttt gcgggaattc ggcgggacgg |
| 2641 | gctgctcctg cgtttggtgg atgatttctt gttggtgaca cctcacctca cccacgcgaa |
| 2701 | aaccttcctc aggaccctgg tccgaggtgt ccctgagtat ggctgcgtgg tgaacttgcg |
| 2761 | aaagacagtg gtgaacttcc ctgtagaaga cgaggccctg ggtggcacgg cttttgttca |
| 2821 | gatgccggcc cacggcctat tcccctggtg cggcctgctg ctggataccc ggaccctgga |
| 2881 | ggtgcagagc gactactcca gctatgcccg gacctccatc agagccagtc tcaccttcaa |
| 2941 | ccgcggcttc aaggctggga ggaacatgcg tcgcaaactc tttggggtct tgcggctgaa |
| 3001 | gtgtcacagc ctgtttctgg atttgcaggt gaacagcctc cagacggtgt gcaccaacat |
| 3061 | ctacaagatc ctcctgctgc aggcgtacag gtttcacgca tatgtgctgc agctcccatt |
| 3121 | tcatcagcaa gtttggaaga accccacatt tttcctgcgc gtcatctctg acacggcctc |
| 3181 | cctctgctac tccatcctga aagccaagaa cgcagggatg tcgctggggg ccaagggcgc |
| 3241 | cgccggccct ctgccctccg aggccgtgca gtggctgtgc caccaagcat tcctgctcaa |
| 3301 | actgactcga caccgtgtca cctacgtgcc actcctgggg tcactcagga cagcccagac |
| 3361 | gcagctgagt cggaagctcc cggggacgac actgactgcc ctggaggccg cagccaaccc |
| 3421 | ggcactgccc tcagacttca agaccatcct ggactgatgg ccacccgccc acagccaggc |
| 3481 | cgagagcaga caccagcagc cctgtcacgc cgggctctac gtcccaggga gggaggggcg |
| 3541 | gcccacaccc aggcccgcac cgctgggagt ctgaggcctg agtgagtgtt tggccgaggc |
| 3601 | ctgcatgtcc ggctgaaggc tgagtgtccg gctgaggcct gagcgagtgt ccagccaagg |
| 3661 | gctgagtgtc cagcacacct gccgtcttca cttccccaca ggctggcgct cggctccacc |
| 3721 | ccagggccag cttttcctca ccaggagccc ggcttccact ccccacatag gaatagtcca |
| 3781 | tccccagatt cgccattgtt cacccctcgc cctgccctcc tttgccttcc acccccacca |
| 3841 | tccaggtgga gaccctgaga aggaccctgg gagctctggg aatttggagt gaccaaaggt |
| 3901 | gtgccctgta cacaggcgag gaccctgcac ctggatgggg gtccctgtgg gtcaaattgg |
| 3961 | ggggaggtgc tgtgggagta aaatactgaa tatatgagtt tttcagtttt gaaaaaaa |

Human TERT, transcript variant 1, is encoded by the following amino acid sequence (NCBI Accession No. NP_{—}937983.2 and SEQ NO: 2):

MPRAPRCRAVRSLLRSHYREVLPLATFVRRLGPQGWRLVQRGDPAAFRAL
VAQCLVCVPWDARPPPAAPSFRQVSCLKELVARVLQRLCERGAKNVLAFG
FALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCE
RAWNHSVREAGVPLGLPAPGARRRGGSASRSLPLPKRPRRGAAPEPERTP
VGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSL
RPSLTGARRLVETIFLGSRPWMPGTPRRLPRLPQRYWQMRPLFLELLGNH
AQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKH
AKLSLQELTWKMSVRDCAWLRRSPGVGCVPAAEHRLREEILAKFLHWLMS
VYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKR
AERLTSRVKALFSVLNYERARRPGLLGASVLGLDDIHRAWRTFVLRVRAQ
DPPPELYFVKVDVTGAYDTIPQDRLTEVIASIIKPQNTYCVRRYAVVQKA
AHGHVRKAFKSHVSTLTDLQPYMRQFVAHLQETSPLRDAVVIEQSSSLNE
ASSGLFDVFLRFMCHHAVRIRGKSYVQCQGIPQGSILSTLLCSLCYGDME
NKLFAGIRRDGLLLRLVDDFLLVTPHLTHAKTFLRTLVRGVPEYGCVVNL
RKTVVNFPVEDEALGGTAFVQMPAHGLFPWCGLLLDTRTLEVQSDYSSYA
RTSIRASLTFNRGFKAGRNMRRKLFGVLRLKCHSLFLDLQVNSLQTVCTN
IYKILLLQAYRFHACVLQLPFHQQVWKNPTFFLRVISDTASLCYSILKAK
NAGMSLGAKGAAGPLPSEAVQWLCHQAFLLKLTRHRVTYVPLLGSLRTAQ
TQLSRKLPGTTLTALEAAANPALPSKFKTILD

Human TERT, transcript variant 2, is encoded by the following mRNA sequence (NCBI Accession No. NM_{—}198255 and SEQ ID NO: 3) (Isoform 2 is a dominant-negative inhibitor of telomerase activity.):

| | |
|---|---|
| 1 | caggcagcgc tgcgtcctgc tgcgcacgtg ggaagccctg gccccggcca cccccgcgat |
| 61 | gccgcgcgct ccccgctgcc gagccgtgcg ctccctgctg cgcagccact accgcgaggt |
| 121 | gctgccgctg gccacgttcg tgcggcgcct ggggccccag ggctggcggc tggtgcagcg |
| 181 | cggggacccg gcggctttcc gcgcgctggt ggcccagtgc ctggtgtgcg tgccctggga |
| 241 | cgcacggccg ccccccgccg ccccctcctt ccgccaggtg tcctgcctga aggagctggt |
| 301 | ggcccgagtg ctgcagaggc tgtgcgagcg cggcgcgaag aacgtgctgg ccttcggctt |
| 361 | cgcgctgctg gacggggccc gcgggggccc ccccgaggcc ttcaccacca gcgtgcgcag |
| 421 | ctacctgccc aacacggtga ccgacgcact gcgggggagc ggggcgtggg ggctgctgct |
| 481 | gcgccgcgtg ggcgacgacg tgctggttca cctgctggca cgctgcgcgc tctttgtgct |
| 541 | ggtggctccc agctgcgcct accaggtgtg cgggccgccg ctgtaccagc tcggcgctgc |
| 601 | cactcaggcc cggcccccgc cacacgctag tggaccccga aggcgtctgg gatgcgaacg |
| 661 | ggcctggaac catagcgtca gggaggccgg ggtccccctg ggcctgccag ccccgggtgc |
| 721 | gaggaggcgc gggggcagtg ccagccgaag tctgccgttg cccaagaggc ccaggcgtgg |
| 781 | cgctgcccct gagccggagc ggacgcccgt tgggcagggg tcctgggccc acccgggcag |
| 841 | gacgcgtgga ccgagtgacc gtggtttctg tgtggtgtca cctgccagac ccgccgaaga |
| 901 | agccacctct ttggagggtg cgctctctgg cacgcgccac tcccacccat ccgtgggccg |
| 961 | ccagcaccac gcgggccccc catccacatc gcggccacca cgtccctggg acacgccttg |
| 1021 | tcccccggtg tacgccgaga ccaagcactt cccctactcc tcaggcgaca aggagcagct |
| 1081 | gcggccctcc ttcctactca gctctctgag gcccagcctg actggcgctc ggaggctcgt |
| 1141 | ggagaccatc tttctgggtt ccaggccctg gatgccaggg actccccgca ggttgccccg |
| 1201 | cctgccccag cgctactggc aaatgcggcc cccgtttctg gagctgcttg ggaaccacgc |
| 1261 | gcagtgcccc tacggggtgc tcctcaagac gcactgcccg ctgcgagctg cggtcacccc |
| 1321 | agcagccggt gtctgtgccc gggagaagcc ccagggctct gtggcggccc ccgaggagga |
| 1381 | ggacacagac ccccgtcgcc tggtgcagct gctccgccag cacagcagcc cctggcaggt |
| 1441 | gtacggcttc gtgcgggcct gcctgcgccg gctggtgccc ccaggcctct ggggctccag |
| 1501 | gcacaacgaa cgccgcttcc tcaggaacac caagaagttc atctccctgg ggaagcatgc |
| 1561 | caagctctcg ctgcaggagc tgacgtggaa gatgagcgtg cgggactgcg cttggctgcg |
| 1621 | caggagccca ggggttggct gtgttccggc cgcagagcac cgtctgcgtg aggagatcct |
| 1681 | ggccaagttc ctgcactggc tgatgagtgt gtacgtcgtc gagctgctca ggtctttctt |
| 1741 | ttatgtcacg gagaccacgt ttcaaaagaa caggctcttt ttctaccgga agagtgtctg |
| 1801 | gagcaagttg caaagcattg gaatcagaca gcacttgaag agggtgcagc tgcgggagct |
| 1861 | gtcggaagca gaggtcaggc agcatcggga agccaggccc gccctgctga cgtccagact |
| 1921 | ccgcttcatc cccaagcctg acgggctgcg gccgattgtg aacatggact acgtcgtggg |
| 1981 | agccagaacg ttccgcagag aaaagagggc cgagcgtctc acctcgaggg tgaaggcact |
| 2041 | gttcagcgtg ctcaactacg agcgggcgcg gcgccccggc ctcctgggcg cctctgtgct |
| 2101 | gggcctggac gatatccaca gggcctggcg caccttcgtg ctgcgtgtgc gggcccagga |
| 2161 | cccgccgcct gagctgtact ttgtcaagga caggctcacg gaggtcatcg ccagcatcat |
| 2221 | caaaccccag aacacgtact gcgtgcgtcg gtatgccgtg gtccagaagg ccgcccatgg |
| 2281 | gcacgtccgc aaggccttca agagccacgt ctctaccttg acagacctcc agccgtacat |
| 2341 | gcgacagttc gtggctcacc tgcaggagac cagcccgctg agggatgccg tcgtcatcga |
| 2401 | gcagagctcc tccctgaatg aggccagcag tggcctcttc gacgtcttcc tacgcttcat |
| 2461 | gtgccaccac gccgtgcgca tcaggggcaa gtcctacgtc cagtgccagg ggatcccgca |
| 2521 | gggctccatc ctctccacgc tgctctgcag cctgtgctac ggcgacatgg agaacaagct |
| 2581 | gtttgcgggg attcggcggg acgggctgct cccgcgtttg gtggatgatt tcttgttggt |
| 2641 | gacacctcac ctcacccacg cgaaaacctt cctcaggacc ctggtccgag gtgtccctga |
| 2701 | gtatggctgc gtggtgaact tgcggaagac agtggtgaac ttccctgtag aagacgaggc |
| 2761 | cctgggtggc acggcttttg ttcagatgcc ggcccacggc ctattcccct ggtgcggcct |
| 2821 | gctgctggat acccggaccc tggaggtgca gagcgactac tccagctatg cccggacctc |
| 2881 | catcagagcc agtctcacct tcaaccgcgg cttcaaggct gggaggaaca tgcgtcgcaa |
| 2941 | actctttggg gtcttgcggc tgaagtgtca cagcctgttt ctggatttgc aggtgaacag |
| 3001 | cctccagacg gtgtgcacca acatctacaa gatcctcctg ctgcaggcgt acaggtttca |
| 3061 | cgcatgtgtg ctgcagctcc catttcatca gcaagtttgg aagaacccca catttttcct |
| 3121 | gcgcgtcatc tctgacacgg cctccctctg ctactccatc ctgaaagcca agaacgcagg |
| 3181 | gatgtcgctg ggggccaagg gcgccgccgg ccctctgccc tccgaggccg tgcagtggct |
| 3241 | gtgccaccaa gcattcctgc tcaagctgac tcgacaccgt gtcacctacg tgccactcct |
| 3301 | ggggtcactc aggacagccc agacgcagct gagtcggaag ctcccgggga cgacgctgac |
| 3361 | tgccctggag gccgcagcca acccggcact gccctcagac ttcaagacca tcctggactg |
| 3421 | atggccaccc gcccacagcc aggccgagag cagacaccag cagccctgtc acgccgggct |
| 3481 | ctacgtccca gggagggagg ggcggcccac acccaggccc gcaccgctgg gagtctgagg |
| 3541 | cctgagtgag tgtttggccg aggcctgcat gtccggctga aggctgagtg tccggctgag |
| 3601 | gcctgagcga gtgtccagcc aagggctgag tgtccagcac acctgccgtc ttcacttccc |
| 3661 | cacaggctgg cgctcggctc caccccaggg ccagcttttc ctcaccagga gcccggcttc |
| 3721 | cactccccac ataggaatag tccatcccca gattcgccat tgttcacccc tcgccctgcc |
| 3781 | ctcctttgcc ctccaccccc accatccagg tggagaccct gagaaggacc ctgggagctc |
| 3841 | tgggaatttg gagtgaccaa aggtgtgccc tgtacacagg cgaggaccct gcacctggat |
| 3901 | gggggtccct gtgggtcaaa ttggggggag gtgctgtggg agtaaaatac tgaatatatg |
| 3961 | agtttttcag ttttgaaaaa aa |

Human TERT, transcript variant 2, is encoded by the following amino acid sequence (NCBI Accession No. NP_{—}937986.1 and SEQ ID NO: 4):

MPRAPRCRAVRSLLRSHYREVLPLATFVRRLGPQGWRLVQRGDPAAFRAL
VAQCLVCVPWDARPPPAAPSFRQVSCLKELVARVLQRLCERGAKNVLAFG
FALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCE
RAWNHSVREAGVPLGLPAPGARRRGGSASRSLPLPKRPRRGAAPEPERTP
VGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSL
RPSLTGARRLVETIFLGSRPWMPGTPRRLPRLPQRYWQMRPLFLELLGNH
AQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKH
AKLSLQELTWKMSVRDCAWLRRSPGVGCVPAAEHRLREEILAKFLHWLMS
VYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKR
AERLTSRVKALFSVLNYERARRPGLLGASVLGLDDIHRAWRTFVLRVRAQ
DPPPELYFVKDRLTEVIASIIKPQNTYCVRRYAVVQKAAHGHVRKAFKSH
VSTLTDLQPYMRQFVAHLQETSPLRDAVVIEQSSSLNEASSGLFDVFLRF
MCHHAVRIRGKSYVQCQGIPQGSILSTLLCSLCYGDMENKLFAGIRRDGL
LLRLVDDFLLVTPHLTHAKTFLRTLVRGVPEYGCVVNLRKTVVNFPVEDE
ALGGTAFVQMPAHGLFPWCGLLLDTRTLEVQSDYSSYARTSIRASLTFNR
GFKAGRNMRRKLFGVLRLKCHSLFLDLQVNSLQTVCTNIYKILLLQAYRF
HACVLQLPFHQQVWKNPTFFLRVISDTASLCYSILKAKNAGMSLGAKGAA
GPLPSEAVQWLCHQAFLLKLTRHRVTYVPLLGSLRTAQTQLSRKLPGTTL
TALEAAANPALPSDFKTILD

### RMRP

Compositions and methods of the invention include a RMRP or fragments thereof. Exemplary RMRPs encompassed by the invention include, but are not limited to, those polynucleotides encoded by the sequence below (SEQ ID NO: 5).

Human RNA component of mitochondrial RNA processing endoribonuclease (RMRP) is encoded by the following mRNA sequence (NCBI Accession No. NR_{—}003051 and SEQ ID NO: 5):

| | |
|---|---|
| 1 | gttcgtgctg aaggcctgta tcctaggcta cacactgagg actctgttcc tcccctttcc |
| 61 | gcctagggga aagtccccgg acctcgggca gagagtgcca cgtgcatacg cacgtagaca |
| 121 | ttccccgctt cccactccaa agtccgccaa gaagcgtatc ccgctgagcg gcgtggcgcg |
| 181 | ggggcgtcat ccgtcagctc cctctagtta cgcaggcagt gcgtgtccgc gcaccaacca |
| 241 | cacggggctc attctcagcg cggct |

### Compositions and TERT-RNA Complexes

The invention provides complexes containing a telomerase catalytic subunit (TERT) polypeptide, or fragment thereof and either a RNA component of the mitochondrial processing endoribonuclease (RMRP) or a mammalian RNA that forms a complex with TERT and has RNA-dependent RNA polymerase (RdRP) activity.

The TERT polypeptide is isolated from any source. In a preferred embodiment of the invention, the TERT polypeptide is human TERT (hTERT). However, all mammalian and eukaryotic TERT polypeptides are encompassed by the invention.

The RMRP and RNA elements of the compositions of the invention are isolated from any source. In a preferred embodiment of the invention, the RNA elements are human. The length of the RNA elements is not limited and is, for example, 1000 nucleotides or more, less than 1000 nucleotides, less than 500 nucleotides or less than 100 nucleotides.

As used herein, an “isolated” nucleic acid molecule, polynucleotide, polypeptide, protein, or complex can be substantially free of other cellular material, or culture medium when produced by recombinant techniques, or chemical precursors or other chemicals when chemically synthesized. An isolated polynucleotide is, for example, a recombinant RNA molecule, provided one of the nucleic acid sequences normally found immediately flanking that recombinant RNA molecule in a naturally-occurring molecule is removed or absent. Thus, isolated polynucleotides include, without limitation, a recombinant RNA that exists as a separate molecule (e.g., a cDNA or genomic DNA fragment produced by PCR or restriction endonuclease treatment) independent of other sequences as well as recombinant RNA that is incorporated into a vector, an autonomously replicating plasmid, a virus (e.g., a retrovirus, adenovirus, or herpes virus), or into the genomic RNA of a prokaryote or eukaryote, In addition, an isolated polynucleotide can include a recombinant RNA molecule that is part of a hybrid or fusion polynucleotide.

A nucleic acid molecule can be fused to other coding or regulatory sequences and still be considered “isolated”. Nucleic acid molecules present in nonhuman transgenic animals, which do not naturally occur in the animal, are also considered “isolated”. For example, recombinant nucleic acid molecules contained in a vector are considered “isolated”. Further examples of “isolated” nucleic acid molecules include recombinant DNA or RNA molecules maintained in heterologous host cells, and purified (partially or substantially) DNA or RNA molecules in solution. Isolated RNA molecules include in vivo or in vitro RNA transcripts of the isolated nucleic acid molecules of the present invention. Moreover, isolated RNA molecules include, but are not limited to, messenger RNA (mRNA), interfering RNA (RNAi), short interfering RNA (siRNA), short hairpain RNA (shRNA), double-stranded RNA (dsRNA), and microRNA (miRNA). Isolated nucleic acid molecules according to the present invention further include such molecules produced synthetically.

Isolated nucleic acid molecules, polypeptides, complexes, and compositions of the invention are associated with, bound to, conjugated to, linked to, or incorporated with a virus (or any part or fragment thereof), a liposome, a lipid, an antibody, an intrabody, a protein, a receptor, a ligand, a cytotoxic compound, a radioisotope, a toxin, a chemotherapeutic agent, a salt, an ester, a prodrug, a polymer, a hydrogel, a microcapsule, a nanocapsule, a microsphere, a cyclodextin, a plasmid, an expression vector, a proteinaceous vector, a detectable label (e.g. fluorescent, radioactive, magnetic, paramagnetic, etc.), an antigen, a diluent, an excipient, an adjuvant, an emulsifier, a buffer, a stabilizer, or a preservative.

As used herein, the term “fragment” is meant to describe an isolated nucleic acid or polypeptide molecule that is shorter in sequence the isolated nucleic acid or polypeptide molecule from which it is derived. Moreover, a fragment also describes a portion of a subunit or a complex that serves or has a particular function or characteristic, although the sequence comprised by that portion may not be continuous or contiguous, i.e. a polypeptide or polynucleotide binding surface.

Fragments of isolated nucleic acid and polypeptide molecules of the invention can contain, consist of, or comprise any part of the isolated nucleic acid or polypeptide molecule from which it is derived. A fragment typically comprises a contiguous nucleotide or polypeptide sequence at least about 8 or more nucleotides or amino acids, more preferably at least about 10 or more nucleotides or amino acids, and even more preferably at least about 16 or more nucleotides or amino acids. Further, a fragment could comprise at least about 18, 20, 21, 22, 25, 30, 40, 50, 60, 100, 250, 500, or 1000 (or any other number in-between) nucleotides or amino acids in length. The length of the fragment will be based on its intended use. A labeled probe can then be used, for example, to screen a cDNA library, genomic DNA library, or mRNA to isolate nucleic acid corresponding to the region or function of interest. Further, primers can be used in amplification reactions, such as for purposes of assaying one or more hTERT binding partners or for cloning specific regions of a gene.

An isolated nucleic acid molecule of the present invention further encompasses a polynucleotide that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874, 1990).

As used herein, an “amplified polynucleotide” of the invention is a isolated nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 10 nucleotides in length. More typically, an amplified polynucleotide is at least about 1.6 nucleotides in length. In a preferred embodiment of the invention, an amplified polynucleotide is at least about 2025 nucleotides in length. In a more preferred embodiment of the invention, an amplified polynucleotide is at least about 21, 22, 23, 24, 25, 30, 35, 40, 45, 50, or 60 nucleotides in length. In yet another preferred embodiment of the invention, an amplified polynucleotide is at least about 100, 200, or 300 nucleotides n length. While the total length of an amplified polynucleotide of the invention can be as long as an exon, an intron, a 5′ UTR, a 3′ UTR, or an entire gene, an amplified product is typically no greater than about 1,000 nucleotides in length (although certain amplification methods may generate amplified products greater than 1000 nucleotides in length). More preferably, an amplified polynucleotide is not greater than about 600 nucleotides in length.

Accordingly, the present invention provides nucleic acid molecules that consist of the nucleotide sequence of SEQ ID NOs: 1, 3, 5-35. A nucleic acid molecule consists of a nucleotide sequence when the nucleotide sequence is the complete nucleotide sequence of the nucleic acid molecule.

The present invention further provides polypeptide molecules that consist of the amino acid sequence of SEQ ID NOs: 2 and 4 as well as those polypeptide molecules encoded by the polynucleotide sequences of SEQ ID NOs: 1,3,5-35. A polypeptide molecule consists of an amino acid sequence when the amino acid sequence is the complete amino acid sequence of the polypeptide molecule.

The present invention further provides nucleic acid molecules that consist essentially of the nucleotide sequence of SEQ ID NOs: 1, 3, 5-35. A nucleic acid molecule consists essentially of a nucleotide sequence when such a nucleotide sequence is present with only a few additional nucleotide residues in the final nucleic acid molecule.

The present invention further provides polypeptide molecules that consist essentially of the amino acid sequence of SEQ ID NOs: 2 and 4 as well as those polypeptide molecules encoded by the polynucleotide sequences of SEQ ID NOs: 1, 3, 5-35. A polypeptide molecule consists essentially of an amino acid sequence when such amino acid sequence is present with only a few additional amino acid residues in the final nucleic acid molecule.

The present invention further provides nucleic acid molecules that comprise the nucleotide sequence of SEQ ID NOs: 3, 5-35. A nucleic acid molecule comprises a nucleotide sequence when the nucleotide sequence is at least part of the final nucleotide sequence of the nucleic acid molecule. In such a fashion, the nucleic acid molecule can be only the nucleotide sequence or have additional nucleotide residues, such as residues that are naturally associated with it or heterologous nucleotide sequences. Such a nucleic acid molecule can have one to a few additional nucleotides or can comprise many more additional nucleotides. A brief description of how various types of these nucleic acid molecules can be readily made and isolated is provided below, and such techniques are well known to those of ordinary skill in the art (Sambrook and Russell, 2000, Molecular Cloning: A Laboratory Manual, Cold Spring Harbor Press, NY).

The present invention further provides polypeptide molecules that comprise the nucleotide sequence of SEQ ID NOs: 2 and 4 as well as those polypeptide molecules encoded by the polynucleotide sequences of SEQ ID NOs: 1, 3, 5-35. A polypeptide molecule comprises an amino acid sequence when the amino acid sequence is at least part of the final amino acid sequence of the polypeptide molecule. In such a fashion, the polypeptide molecule can be only the amino acid sequence or have additional amino acid residues, such as residues that are naturally associated with it or heterologous nucleotide sequences. Such a polypeptide molecule can have one to a few additional amino acids or can comprise many more additional amino acids.

Isolated nucleic acid molecules include, but are not limited to, nucleic acid molecules having a sequence encoding a peptide alone, a sequence encoding a mature peptide and additional coding sequences such as a leader or secretory sequence (e.g., a pre-pro or pro-protein sequence), a sequence encoding a mature peptide with or without additional coding sequences, plus additional non-coding sequences, for example introns and non-coding 5′ and 3′ sequences such as transcribed but untranslated sequences that play a role in, for example, transcription, mRNA processing (including splicing and polyadenylation signals), ribosome binding, gene silencing, RNA polymerization, and/or stability of mRNA, In addition, the nucleic acid molecules may be fused to heterologous marker sequences encoding, for example, a peptide that facilitates purification. Furthermore, isolated nucleic acid molecules of the invention form complexes with polypeptides and optionally perform functions such as RNA polymerization or have terminal transferase activity.

Isolated polypeptides of the invention form complexes with other polypeptides and nucleic acid molecules, including DNA and RNA. Polypeptides and polypeptide complexes of the invention perform functions and/or have enzymatic activity. In one aspect of the invention, polypeptides and polypeptide complexes (which include RNA) perform RNA-dependent RNA polymerization (RdRP) and/or have terminal transferase activity. In another aspect of the invention, polypeptides and polypeptide complexes (which include RNA) have telomerase activity and/or RdRP functions and/or terminal transferase activity.

Isolated nucleic acid molecules can be in the form in of RNA, such as mRNA or siRNA, or in the form DNA, including cDNA and genomic DNA, which may be obtained, for example, by molecular cloning or produced by chemical synthetic techniques or by a combination thereof (Sambrook and Russell, 2000, Molecular Cloning: A Laboratory Manual, Cold Spring Harbor Press, NY). Furthermore, isolated nucleic acid molecules can also be partially or completely in the form of one or more types of nucleic acid analogs, such as peptide nucleic acid (PNA) (U.S. Pat. Nos. 5,539,082; 5,527,675; 5,623,049; 5,714,331). The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled, for example, from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well known in the art (see, e.g., Corey, “Peptide nucleic acids: expanding the scope of nucleic acid recognition”, Trends Biotechnol. 1997 June; 15(6):224-9, and Hyrup et al., “Peptide nucleic acids (PNA): synthesis, properties and potential applications”, Bioorg Med. Chem. 1996 January; 4(1):5-23). Furthermore, large-scale automated oligonucleotide/PNA synthesis (including synthesis on an array or bead surface or other solid support) can readily be accomplished using commercially available nucleic acid synthesizers, such as the Applied Biosystems (Foster City, Calif.) 3900 High-Throughput DNA Synthesizer or Expedite 8909 Nucleic Acid Synthesis System, and the sequence information provided herein.

The present invention encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes). Furthermore, kits/systems (such as beads, arrays, etc.) that include these analogs are also encompassed by the present invention. For example, PNA oligomers that are based on the polymorphic sequences of the present invention are specifically contemplated. PNA oligomers are analogs of DNA in which the phosphate backbone is replaced with a peptide-like backbone (Lagriffoul et al., Bioorganic & Medicinal Chemistry Letters, 4: 1081-1082 (1994), Petersen et al., Bioorganic & Medicinal Chemistry Letters, 6: 793-796 (1996), Kumar et al., Organic Letters 3(9): 1269-1272 (2001), WO96/04000). PNA hybridizes to complementary RNA or DNA with higher affinity and specificity than conventional oligonucleotides and oligonucleotide analogs. The properties of PNA enable novel molecular biology and biochemistry applications unachievable with traditional oligonucleotides and peptides.

The term “isolated polynucleotide” is not limited to molecules containing only naturally-occurring RNA or DNA, but also encompasses chemically-modified nucleotides and non-nucleotides.

In certain embodiments, the nucleic acid molecules of the invention lack 2-hydroxy (2-OH) containing nucleotides. In certain embodiments nucleic acid molecules do not require the presence of nucleotides having a 2′-hydroxy group for mediating gene silencing and as such, isolated nucleic acid molecules, optionally do not include any ribonucleotides (e.g., nucleotides having a 2′-OH group). Such nucleic acid molecules that do not require the presence of ribonucleotides within the polynucleic molecule to support gene silencing can however have an attached linker or linkers or other attached or associated groups, moieties, or chains containing one or more nucleotides with 2′-OH groups. Optionally, miRNA molecules can comprise ribonucleotides at about 5, 10, 20, 30, 40, or 50% of the nucleotide positions.

As used herein, the term “siRNA” is meant to be equivalent to other terms used to describe nucleic acid molecules that are capable of mediating sequence specific gene silencing or interference, e.g., microRNA (miRNA), double-stranded RNA (dsRNA), interfering RNA (RNAi), short hairpin RNA (shRNA), short interfering oligonucleotide, short interfering nucleic acid, short interfering oligonucleotide, chemically-modified siRNA, post-transcriptional gene silencing RNA (ptgsRNA), and other art-recognized equivalents. As used herein, the term “gene silencing” is meant to describe the downregulation, knock-down, degradation, inhibition, suppression, repression, prevention, or decreased expression of a gene, transcript and/or polypeptide product. Gene silencing and interference also describe the prevention of translation of mRNA transcipts into a polypeptide. Translation is prevented, inhibited, or decreased by degrading mRNA transcipts or blocking mRNA translation.

In other embodiments, siRNA molecules, or precursors thereof, may comprise separate sense and antisense sequences or regions, wherein the sense and antisense regions are covalently linked by nucleotide or non-nucleotide linker molecules, or are alternately non-covalently linked by ionic interactions, hydrogen bonding, van der waals interactions, hydrophobic interactions, and/or stacking interactions.

As used herein the term “antisense RNA” is an RNA strand having a sequence complementary to a target gene mRNA, and thought to induce gene silencing or interference by binding to the target gene mRNA. As used herein the term “Sense RNA” has a sequence complementary to the antisense RNA, and when annealed to its complementary antisense RNA, forms a siRNA.

Non-limiting examples of chemical modifications that are made in an isolated polynucleotide include without limitation phosphorothioate internucleotide linkages, 2-deoxyribonucleotides, 2′-0-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in isolated polynucleotides dramatically increase the serum stability of these compounds.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, e.g., when compared to an all-RNA nucleic acid molecule, the overall activity of the modified nucleic acid molecule can be greater than that of the native molecule due to improved stability and/or delivery of the molecule. Unlike native polynucleotides, chemically-modified polynucleotides can also minimize the possibility of activating interferon activity in humans.

Modified nucleotides present in isolated polynucleotide molecules, comprise modified nucleotides having properties or characteristics similar to naturally occurring ribonucleotides. For example, the invention provides nucleic acid molecules including modified nucleotides having a northern conformation (e.g.) northern pseudorotation cycle, see, e.g., Saenger, Principles of Nucleic Acid Structure, Springer-Verlag Ed., 1984). As such, chemically modified nucleotides present in the polynucleotides of the invention, are resistant to nuclease degradation. Non-limiting examples of nucleotides having a northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides. 2′-deoxy-2′-chloro nucleotides, 2′-azido nucleotides, and 2′-0-methyl nucleotides.

A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymidine, e.g., at the Cl position of the sugar.

Additional examples of nucleic acid modifications that improve the binding properties and/or stability of a nucleic acid include the use of base analogs such as inosine, intercalators (U.S. Pat. No. 4,835,263) and the minor groove binders (U.S. Pat. No. 5,801,115). Thus, references herein to nucleic acid molecules include PNA oligomers and other nucleic acid analogs. Other examples of nucleic acid analogs and alternative/modified nucleic acid chemistries known in the art are described in Current Protocols in Nucleic Acid Chemistry, John Wiley & Sons, N.Y. (2002). Isolated nucleic acids of the inventions are comprised of base analogs including, but not limited to, any of the known base analogs of DNA and RNA such as, but not limited to 4-acetylcytosine, 8-hydroxy-N-6-methyladenosine, aziridinylcytosine, pseudoisocytosine, 5-(carboxyhydroxylmethyl)uracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methyl guanine, 1-methyl inosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-Dmannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, 2,6-diaminopurine, and 2′-modified analogs such as, but not limited to 0-methyl, amino-, and fluoro-modified analogs.

The isolated polynucleotides of the invention are modified to enhance stability by modification with nuclease resistant groups, e.g., 2′-amino, 2′-Callyl, 2′-fluoro, 2′-0-methyl, 2′-H. (For a review see Usman and Cedergren, TIBS 17:34, 1992; Usman, et al., Nucleic Acids Symp. Ser. 317163, 1994), Isolated polynucleotides are purified by gel electrophoresis using general methods or can be purified by high pressure liquid chromatography and re-suspended in water.

Chemically synthesizing nucleic acid molecules with modifications (base, sugar and/or phosphate) prevents their degradation by serum ribonucleases, which increases their potency. See, e.g., Eckstein, et al., International Publication No, WO 92/07065; Perrault, et al., Nature 344:565, 1990; Pieken, et al., Science 253:314, 1991; Usman and Cedergren, Trends in Biochem. Sci. 17:334, 1992; Usman, et al, International Publication No. WO 93/15187; and Rossi, et al, International Publication No, WO 91/03162; Sproat, U.S. Pat. No. 5,334,711; Gold, et al., U.S. Pat. No, 6,300,074. All of the above references describe various chemical modifications that are made to the base, phosphate and/or sugar moieties of the isolated nucleic acid molecules described herein.

There are several examples in the art describing sugar, base and phosphate modifications that are introduced into isolated nucleic acid molecules of the invention with significant enhancement in their nuclease stability and efficacy. For example, oligonucleotides are modified to enhance stability and/or enhance biological activity by modification with nuclease resistant groups, e.g., T-amino, 2′-C-allyl, 2′-fluoro, 2′-0-methyl, 2′-H, nucleotide base modifications. For a review see Usman and Cedergren; TIBS 17:34, 1992; Usman, et al., Nucleic Acids Symp. Ser. 31:163, 1994; Burgin, et al., Biochemistry 35:14090, 1996. Sugar modification of nucleic acid molecules have been extensively described in the art. See Eckstein, et al., International Publication PCT No. WO 92/07065; Perrault, et al., Nature 344:565-568, 1990; Pieken, et al., Science 253:314-317, 1991; Usman and Cedergren, Trends in Biochem. Sci. 17:334339, 1992; Usman, et al., International Publication PCT No. WO 93/15187; Sproat, U.S. Pat. No. 5,334,711 and Beigelman, et al., J. Biol. Chem., 270:25702, 1995; Beigelman, et al., International PCT publication No. WO 97/26270; Beigelman, et al., U.S. Pat. No. 5,716,824; Usman, et al., U.S. Pat. No. 5,627,053; Woolf, et al., International PCT Publication No. WO 98/13526; Thompson, et al., Karpeisky, et al, Tetrahedron Lett. 39:1131, 1998; Earnshaw and Gait, Biopolymers (Nucleic Acid Sciences) 48:39-55, 1998; Verma and Eckstein, Annu. Rev. Biochem, 67:99-134, 1998; and Burlina, et al, Bioorg. Med. Chem. 5:1999-2010, 1997. Such publications describe general methods and strategies to determine the location of incorporation of sugar, base and/or phosphate modifications and the like into nucleic acid molecules without modulating catalysis. In view of such teachings, similar modifications are used as described herein to modify the polynucleotide molecules of the invention so long as the ability of the polynucleotides to either bind hTERT or to regulate gene silencing in cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity. Therefore, when engineering isolated nucleic acid molecules of the invention, the amount of these internucleotide linkages are minimized. The reduction in the concentration of these linkages lowers toxicity, resulting in increased efficacy and higher specificity of these molecules.

In one embodiment, the invention provides nucleic acid molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, “Nucleic Acid Analogues: Synthesis and Properties, in Modern Synthetic Methods,” VCH, 331-417, 1995, and Mesmaeker, et al, “Novel Backbone Replacements for Oligonucleotides, in Carbohydrate Modifications in Antisense Research,” ACS, 24-39, 1994.

Labeled nucleotides are the preferred form of label since they can be directly incorporated into the nucleic acid molecules during synthesis. Examples of detection labels that can be incorporated into amplified nucleic acids, such as amplified RNA, include nucleotide analogs such as BrdUrd (Hoy and Schimke, Mutation Research 290:217-230 (1993)), BrUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res. 22:3226-3232 (1994)). A preferred nucleotide analog label for RNA molecules is Biotin-14-cytidine-5′-triphosphate. Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Further variants of the nucleic acid molecules including, but not limited to those identified as SEQ ID NOs: 1, 3, 5-35, such as naturally occurring allelic variants (as well as orthologs and paralogs) and synthetic variants produced by mutagenesis techniques, can be identified and/or produced using methods well known in the art. Such further variants can comprise a nucleotide sequence that shares at least 70-80%, 80-85%, 85-90%, 91%, 92%, 93%, 94%, 95%, 96%, 97%, 98%, or 99% sequence identity with a nucleic acid sequence disclosed as SEQ ID NOs: 1, 3, 5-35 (or a fragment thereof). Thus, the present invention specifically contemplates isolated nucleic acid molecule that have a certain degree of sequence variation compared with the sequences of SEQ ID NOs: 1, 3.5-35.

Further variants of the polypeptide molecules including, but not limited to those identified as SEQ ID NOs: 2 and 4, such as naturally occurring allelic variants (as well as orthologs and paralogs) and synthetic variants produced by mutagenesis techniques, can be identified and/or produced using methods well known in the art. Such further variants can comprise an amino acid sequence that shares at least 70-80%, 80-85%, 85-90%, 91%, 92%, 93%, 94%, 95%, 96%, 97%, 98%, or 99% sequence identity with a nucleic acid sequence disclosed as SEQ ID NOs: 2 and 4 (or a fragment thereof). Thus, the present invention specifically contemplates isolated polypeptide molecules that have a certain degree of sequence variation compared with the sequences of SEQ ID NOs: 2 and 4.

The nucleic acids of the invention are routinely made through techniques such as solid phase synthesis. Equipment for such synthesis is sold by several vendors including, for example, Applied Biosystems, (Foster City, Calif.). Any other means for such synthesis known in the art is additionally or alternatively employed. It is well known to use similar techniques to prepare polynucleotides such as the phosphorothioates and alkylated derivatives.

Polynucleotidesare synthesized using protocols known in the art, e.g., as described in Caruthers, et al., Methods in Enzymology 211:3-19, 1992; Thompson, et al., International PCT Publication No. WO 99/54459; Wincott, et al., Nucleic Acids Res. 23:2677-2684, 1995; Wincott, et al., Methods Mol. Bio. 74:59, 1997; Brennan, et al., Biotechnol Bioeng. 61:33-45, 1998; and Brennan, U.S. Pat. No. 6,001,311. Synthesis of RNA follows general procedures as described, e.g., in Usman, et al, J. Am. Chem. Soc. 109:7845, 1987; Scaringe, et al., Nucleic Acids Res. 18:5433, 1990; and Wincott, et al., Nucleic Acids Res. 23:2677-2684, 1995; Wincott, et al., Methods Mol. Bio, 74:59, 1997.

The comparison of sequences and determination of percent identity between two sequences can be accomplished using a mathematical algorithm. (Computational Molecular Biology, Lesk, A. M., ed., Oxford University Press, New York, 1988; Biocomputing: Informatics and Genome Projects, Smith; D. W., ed., Academic Press, New York, 1993; Computer Analysis of Sequence Data, Part 1, Griffin, A. hit, and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). In a preferred embodiment, the percent identity between two amino acid sequences is determined using the Needleman and Wunsch algorithm (J. Mol. Biol. (48):444-453 (1970)) which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6.

In yet another preferred embodiment, the percent identity between two nucleotide sequences is determined using the GAP program in the GCG software package (Devereux, J., et al., Nucleic Acids Res. 12(1):387 (1984)), using a NWSgapdna. CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Myers and W. Miller (CABIOS, 4:11-17 (1989)) which has been incorporated into the ALIGN program (version 2.0), using a PAM 120 weight residue table, a gap length penalty of 12, and a gap penalty of 4.

The nucleotide and amino acid sequences of the present invention can further be used as a “query sequence” to perform a search against sequence databases to, for example, identify, other family members or related sequences. Such searches can be performed using the NBLAST and BLAST programs (version 2.0) of Altschul, et al. (J. Mol. Biol. 215; 403-10 (1990)), BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (Nucleic Acids Res. 25(17):3389-3402 (1997)). When utilizing BLAST and gapped BLAST programs, the default parameters of the respective programs (BLAST and NBLAST) can be used. In addition to BLAST, examples of other search and sequence comparison programs used in the art include, but are not limited to, FASTA (Pearson, Methods Mol. Biol. 25, 365-389 (1994)) and KERR (Dufresne et al., Nat Biotechnol 2002 December; 20(12): 1269-71). For further information regarding bioinformatics techniques, see Current Protocols in Bioinformatics, John Wiley & Sons, Inc., N.Y.

Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Nucleic acid sequences can be aligned by visual inspection, or by using sequence alignment software. For example, MEGALIGN™ (DNASTAR, Madison, Wis., 1997) sequence alignment software, using default parameters for the Clustal algorithm, can be used to align polynucleotides. In this method, sequences are grouped into clusters by examining the distance between all pairs. Clusters are aligned as pairs, then as groups.

### Therapeutic Methods

The invention provides methods of treating disease by administering to a subject in need thereof a composition of the invention or a TERT polypeptide, or alternatively, an inhibitor of the RdRP activity of a composition of the invention or a TERT polypeptide. Contemplated diseases are caused by the inappropriate and/or pathological deletion, silencing, decreased accessibility, function- or activity-blocking mutation, methylation, decreased dosage, decreased copy number, or decreased abundance of a product of a gene. Alternatively, or in addition, contemplated diseases are caused by the undesired, inappropriate, and/or pathological overexpression, activation, increased accessibility, demethylation, increased copy number, increased dosage, function- or activity-enhancing mutation, or increased abundance of a product of a gene.

Compositions and inhibitors of compositions of the invention are administered in a therapeutically effective amount to subjects in need thereof. Subjects are identified through a number of methods by a medical professional or by one of ordinary skill in the art, e.g. a researcher conducting a study. Subjects are identified as having a disorder caused by a disease of the invention by the presentation of symptoms and followed by genetic confirmation.

Genetic confirmation includes, but is not limited to, amplification of a polynucleotide sequence from one gene to confirm abnormal gene dosage, a mutation, or the absence of a gene by methods known in the art. Alternatively, a genetic sample is probed using a polynucleotide or polypeptide probe complementary to a polynucleotide or polypeptide sequence of a target gene using methods known in the art (e.g. Western, Northern, Southern Blotting and Immunoprecipitation). The use of probes to highlight target sequences also allows to quantification and identification of genes, mRNA transcripts, and polypeptide gene products. Furthermore, genetic confirmation includes karyotyping to confirm the presence or absence as well as number of chromosomes carried by any particular subject. Karytyping also reveals abnormalities including, but not limited to, chromosomal deletions (encompassing complete and partial gene deletions) and translocations.

A therapeutically effective amount of a composition of the invention is an amount of a TERT subunit, TERT-RMRP complex, or TERT-RNA complex having RdRP activity, or a combination thereof, that when administered to a subject, results in the silencing, or decreased expression, of at least one gene or mRNA transcript. The effectiveness of administration of a pharmaceutical composition of the invention is measured, in this embodiment, by testing a subject, e.g. biopsied tissue or a bodily fluid, for decreased gene expression using art-recognized methods.

Alternatively, or in addition, a therapeutically effective amount of a composition of the invention is an amount of a TERT subunit, TERT-RMRP complex, or TERT-RNA complex having RdRP activity, or a combination thereof, that when administered to a subject, results in the activation, or increased expression or abundance, of at least one gene or mRNA transcript. The effectiveness of administration of a pharmaceutical composition of the invention is measured, in this embodiment, by testing a subject, e.g. biopsied tissue or a bodily fluid, for increased gene expression using art-recognized methods.

Alternatively, or in addition, a pharmaceutically effective amount of a composition of the invention is an amount of a TERT subunit, TERT-RMRP complex, or TERT-RNA complex having RdRP activity, or a combination thereof, that prevents, inhibits the occurrence or reoccurrence of, treats, or alleviates a sign or symptom (to some extent) of a disorder. As used herein, the term “treat” is meant to describe a process by which a sign or symptom of a disorder is eliminated. Alternatively, or in addition, a disorder, which can occur in multiple tissues or at multiple gene loci, is treated if the disorder is eliminated within at least one of the multiple tissues or gene expression is affected in at least one of the multiple gene loci.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder, which can occur in multiple tissues or at multiple gene loci, is alleviated if the severity of the cancer is decreased within at least one of the multiple tissues or gene expression is affected in at least one of the multiple gene loci.

As used herein, the term “severity” is meant to describe the exacerbation of a sign or symptom. Alternatively, or in addition, increasing severity is meant to describe the increased deviation of gene expression away from the expected average gene expression level calculated from gene expression studies of comparable healthy individuals.

In one aspect of the invention, a therapeutically effective amount of a composition of the invention is an amount of a TERT subunit, TERT-RMRP complex, or TERT-RNA complex having RdRP activity, or a combination thereof, that provides a preventative benefit to the subject. As used herein, the term “preventative benefit” is meant to describe a delay in the development or decrease of the severity of a sign or symptom of a disorder.

The pharmaceutically effective dose depends on the type of disease, the composition used, the route of administration, the individual and physical characteristics of the subject wider consideration (for example, age, gender, weight, diet, smoking-habit, exercise-routine, genetic background, medical history, hydration, blood chemistry), concurrent medication, and other factors that those skilled in the medical arts will recognize.

Generally, an amount from about 0.01 mg/kg and 25 mg/kg body weight/day of active ingredients is administered dependent upon potency of the composition. In alternative embodiments dosage ranges include, but are not limited to, 0.01-0.1 mg/kg, 0.01-1 mg/kg, 0.01-10 mg/kg, 0.01-20 mg/kg, 0.01-30 mg/kg, 0.01-40 mg/kg, 0.01-50 mg/kg, 0.01-60 mg/kg, 0.01-70 mg/kg, 0.01-80 mg/kg, 0.01-90 mg/kg, 0.01-100 mg/kg, 0.01-150 mg/kg, 0.01-200 mg/kg, 0.01-250 mg/kg, 0.01-300 mg/kg, 0.01-500 mg/kg, and all ranges and points in between. In alternative embodiments dosage ranges include, but are not limited to, 0.01-1 mg/kg, 1-10 mg/kg, 10-20 mg/kg, 20-30 mg/kg, 30-40 mg/kg, 40-50 mg/kg, 50-60 mg/kg, 60-70 mg/k/0-80 mg/kg, 80-90 mg/kg, 90-100 mg/kg, 100-150 mg/kg, 150-200 mg/kg, 200-300 mg/kg, 300-500 mg/kg, and all ranges and points in between.

Exemplary disorders that are treated by the methods of the invention include those disorders caused by the undesired or overexpression of a gene. Moreover, disorders in which a gene is present in more than the expected or desired two copies due to chromosomal abnormalities or other causes, this method is used to partially silence gene expression such that gene dosage levels are normal. Alternatively, or in addition, the disorder is caused by the undesired or overexpression of at least one gene. Moreover, the disorder is caused by the undesired or overexpression of one or more gene(s). Nonlimiting examples of disorders caused by undesired or overexpression of a gene include, cell proliferative disorders (e.g. cancer, neoplastic and inflammatory disorders), autoimmune disorders (e.g. Multiple Sclerosis (MS) and Coeliac/Celiac disease), gene/chromosome duplication disorders (Down Syndrome/Trisomy 21 and Kleinfelter Syndrome/XXY), metabolic disorders and stem cell disorders.

Exemplary disorders that are treated by the methods of the invention include those disorders caused by the inappropriate deactivation of a gene. Moreover, disorders in which one copy of a gene is deleted are treated as having one copy deactivated, or are inappropriately deactivated, and therefore, are treated using this method to increase the dosage effect of the working copy. Alternatively, or in addition, disorders in which a mutation has made one copy of a gene non-functional are treated using this method to boost the gene dosage from the functional copy as a compensatory mechanism. Furthermore, disorders in which one copy of a gene is not functional, and/or the other copy is developmentally silenced, e.g. in the case of X-chromosome in females, this method is used to activated the silenced copy to compensate for the non-functional or mutated copy. Alternatively, or in addition, the disorder is caused by the inappropriate deactivation of at least one gene. Moreover, the disorder is caused by the inappropriate deactivation of one or more gene(s). Nonlimiting general examples of disorders caused by the inappropriate deactivation of a gene include, stein cell disorders (e.g. bone marrow failure), cell proliferative disorders (e.g. cancer, neoplastic and inflammatory disorders), metabolic disorders, immunological disorders (immunodeficiency), and developmental disorders. Nonlimiting specific examples of disorders caused by inappropriate deactivation of a gene include, 1p36 syndrome, 22ql 1.2 deletion syndrome, Achondraplasia, Angelman syndrome (AS), Amyotrophic lateral sclerosis (ALS), Canavan disease, Cartilage-Hair Hypoplasia, Charcot-Marie-Tooth disease(s), Cri du Chat disease, Duchenne muscular dystrophy, ectodermal dysplasia, Prader-Willi Syndrome, and Turner Syndrome.

For all therapeutic methods, the full range of contemplated diseases can be found within the Online Mendelian Inheritance in Man™ (OMIM™). This database is a catalog of human genes and genetic disorders authored and edited by Dr. Victor A. McKusick and colleagues at Johns Hopkins University and elsewhere. The database has been developed for the world wide web by NCBI (National Center for Biotechnology Information) and is freely available to the public.

### Pharmaceutical Compositions

The invention provides a composition including a TERT subunit, TERT-RMRP complex, or TERT-RNA complex having RdRP activity, or a combination thereof, and a pharmaceutically acceptable carrier. Pharmaceutically acceptable carriers are covalently or non-covalently bound, admixed, encapsulated, conjugated, operably-linked, or otherwise associated with the composition such that the pharmaceutically acceptable carrier increases the cellular uptake, stability, solubility, half-life, binding efficacy, specificity, targeting, distribution, absorption, or renal clearance of the composition. Alternatively, or in addition, the pharmaceutically acceptable carrier increases or decreases the immunogenicity of the composition. Furthermore, the pharmaceutically acceptable carrier is capable to increasing the cytotoxicity of the composition with respect to the targeted cells or tissues.

Alternatively, or in addition, pharmaceutically acceptable carriers are salts (for example, acid addition salts, e.g., salts of hydrochloric, hydrobromic, acetic acid, and benzene sulfonic acid), esters, salts of such esters, or any other compound which, upon administration to a subject, are capable of providing (directly or indirectly) the biologically active compositions of the invention. As such, the invention encompasses prodrugs, and other bioequivalents. As used herein, the term “prodrug” is meant to describe, a pharmacological substance that is administered in an inactive (or significantly less active) form. Once administered, the prodrug is metabolised in vivo into an active metabolite. Pharmaceutically acceptable carriers are alternatively or additionally diluents, excipients, adjuvants, emulsifiers, buffers, stabilizers, and/or preservatives.

Pharmaceutically acceptable carriers of the invention are delivery systems/mechanisms that increase uptake of the composition by targeted cells. For example, pharmaceutically acceptable carriers of the invention are viruses, recombinant viruses, engineered viruses, viral particles, replication-deficient viruses, liposomes, cationic lipids, anionic lipids, cationic polymers, polymers, hydrogels, micro- or nano-capsules (biodegradable), micropheres (optionally bioadhesive), cyclodextrins, plasmids, mammalian expression vectors, proteinaceous vectors, or any combination of the preceeding elements (see, O'Hare and Normand, International PCT Publication No. WO 00/53722; U.S. Patent Publication 2008/0076701). Moreover, pharmaceutically acceptable carriers that increase cellular uptake can be modified with cell-specific proteins or other elements such as receptors, ligands, antibodies to specifically target cellular uptake to a chosen cell type.

In one aspect, the active compounds are prepared with pharmaceutically acceptable carriers that will protect the composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Examples of materials which can form hydrogels include polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-epsilon.-caprolactone, polyanhydrides; polyphosphazines, poly(vinyl alcohols), poly(alkylene oxides) particularly poly(ethylene oxides), poly(allylamines)(PAM), poly(acrylates), modified styrene polymers such as poly(4-aminomethylstyrene), pluronic polyols, poloxamers, poly(uronic acids), poly(vinylpyrrolidone) and copolymers of the above, including graft copolymers.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Pharmaceutically acceptable carriers are cationic lipids that are bound or associated with compositions of the invention. Alternatively, or in addition, compositions are encapsulated or surrounded in cationic lipids, e.g. liposomes, for in vivo delivery. Exemplary cationic lipids include, but are not limited to, N41-(2,3-dioleoyloxy)propyli-N,N,N-trimethylammonium chloride (DOTMA); (trimethylammonium)propane (DOTAP), 1,2-bis(dimyrstoyloxy)-3-3-(trimethylammonia)propane (DMTAP); 1,2-dimyristyloxypropyl-3-dimethylhydroxyethylammonium bromide (DMRIE); dimethyldioctadecylammonium bromide (DDAB); 3-(N—(N′,N′-dimethylaminoethane)carbamoyl)cholesterol (DC-Chol); 3.beta.-[N′,N′-diguanidinoethyl-aminoethane)carbamoyl cholesterol (BGTC); 2-(2-(3-(bis(3-aminopropyl)amino)propylamino)acetamido)-N,N-ditetradecyla-cetamide (PR209120); pharmaceutically acceptable salts thereof, and mixtures thereof. Further examplary cationic lipids include, but are not limited to, 1,2-dialkenoyl-sn-glycero-3-ethylphosphocholines (EPCs), such as 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, pharmaceutically acceptable salts thereof, and mixtures thereof.

Exemplary polycationic lipids include, but are not limited to, tetramethyltetrapalmitoyl spermine (TMTPS), tetramethyltetraoleyl spermine (TMTOS), tetramethlytetralauryl spermine (TMTLS), tetramethyltetramyristyl spermine (TMTMS), tetramethyldioleyl spermine (TMDOS), pharmaceutically acceptable salts thereof, and mixtures thereof. Further examplary polycationic lipids include, but are not limited to, 2,5-bis(3-aminopropylamino)-N-(2-(dioctadecylamino)-2-oxoethyl)pentanamid-e (DOGS); 2,5-bis(3-aminopropylamino)-N-(2-(di(Z)-octadeca-9-dienylamino)-2-oxoethyl) pentanamide (DOGS-9-en); 2,5-bis(3-aminopropylamino)-N-(2-(di(9Z,127)-octadeca-9,12-dienylamino)-2-oxoethyl)pentanamide (DLinGS); 3-beta-(N.sup.4-(N.sup.1, N.sup.8-dicarbobenzoxyspermidine)carbamoyl)chole-sterol (GL-67); (9Z,9^{y}Z)-2-(2,5-bis(3-aminopropylamino)pentanamido)propane-1,3-diyl-dioct-adec-9-enoate (DOSPER); 2,3-dioleyloxy-N-[2(sperminecarboxamido)ethyl]-N,N-dimethyl-1-propanamini-urn trifluoroacetate (DOSPA); pharmaceutically acceptable salts thereof, and mixtures thereof.

Examples of cationic lipids are described in U.S. Pat. Nos. 4,897,355; 5,279,833; 6,733,777; 6,376,248; 5,736,392; 5,334,761; 5,459,127; 2005/0064595; U.S. Pat. Nos. 5,208,036; 5,264,618; 5,279,833; 5,283,185; 5,753,613; and 5,785,992; each of which is incorporated herein in its entirety.

Pharmaceutically acceptable carriers of the invention also include non-cationic lipids, such as neutral, zwitterionic, and anionic lipids. Exemplary non-cationic lipids include, but are not limited to, 1,2-Dilauroyl-sn-glycerol (DLG); 1,2-Dimyristoyl-snglycerol (DMG); 1,2-Dipalmitoyl-sn-glycerol (DPG); 1,2-Distearoyl-sn-glycerol (DSG); 1,2-Dilauroyl-sn-glycero-3-phosphatidic acid (sodium salt; DLPA); 1,2-Dimyristoyl-sn-glycero-3-phosphatidic acid (sodium salt; DMPA); 1,2-Dipalmitoyl-sn-glycero-3-phosphatidic acid (sodium salt; DPPA); 1,2-Distearoyl-sn-glycero-3-phosphatidic acid (sodium salt; DSPA); 1,2-Diarachidoyl-sn-glycero-3-phosphocholine (DAPC); 1,2-Dilauroyl-sn-glycero-3-phosphocholine (DLPC); 1,2-Dimyristoyl-sn-glycero-3-phosphocholine (DMPC); 1,2-Dipalmitoyl-sn-glycero-O-ethyl-3-phosphocholine (chloride or vitiate; DPePC); 1,2-Dipalmitoyl-sn-glycero-3-phosphocholine (DPPC); 1,2-Distearoyl-sn-glycero-3-phosphocholine (DSPC); 1,2-Dilauroyl-sn-glycero-3-phosphoethanolamine (DLPE); 1,2-Dimyristoyl-sn-glycero-3-phosphoethanolamine (DMPE); 1,2-Dipalmitoyl-sn-glycero-3-phosphoethanolamine (DPPE); 1,2-Distearoyl-sn-glycero-3-phosphoethanolamine (DSPE); 1,2-Dilauroyl-sn-glycero-3-phosphoglycerol (sodium salt; DLPG); 1,2-Dimyristoyl-sn-glycero-3-phosphoglycerol (sodium salt; DMPG); 1,2-Dimyristoyl-sn-glycero-3-phospho-sn-1-glycerol (ammonium salt; DMP-sn-1-G); 1,2-Dipalmitoyl-sn-glycero-3-phosphoglycerol (sodium salt; DPPG); 1,2-Distearoyl-sn-glycero-3-phosphoglycero (sodium salt; DSPG); 1,2-Distearoyl-sn-glycero-3-phospho-sn-1-glycerol (sodium salt; DSP-sn-1-G); 1,2-Dipalmitoyl-sn-glycero-3-phospho-L-serine (sodium salt; DPP S); 1-Palmitoyl-2-linoleoyl-sn-glycero-3-phosphocholine (PLinoPC); 1-Palmitoyl-2-oleoyl-sn-glycero-3-phosphocholine (POPC); 1-Palmitoyl-2-oleoyl-sn-glycero-3-phosphoglycerol (sodium salt; POPG); 1-Palmitoyl-2-oleoyl-sn-glycero-3-phosphoglycerol (sodium salt; POPG); 1-Palmitoyl-2-oleoyl-sn-glycero-3-phosphoglycerol (ammonium salt; POPG); 1-Palmitoyl-2-4o-sn-glycero-3-phosphocholine (P-lyso-PC); 1-Stearoyl-2-lyso-sn-glycero-3-phosphocholine (S-lyso-PC); and mixtures thereof. Further exemplary non-cationic lipids include, but are not limited to, polymeric compounds and polymer-lipid conjugates or polymeric lipids, such as pegylated lipids, including polyethyleneglycols, N-(Carbonyl-methoxypolyethylenealycol-2000)-1,2-dimyristoyl-sn-glycero-3-phosphoethanolamine (sodium salt; DMPE-MPEG-2000); N-(Carbonyl-methoxypolyethyleneglycol-5000)-1,2-dimyristoyl-sn-glycero-3-phosphoethanolamine (sodium salt; DMPE-MPEG-5000); N—(Carbonyl-methoxypolyethyleneglycol 2000)-1,2-dipalmitoyl-sn-glycero-3-phosphoethanolamine (sodium salt; DPPE-MPEG-2000); N-(Carbonyl-methoxypolyethyleneglycol 5000)-1,2-dipalmitoyl-sn-glycero-3-phosphoethanolamine (sodium salt; DPPE-MPEG-5000); N-(Carbonyl-methoxypolyethyleneglycol 750)-1,2-distearoyl-sn-glycero-3-phosphoethanolamine (sodium salt; DSPE-MPEG-750); N-(Carbonyl-methoxypolyethyleneglycol 2000)-1,2-distearoyl-sn-glycero-3-phosphoethanolamine (sodium salt; DSPE-MPEG-2000); N-(Carbonyl-methoxypolyethyleneglycol 5000)-1,2-distearoyl-sn-glycero-3-phosphoethanolamine (sodium salt; DSPE-MPEG-5000); sodium cholesteryl sulfate (SCS); pharmaceutically acceptable salts thereof, and mixtures thereof. Examples of non-cationic lipids include, but are not limited to, dioleoylphosphatidylethanolamine (DOPE), diphytanoylphosphatidylethanolamine (DPhPE), 1,2-Dioleoyl-sn-Glycero-3-Phosphocholine (DOPC), 1,2-Diphytanoyl-sn-Glycero-3-Phosphocholine (DPhPC), cholesterol, and mixtures thereof.

Pharmaceutically-acceptable carriers of the invention further include anionic lipids. Exemplary anionic lipids include; but are not limited to, phosphatidylserine, phosphatidic acid, phosphatidylcholine, platelet-activation factor (PAF), phosphatidylethanolamine, phosphatidyl-DL-glycerol, phosphatidylinositol, phosphatidylinositol (pi(4)p, pi(4,5)p2), cardiolipin (sodium salt), lysophosphatides, hydrogenated phospholipids, sphingolipids, gangliosides, phytosphingosine, sphinganines, pharmaceutically acceptable salts thereof, and mixtures thereof.

Supplemental or complementary methods for delivery of nucleic acid molecules for use herein are described, e.g., in Akhtar, et al., Trends Cell Bio. 2:139, 1992; Delivery Strategies for Antisense Oligonucleotide Therapeutics, ed. Akhtar, 1995; Maurer, et al., Mol. Membr, Biol. 16:129-140, 1999; Hofland and Huang, Handb. Exp. Pharmacol. 137:165-192, 1999; and Lee, et al., ACS Symp. Ser. 752:184-192, 2000. Sullivan, et al., international PCT Publication No. WO 94/02595, further describes general methods for delivery of enzymatic nucleic acid molecules. These protocols can be utilized to supplement or complement delivery of virtually any composition of the invention.

Pharmaceutical compositions are administered locally and/or systemically. As used herein, the term “local administration” is meant to describe the administration of a pharmaceutical composition of the invention to a specific tissue or area of the body with minimal dissemination of the composition to surrounding tissues or areas. Locally administered pharmaceutical compositions are not detectable in the general blood stream when sampled at a site not immediate adjacent or subjacent to the site of administration.

As used herein the term “systemic administration” is meant to describe in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes exposes the compositions to an accessible diseased tissue. The rate of entry of a drug into the circulation has been shown to be a function of molecular weight or size. The use of a liposome or other drug carrier comprising the compounds of the instant disclosure can potentially localize the drug, e.g., in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

A pharmaceutically acceptable carrier is chosen to be compatible with its intended route of administration. Examples of routes of administration include parenteral, e.g., intravenous, intradermal, subcutaneous, oral (e.g., inhalation or insufflation), transdermal (topical), transmucosal, transopthalmic, tracheal, intranasal, epidermal, intraperitoneal, intraorbital, intraarterial, intracapsular, intraspinal, imrastemal, intracranial, intrathecal, intraventricular, and rectal administration. Alternatively, or in addition, compositions of the invention are administered non-parentally, for example, orally. Alternatively, or further in addition, compositions of the invention are administered surgically, for example, as implants or biocompatible polymers.

Pharmaceutical compositions are administered via injection or infusion, e.g. by use of an infusion pump. Direct injection of the nucleic acid molecules of the invention, is performed using standard needle and syringe methodologies, or by needle-free technologies such as those described in Conry et al., Clin, Cancer Res, 5:2330-2337, 1999 and Barry et al., International PCT Publication No. WO 99/31262.

Solutions or suspensions used for parenteral, intradermal, or subcutaneous application can include the following components: a sterile diluent such as water for injection; saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof.

The pharmaceutical compositions are in the form of a sterile injectable aqueous or oleaginous suspension. This suspension is formulated according to the known art using those suitable dispersing or wetting agents and suspending agents that have been mentioned above. The sterile injectable preparation is a sterile injectable solution or suspension in a non-toxic parentally acceptable diluent or solvent, e.g., as a solution in 1,3-butanediol. Exemplary acceptable vehicles and solvents are water, Ringer's solution and isotonic sodium chloride solution. In addition, sterile, fixed oils are conventionally employed as a solvent or suspending medium. For this purpose, any bland fixed oil is employed including synthetic mono- or diglycerides. In addition, fatty acids such as oleic acid are used in the preparation of injectables.

Sterile injectable solutions can be prepared by incorporating the composition in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. Compositions containing nucleic acid molecules with at least one 2′-0-methoxyethyl modification are used when formulating compositions for oral administration. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Exemplary penetrants for transdermal administration include, but are not limited to, lipids, liposomes, fatty acids, fatty acid, esters, steroids, chelating agents, and surfactants. Preferred lipids and liposomes of the invention are neutral, negative, or cationic. Compositions are encapsulated within liposomes or form complexes thereto, such as cationic liposomes.

Alternatively, or in addition, compositions are complexed to lipids, such as cationic lipids. Compositions prepared for transdermal administration are provided by iontophoresis. Such penetrants are generally known in the art, and include, for example, for transmucosal administration, detergents, bile salts, and fusidic acid derivatives.

Transmucosal administration can be accomplished through the use of nasal sprays or suppositories. For transdermal administration, the active compounds are formulated into patches, ointments, lotions, salves, gels, drops, sprays, liquids, powders, or creams as generally known in the art.

Pharmaceutical compositions of the invention are administered systemically and are intended to cross the blood-brain barrier to contact cells of the central nervous system. Alternatively, or in addition, pharmaceutical compositions are administered intraspinally by, for example, lumbar puncture, or intracranially, e.g. intrathecally or intraventricularly. By the preceding routes, pharmaceutical compositions are introduced directly into the cerebral spinal fluid. Nonlimiting examples of agents suitable for formulation with the nucleic acid molecules of the invention, particularly for targeting nervous system tissues, include: P-glycoprotein inhibitors (such as Pluronic P85), which can enhance entry of drugs into the CNS (Jolliet-Riant and Tillement, Fundam. Clin. Pharmacol. 13:16-26, 1999); biodegradable polymers, such as poly (DL-lactidecoglycolide) microspheres for sustained release delivery after intracerebral implantation (Emerich, D. F., et al., Cell Transplant 8:47-58, 1999) (Alkermes, Inc. Cambridge, Mass.); and loaded nanoparticles, such as those made of polybutylcyanoacrylate, which can deliver drugs across the blood brain barrier and can alter neuronal uptake mechanisms (Prog. Neuropsychopharmacol Biol. Psychiatry 23:941-949, 1999). Other non-limiting examples of delivery strategies for the nucleic acid molecules of the instant disclosure include material described in Boado, et al., J. Pharm. Sci. 87:1308-1315, 1998; Tyler, et al., FEBS Lett. 421:280-284, 1999; Pardridge, et al, PNAS USA. 92:5592-5596, 1995; Boado, Adv. Drug Delivery Rev. 15:73-107, 1995; Aldrian-Herrada, et al., Nucleic Acids Res. 26:4910-4916, 1998; and Tyler, et al., PNAS USA. 96:7053-7058, 1999.

The compositions of the invention are also administered in the form of suppositories, e.g., for rectal administration of the drug. These compositions are prepared by mixing the drug with a suitable non-irritating excipient that is solid at ordinary temperatures but liquid at the rectal temperature and will therefore melt in the rectum to release the drug. Such materials include cocoa butter and polyethylene glycols.

Aqueous suspensions contain the active materials in admixture with excipients suitable for the manufacture of aqueous suspensions. Such excipients are suspending agents, e.g., sodium carboxymethylcellulose, methylcellulose, hydropropyl methylcellulose, sodium alginate, polyvinylpyrrolidone, gum tragacanth and gum acacia; dispersing or wetting agents can be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, e.g., polyethylene sorbitan monooleate. The aqueous suspensions also contain one or more preservatives, e.g., ethyl, or n-propyl hydroxybenzoate, one or more coloring agents, one or more flavoring agents, and one or more sweetening agents, such as sucrose or saccharin.

Oily suspensions are formulated by suspending the active ingredients in a vegetable oil, e.g., arachis oil, olive oil, sesame oil or coconut oil, or in a mineral oil such as liquid paraffin. The oily suspensions contain a thickening agent, e.g., beeswax, hard paraffin or cetyl alcohol. Sweetening agents and flavoring agents are added to provide palatable oral preparations. These compositions are preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, e.g., sweetening, flavoring and coloring agents, are also present.

Pharmaceutical compositions of the invention are in the form of oil-in-water emulsions. The oily phase is a vegetable oil or a mineral oil or mixtures of these. Suitable emulsifying agents are naturally-occurring gums, e.g., gum acacia or gum tragacanth, naturally-occurring phosphatides, e.g., soy bean, lecithin, and esters or partial esters derived from fatty acids and hexitol, anhydrides, e.g., sorbitan monooleate, and condensation products of the said partial esters with ethylene oxide, e.g., polyoxyethylene sorbitan monooleate. The emulsions also contain sweetening and flavoring agents.

In a preferred aspect, the pharmaceutically acceptable carrier can be a solubilizing carrier molecule. More preferably, the solubilizing carrier molecule can be Poloxamer, Povidone K17, Povidone K12, Tween 80, ethanol, Cremophor/ethanol, Lipiodol, polyethylene glycol (PEG) 400, propylene glycol, Trappsol, alpha-cyclodextrin or analogs thereof beta-cyclodextrin or analogs thereof and gamma-cyclodextrin or analogs thereof.

The invention also provides compositions prepared for storage or administration. Acceptable carriers or diluents for therapeutic use are well known in the pharmaceutical art, and are described, e.g., in Remington's Pharmaceutical Sciences, Mack Publishing Co., A. R. Gennaro Ed., 1985. For example, preservatives, stabilizers, dyes and flavoring agents are provided. These include sodium benzoate, sorbic acid and esters of p-hydroxybenzoic acid. In addition, antioxidants and suspending agents are used.

### Screening Methods

The invention provides methods of screening for agonists, antagonists, and inverse agonists of the activity of a complex comprising a TERT polypeptide or fragment thereof and a RMRP. Alternatively, or in addition, the invention provides methods of identifying agonists, antagonists, and inverse agonists of the activity of a complex comprising a TERT polypeptide or fragment thereof and a RMRP. Further in the alternative or further in addition, the invention provides methods of determining whether a test compound is an agonist, antagonist, or inverse agonist of the activity of a complex comprising a TERT polypeptide or fragment thereof and a RMRP.

As used herein, the term “agonist” is meant to describe a substance or compound that contacts a complex comprising a TERT polypeptide or fragment thereof and a RMRP and activates, induces, enhances, or potentiates RdRP. Subtypes of agonists are further encompassed by the methods of the invention. As used herein, the term “inverse agonist” is meant to describe a substance or compound which contacts a complex comprising a TERT polypeptide or fragment thereof and a RMRP and activates, induces, enhances, or potentiates RdRP and reverses constitutive activity. Inverse agonists exert the opposite pharmacological effect of an agonist.

In one aspect of the invention, one or more substances or compounds work in combination to activate a complex comprising a TERT polypeptide or fragment thereof and a RMRP and activates, induces, enhances, or potentiates RdRP. As used herein, the term “co-agonist” is meant to describe a substance or compound that works with other co-agonists to activate RdRP. In another aspect of the invention, one or more substances, compounds, or co-agonists, work synergistically to activate a complex comprising a TERT polypeptide or fragment thereof and a RMRP and activates, induces, enhances, or potentiates RdRP.

As used herein, the term “antagonist” is meant to describe a substance or compound that inhibits, blocks, decreases, prevents, diminishes, silences, deactivates, or interrupts RdRP activation by agonists. In one aspect of the invention, one or more substances or compounds work in combination to inhibit a complex comprising a TERT polypeptide or fragment thereof and a RMRP and activates, induces, enhances, or potentiates RdRP. As used herein, the term “co-antagonist” is meant to describe a substance or compound that works with other co-antagonists to inhibit RdRP. In another aspect of the invention, one or more substances, compounds, or co-antagonists, work synergistically to inhibit a complex comprising a TERT polypeptide or fragment thereof and a RMRP and activates, induces, enhances, or potentiates RdRP.

The invention also provides methods of identifying selective agonists. As used herein the ter “selective agonist” is meant to describe an agonist that is selective for one TERT-RNA complex. For instance, the agonist is selective for the TERT-RMRP complex, but not for other TERT-RNA complexes. A selective agonist can additionally be of any of the aforementioned types of agonists.

Similarly, the invention provides methods of screening for enhancers and inhibitors of the formation of a complex comprising a TERT polypeptide or fragment thereof and a RMRP. Alternatively, or in addition, the invention provides methods of identifying enhancers and inhibitors of the formation of a complex comprising a TERT polypeptide or fragment thereof and a RMRP. Further in the alternative or further in addition, the invention provides methods of determining whether a test compound is an enhancer or an inhibitor of the formation of a complex comprising a TERT polypeptide or fragment thereof and a RMRP.

As used herein, the term “enhancer” is meant to describe a substance or compound that when brought into contact with a TERT polypeptide, a RMRP, or both, increases the amount of complex formation compared to the amount of complex formation observed in the absence of this substance or compound. In certain aspects of the invention, an enhancer potentiates or catalyzes complex formation by bringing the TERT polypeptide and RMRP in closer physical proximity, by sequestering or removing an inhibitor of complex formation, by lowering the energy required for complex formation, by stabilizing the complex, or by preventing the degradation of the RMRP or TERT until the complex is formed.

As used herein, the term “inhibitor” is meant to describe a substance or compound that when brought into contact with a TERT polypeptide, a RMRP, or both, decreases the amount of complex formation compared to the amount of complex formation observed in the absence of this substance or compound. In one aspect of the invention, an inhibitor prevents or reverses complex formation by antagonizing the activity of an enhancer. In another aspect of the invention, an inhibitor prevents or reverses complex formation by destabilizing the complex, degrading the RMRP or TERT elements of the complex, competitively binding either the TERT or RMRP elements, sterically hindering complex formation, increasing the energy barrier to complex formation, or altering the conformation of a binding motif.

The invention provides methods of increasing gene silencing in a cell including the steps of overexpressing in that cell a TERT polypeptide, a RMRP, or both. Conversely, the invention provides methods of decreasing gene silencing in a cell including the steps of inhibiting or decreasing the expression or activity in that cell of a TERT polypeptide, a RMRP, or both. As used herein, the term “gene silencing” is meant to describe a process by which the transcription or translation of a gene or gene product is temporarily or permanently inhibited, prevented, decreased, diminished or eliminated. As used herein, the term “expression” of a TERT polypeptide, a RMRP, or both is meant to describe the transcription or translation of mRNA or polypeptide sequences that encode TERT, RMRP, or both. As used herein, the term “activity” of a TERT polypeptide, a RMRP, or both is meant to describe the RdRP activity of a TERT polypeptide or TERT-RMRP complex. Furthermore, the term “activity” is meant to describe the ability of a TERT polypeptide to form a complex with RMRP.

The invention provides methods of treating disease. In one aspect, the disease to be treated is caused by undesired or overexpression of a gene and the subject having this disease is treated by administering a composition of the invention, which includes either a TERT-RNA or TERT-RMRP complex, or a TERT polypeptide. As used herein the terms “undesired” and/or “overexpression” are meant to describe excessive or inappropriate gene dosages. In one aspect, a particular gene is transcribed such that the mRNA or polypeptide encoding either a functional RNA or protein is over-abundant, having a deleterious consequence for the subject. In another aspect, a gene is present in more than the expected copy-number. For instance, with respect to sex chromosomes, an individual is XXY, or with respect to autosomes (diploid chromosomes, not X or Y), an individual is trisomy 21 due to a duplication, translocation, or improper chromosome separation event during cell division. In a third aspect, undesired gene expression occurs when a gene that should be silenced or inexcusable to transcriptional machinery, for instance, at a particular developmental stage, is expressed.

In a contrasting aspect, the disease to be treated is caused by the inappropriate deactivation or a gene necessary for cell survival or the subject's ability to thrive and/or survive. To treat this type of disease, an inhibitor of the RdRP activity of the composition of the invention, including either a TERT-RNA or TERT-RMRP complex, or a TERT polypeptide is administered to a subject in need thereof. As used herein, “inappropriate deactivation” is meant to describe the deletion, silencing, inaccessibility, methylation, mutation, or decreased gene dosage of a gene. In one aspect, this method is used to increase the effectiveness or abundance of a gene product if one copy of a gene is deleted or mutated, leaving a functional copy that might otherwise be regulated by gene silencing to control gene dosage. In this way, the remaining functional copy may compensate for the damaged copy. In another aspect, this method is used to reverse gene silencing in order to access functional copies of genes on silenced X-chromosomes when mutations or deletions have occurred on the non-silenced X-chromosome that cause deleterious consequences for the subject. In another aspect, this method is used to reverse or inhibit the inappropriate silencing of genes that should be active, for example at a particular time in development. In an additional aspect, this method is used to activate the expression or activity of genes that have redundant functions with genes that are deleted or mutated, as a compensatory mechanism. Finally, this method is used to reactivate or derepress genes in stem cells that prolong the ability of stem cells to remain undifferentiated as a way of promoting healing and cell replacement.

The invention provides a method of identifying an RNA molecule that forms a complex with a TERT polypeptide such that the resulting complex has RdRP activity. The method includes the steps of contacting the TERT polypeptide with a test RNA molecule to form a complex and identifying a complex that has RdRP activity. As used herein, the term “contacting” is meant to describe a process by which two molecules physically touch or come into physical proximity, e.g. both molecules are present in the same liquid. As used herein, the term “complex” is meant to describe the functional association of two molecules that may or may not have a physical association. In one aspect of the invention, the two molecules, for instance the TERT polypeptide and the RNA molecule, are physically bound by covalent or non-covalent bonds, e.g. electrostatic, hydrogen, van der Waals, π aromatic, and hydrophobic bonds. In another aspect of the invention, the two molecules, for instance the TERT polypeptide and the RNA molecule, are not physically bound to each other, but are associated with a common scaffold polypeptide, cytoskeletal element, lipid moiety, or polynucleic acid. As used herein, the term “RdRP activity” is defined as the ability to make an RNA copy of an RNA template. As such, a TERT-RNA complex has RdRP activity if a complementary strand of a single-stranded RNA template is synthesized in the presence of the TERT-RNA complex.

### Kits

The invention also includes a catalytic subunit (TERT) polypeptide and a means for detecting RNA polymerase (RdRP) activity packaged together in the form of a kit. Instructions (e.g., written, tape, VCR, CD-ROM, etc.) for carrying out the assay may be included in the kit. The assay may for example be in the form known in the art.

### EXAMPLES

### Example 1

### General Methods

### Cell Culture and Stable Expression of TAP-hTERT

The human cell lines 293T, MCF7, HeLa, HeLa—S and VA-13 were maintained in DMEM supplemented with 10% heat-inactivated fetal bovine serum (IFS). BJ fibroblasts were cultured as described (Hahn W. C. et al. Nature 400, 464 (1999)). Amphotropic retroviruses were created as described (2, 3) using the vectors pWZL-Blast-N-FLAGIHA-hTERT (for HeLa—S-TAP-hTERT), pBABE-puro or pBABE-puro-hTERT. After infection, cells were selected with blastcidin S (10 μg/ml) for 5 d or with puromycin (2 μg/ml) for 3 d.

Purification of hTERT Complexes and Cloning of RNAs

2×10⁸ HeLa—S cells expressing or lacking (control) TAP-hTERT were lysed in 5 ml of lysis buffer A (LBA; 20 mM Tris-HCl pH7.4, 150 mM NaCl, 0.5% NP-40, 0.1 mM DTT) and incubated for 30 min on ice. The lysate was then pelleted by centrifugation (16,000×g) for 20 min at 4° C. The supernatant was incubated with the anti-FLAG (M2) antibody conjugated agarose overnight at 4° C. The beads were washed 3 times with lysis buffer A and eluted with 3×FLAG peptide (150 ng/μl). The resulting elution was incubated with Protein A Sepharose beads and an anti-HA antibody (F7; Santa Cruz) for 4 h at 4° C. The beads were washed 3 times with lysis buffer A, and RNA was isolated using TRIzol (Invitrogen). RNA. samples prepared in this manner were analyzed using an Experion capillary electrophoresis device (Bio-Rad Laboratories, Inc. CA, USA) to visualize RNA species. For RNA cloning and the sequencing, the same samples were separated using a 7 M urea/15% acrylamide gel, and RNAs recovered from gel were cloned using the small RNA cloning Kit (TaKaRa).

### RNA Preparation for IP-RT-PCR

RNA samples that were prepared from the HeLa—S cells expressing TAP-hTERT as described above were also subjected to RT-PCR. For immunoprecipitation (IP) of endogenous hTERT complexes, cells (1×10⁸) were lysed in 600 μl of LBA, sonicated, and pre-cleared with 15 μl of 50% slurry of Protein A Sepharose (PAS, Pierce) for 2 h at 4° C. The pre-cleared total cell lysate was incubated with a rabbit polyclonal anti-hTERT antibody (Rockland, 2 μl) for 3 h at 4° C. followed by incubation with 30 μl of 50% slurry of PAS overnight at 4° C. After binding, the beads were washed 3 times for 30 min with LBA. RNA was isolated from the PAS using TRIzol (Invitrogen) followed by RT-PCR with primers specific for hTERC, RAMP or RNase P.

### RT-PCR

Either total cellular RNA or RNA from IP was isolated using TRIzol (Invitrogen) and subjected to RT-PCR. The following primers were used: hTERC (43F: 5′-TCTAACCCTAACTGAGAAGGGCGT-3′ (SEQ ID NO: 6) and 163R: 5′-TGCTCTAGAATGAACGGTGGAAGG-3 (SEQ ID NO: 7)) RMRP (F5: 5′-TGCTGAAGGCCTGTATCCT-3′ (SEQ ID NO: 8) and R257: 5′-TGAGAATGAGCCCCGTGT-3′ (SEQ ID NO: 9)), RNase P (F50: 5′-GTCACTCCACTCCCATGTCC-3′ (SEQ ID NO: 10) and R318: 5′-AATTGGGTTATGAGGTCCC-3′ (SEQ ID NO: 11)), and human β-actin (5′-CAAGAGATGGCCACGGCTGCT-3′ (SEQ ID NO: 12) and 5-TCCTTCTGCATCCTGTCGGCA-3′ (SEQ ID NO: 13)). The RT reaction was performed for 60 min at 42° C. using the recovered RNA, and PCR was immediately performed (21 cycles for 293T cells and 25 cycles for HeLa cells: 94° C., 30 s; 60° C., 30 s; 72° C., 30 s). To detect alphoid mRNA, following primers were used: (alphoid 29-F: 5′-GATGTGTGCGTT-3 (SEQ ID NO: 14) and alphoid 7-R: 5′-AGTTTCTGAGAATCATTCTGTCTAG-3′ (SEQ ID NO: 15) and PCR was performed (35 cycles: 94° C., 30 s; 60° C., 30 s; 72° C., 30 s).

### Quantitative RT-PCR

Quantitative RT-PCR was performed with a LightCycler 480 II (Roche) according to the manufacturer's protocols. The expression levels of RMRP was detected using the following primers and probe; forward primer (5′-GAGAGTGCCACGTGCATACG-3′ (SEQ ID NO: 36)), reverse primer (5′-CTCAGCGGGATACGCTTCTT-3′ (SEQ ID NO: 37)), VIC-labeled TaqMan MGB probe (5′-ACGTAGACATTCCCC-3′ (SEQ ID NO: 38)). β-actin was used as a reference.

Telomerase activity reconstituted in vitro and TRAP assay

In vitro reconstitution of telomerase activity (telomere specific reverse transcriptase activity) was performed as previously described (4). Briefly, recombinant hTERT was expressed in the TnT T7-Coupled Reticulocyte Lysate System (Promega) using the manufacturer's instructions. Purified hTERC or RMRP were included in the in vitro transcription/translation reactions. The telomeric repeat amplification protocol (TRAP) (1, 2, 5) was used to detect telomere specific reverse transcriptase activity.

### Affinity Purification of Recombinant GST-hTERT Fusion Proteins

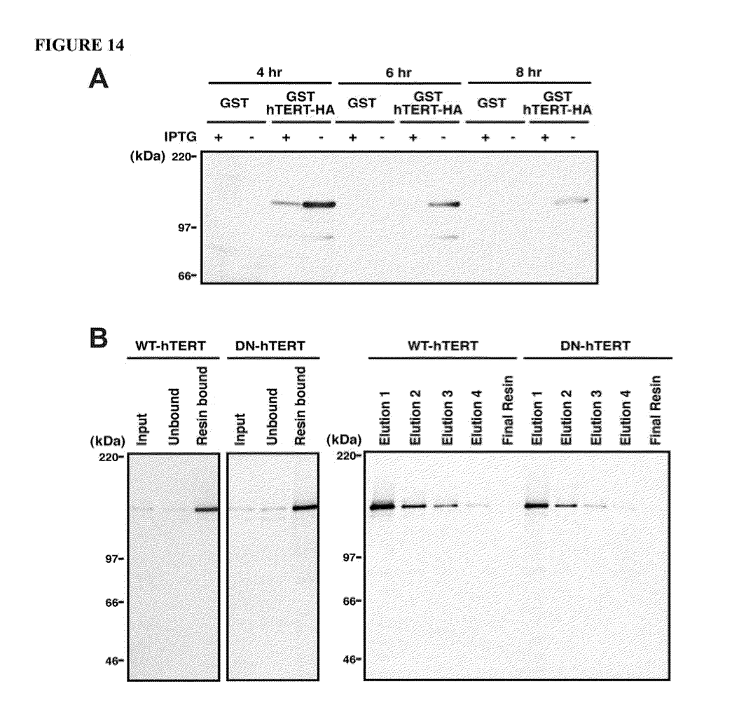

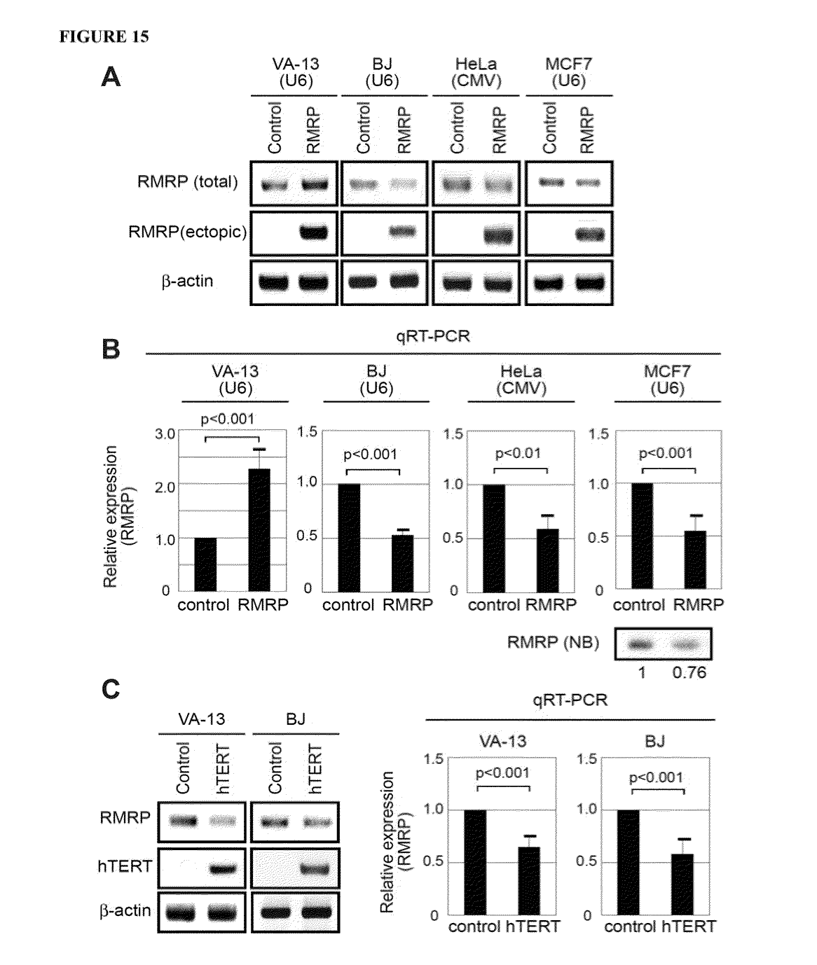

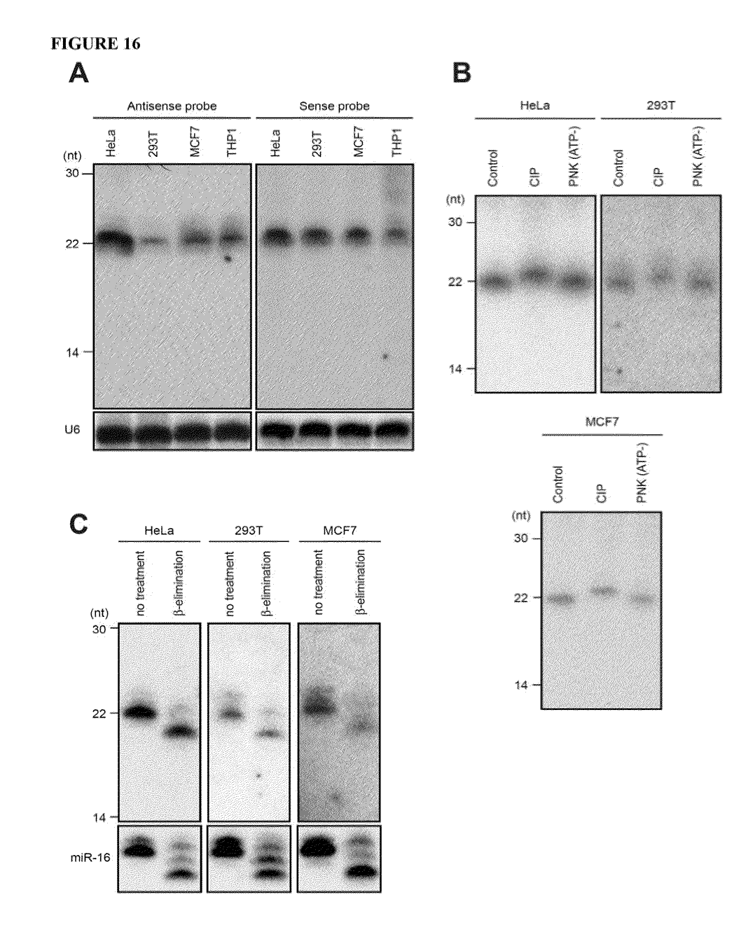

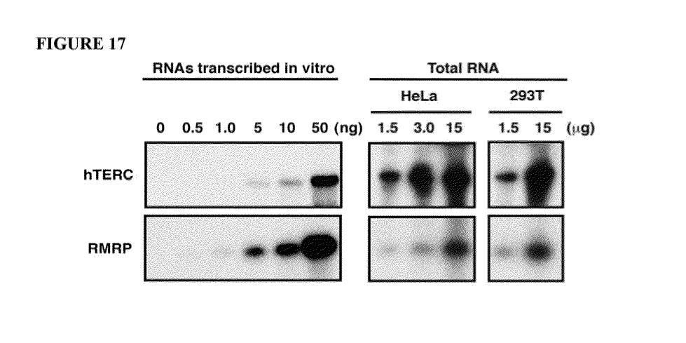

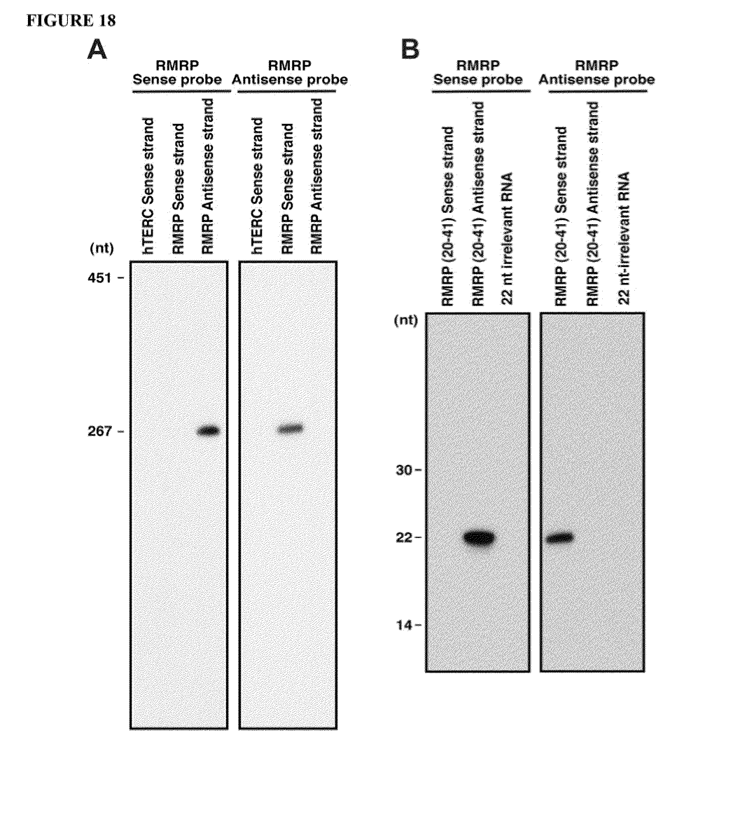

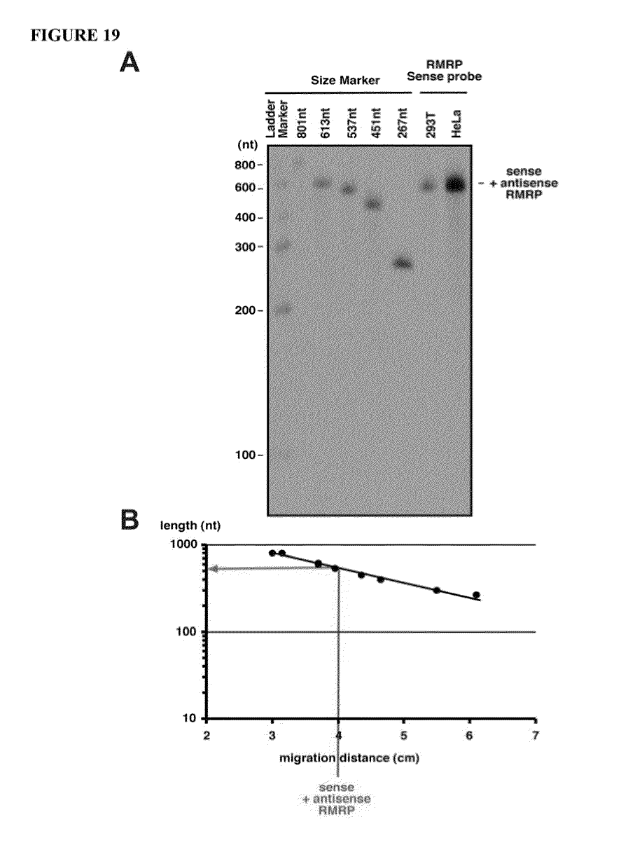

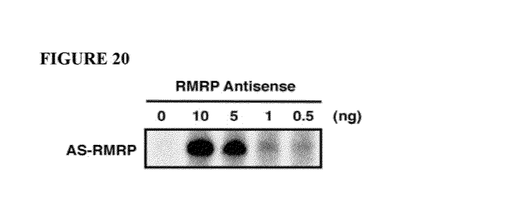

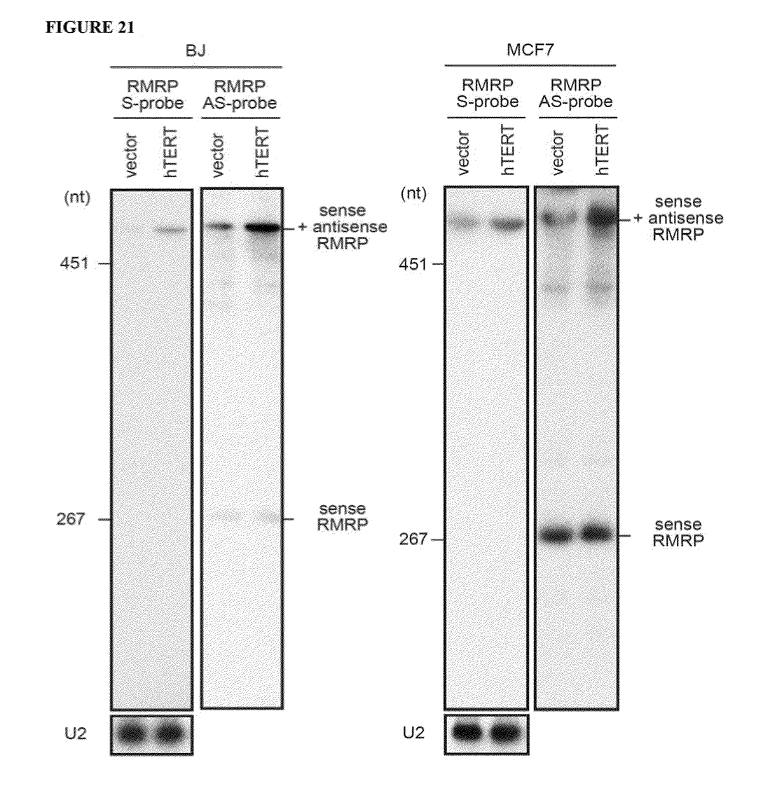

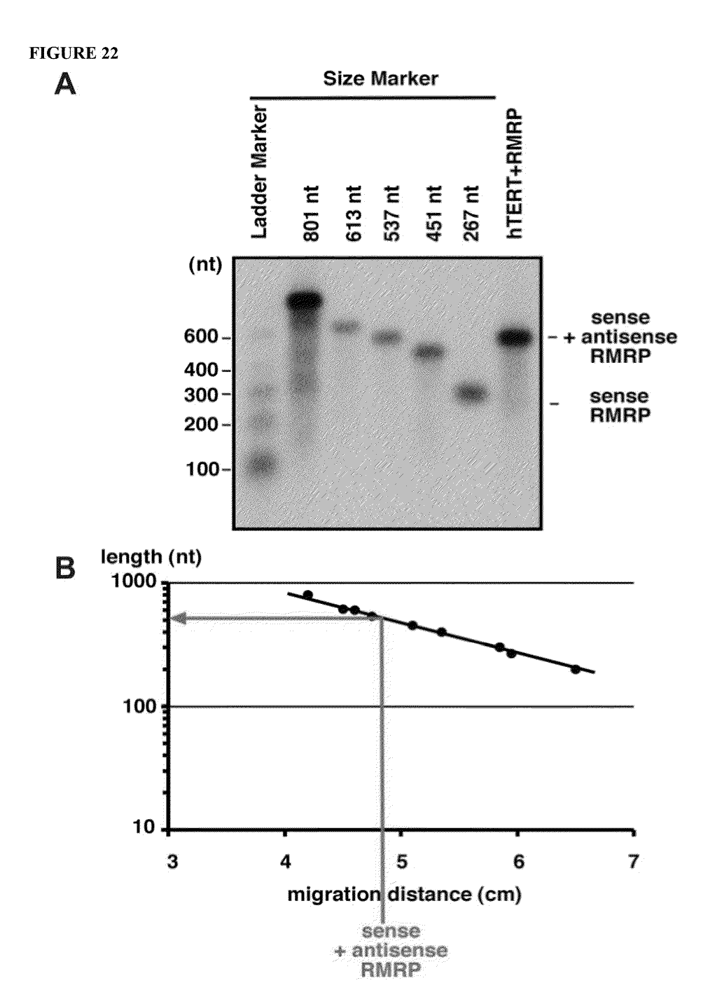

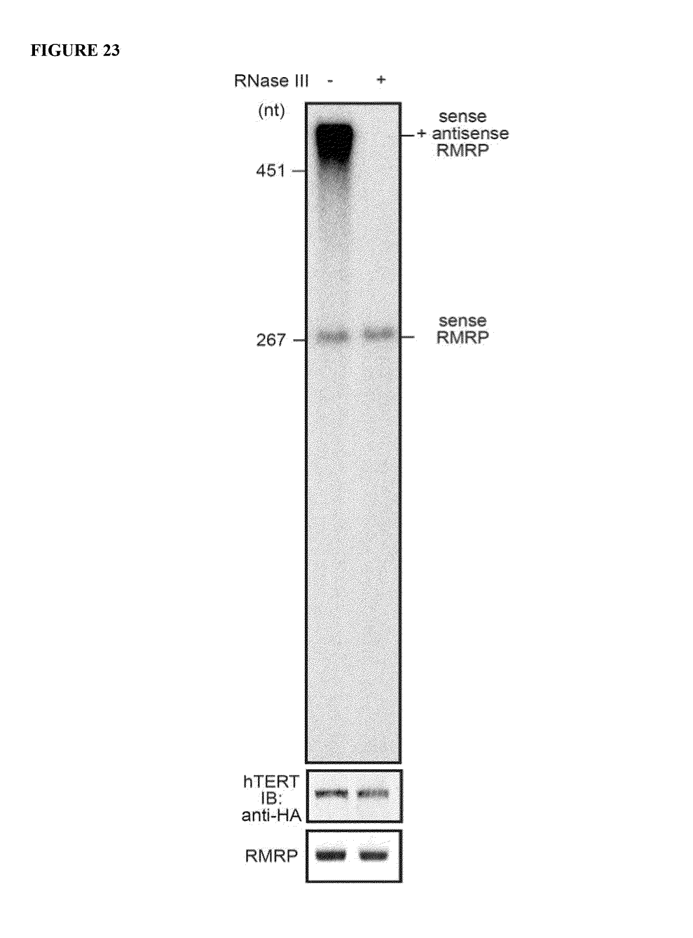

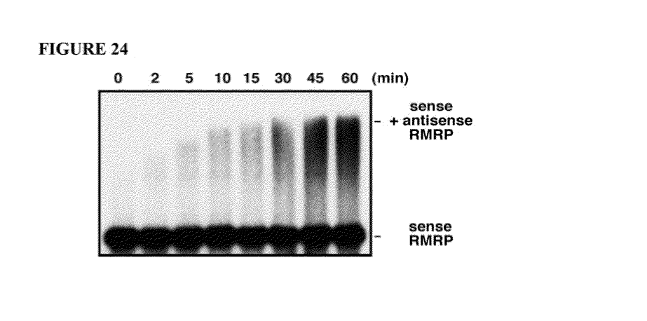

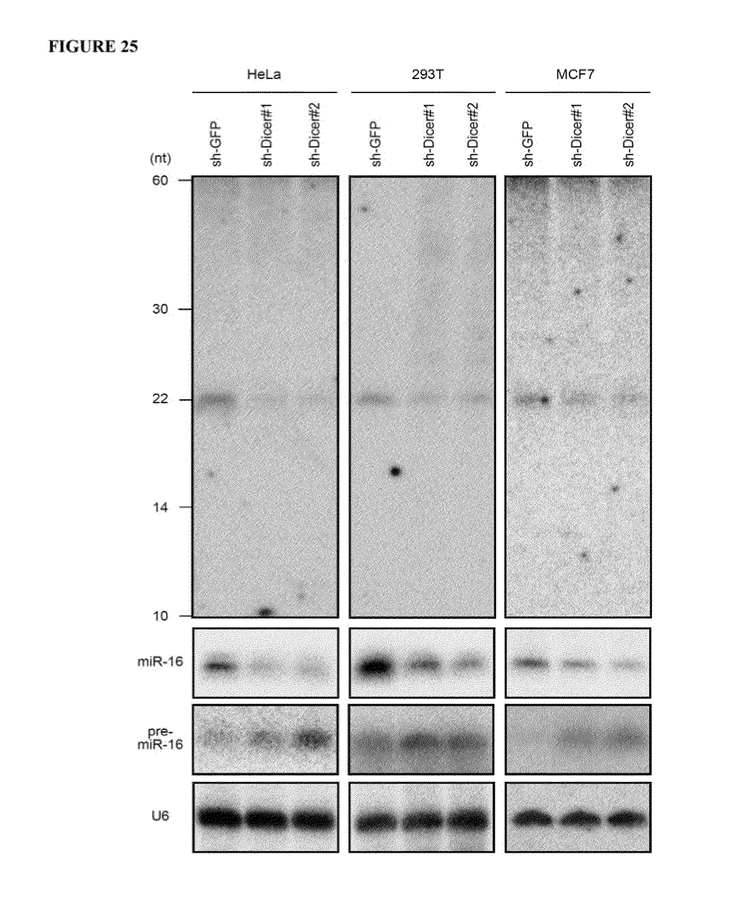

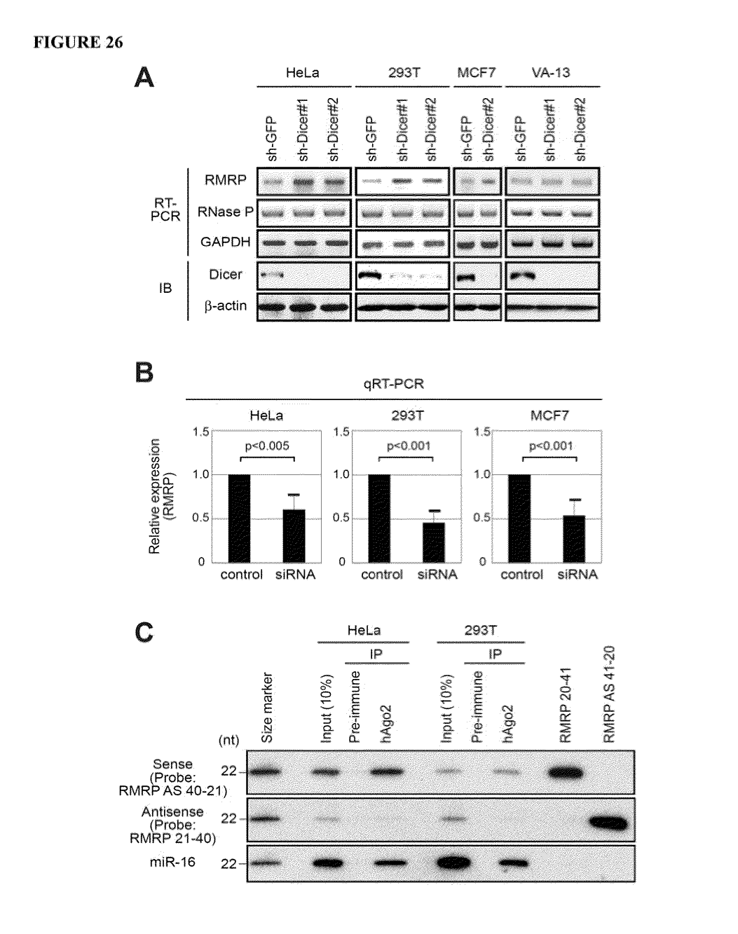

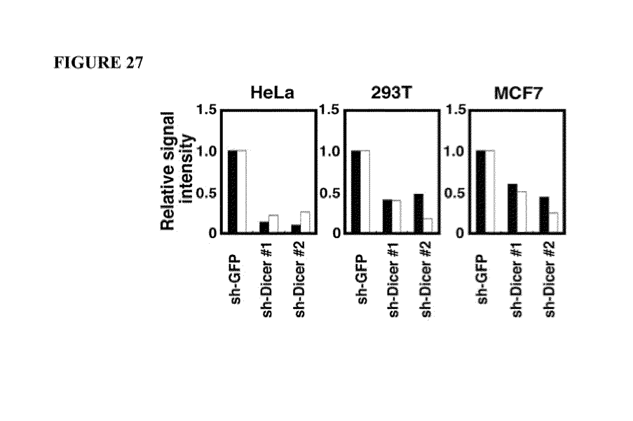

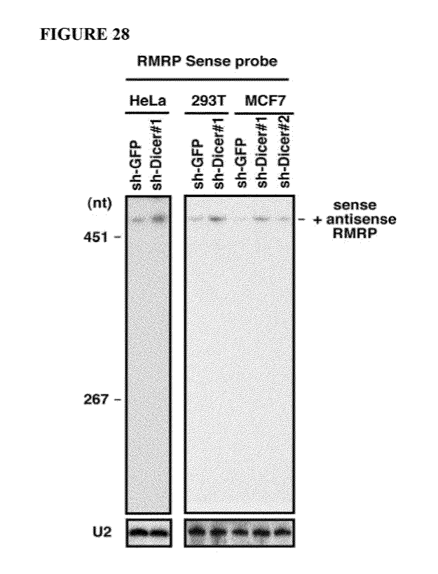

GST-hTERT-HA, GST-HT1 and GST-DN-hTERT proteins were expressed in BL21 bacterial cells (GST expression vector (pGENKZ) (6) was provided by Dr. Murakami (Cancer Research Institute, Kanazawa University) and incubated at 30° C. overnight. Thereafter 5 μl of this culture was re-inoculated into 5 ml of LB medium, incubated at 37° C. for 4 h, harvested by centrifugation, suspended in a lysis buffer [20 mM Tris-HCl pH7.4, 150 mM NaCl, 0.5% NP-40, 0.1 mM DTT, 10 mM PMSF, proteinase inhibitor (nacalai tesque)] and sonicated for 10 s at 4° C. After centrifugation of the sonicated lysates, the supernatants were passed through DEAE-Sepharose, and the GST fusion proteins were recovered using glutathione-Sepharose 4B beads. The resin was washed, and the GST fusion proteins were lien eluted with glutathione at 4° C. for 1 h [20 mM glutathione (reduced form)] in elution buffer [50 μM Tris-HCl pH8.8, 150 mM NaCl, 0.5% NP-40, 0.1 mMDTT, 10 mM PMSF, proteinase inhibitor (nacalai tesque)]. FIG. 14 shows that WT and DN hTERT were produced at similar levels using this method and the effects of incubation time and IPTG on yield. The average yield for this method is 500 ng (5 ng/μl) of active form of hTERT from 100 ml culture.

### RdRP Assay

10 ng of the affinity purified recombinant GST-hTERT fusion protein was incubated with 1 μg of RMRP-RNA transcribed in vitro in 200 mM KCl, 50 mM Tris-HCl (pH 8.3), 10 mM DTT, 30 mM MgCl₂, 50 μM rATP, 50 μM rGTP, 50 μM rCTP and 2 μCi of α-³²P-UTP at 32° C. for 2 h. Under low salt conditions, 20 μl of 0.2×SSC was then added to adjust final salt concentration to 15 mM NaCl and 1.5 mM sodium citrate, while under high salt condition 20 μl of 4×SSC was added to adjust final salt concentration to 300 mM NaCl and 30 mM sodium citrate. These mixtures were incubated at 37° C. for additional 1 h. Resulting products were treated with proteinase K to stop the reaction and purified with phenol/chloroform. To ensure that RNA products were completely denatured, we performed both conventional formamide treatment (with 95% formamide/20 mM EDTA gel loading buffer at 95° C. for 5 m.) and a further treatment with 1 M of de-ionized glyoxal at 65° C. for 15 m. To analyze double-stranded RNA produced by the hTERT-RMRP complex, we performed this RdRP assay and treated the products with RNase III (E. coli, Ambion, 50 mM NaCl, 10 mM Tris-HCl (pH 7.9), 1 mM DTT, 10 mM MgCl₂,) or RNase T1 (Roche, 50 mM Tris-HCl (pH 8.3), 300 mM NaCl and 30 mM sodium citrate).

### Northern Blotting

Total RNA and small RNAs (<200 nucleotides in length) were isolated using the mirVana miRNA Isolation Kit (Ambion) according to the manufacturer's protocol. 10 μg of total RNA or small RNA was separated on denaturing polyacrylamide gels, then blotted onto Hybond-N+membranes (GE Healthcare) using Trans-Blot SD Semi-Dry Transfer Cell (BIO-RAD). Hybridization was performed in Church buffer (0.5 M NaI pH 7.2, 1 mM EDTA and 7% SDS) containing 1×10⁶ cpm/ml of ³²P-labeled each probe for 14 h. The membranes were washed in 2×SSC, and the signals were detected by autoradiography.

Identification of Short RNA Species Derived from RMRP

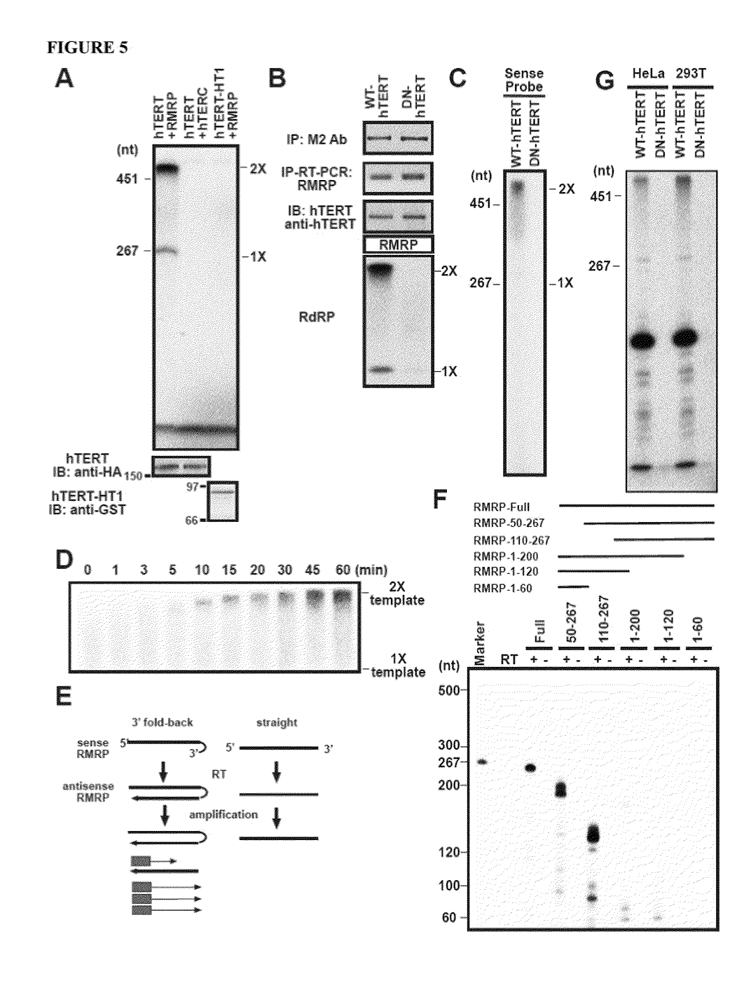

Using ten consecutive probes corresponding to the RMRP sequence, the small RNAs derived from RMRP shown in FIG. 5D were detected by a probe containing the complementary sequences to nucleotides 129-188 of RMRP. To determine the function of these RMRP-derived small RNAs, we designed two siRNAs targeting these 60 nt of RMRP using two different algorithms (Dharmacon and Invitrogen). Each of two synthesized siRNA (siRNA #1: 5′-gccaagaageguaucccgcuu-3′ (SEQ ID NO: 16) and siRNA #2: 5′-ccaagaagcguaucccgcuaa-3′ (SEQ ID NO: 17); Dharmacon) was transfected using Lipofectamine 2000 (Invitrogen) into 293T cells, HeLa cells and MCF7 cells plated on 6-well dishes according to the manufacturer's protocol. Using ten consecutive probes corresponding to the RMRP sequence, the small RNAs derived from RMRP shown in FIGS. 16A-C and FIG. 25 were detected by probes containing the complementary sequences to nucleotides 21-40 of RMRP. To determine the function of these RMRP-derived small RNAs, we purchased a chemically synthesized siRNA targeting this 20 nt portion of the RMRP sequence (siRNA: 5′-ggctacacactgaggactc-3′; Dharmacon) and transfected this siRNA into HeLa, 293T and MCF7 cells plated on 6-well dishes using Lipofectamine 2000 (Invitrogen) according to the manufacturer's protocol.

### RNase Protection Assay

RMRP RNA was transcribed with SP6 RNA polymerase in the presence of α-³²P-UTP using RiboMAX Large Scale RNA Production System (Promega). Total cellular RNAs (30 μg) were hybridized overnight at 60° C. with equal amounts of ³²P-labeled RMRP sense probe. Hybrids were digested with RNase A and RNase T1. The protected fragments were separated by PAGE under denaturing conditions and visualized by autoradiography.

### Analysis of the Chemical Structure of the Ends of Small RNAs

To determine the phosphorylation status of the termini of small RNAs, 30 μg of small RNA (<200 nucleotides in length) was treated with calf intestinal alkaline phosphatase (CIP; TaKaRa) for 2 h at 37° C. CIP was inactivated by phenol/chloroform extraction. Part of the CIP-treated RNA was then treated with T4 polynucleotide kinase (TaKaRa) supplemented with 1 mM ATP for 2 h at 37° C., and phenol/chloroform extraction was performed. 15 μg of small RNA was treated with T4 polynucleotide kinase without ATP for 2 h at 37° C. The reaction was inactivated by phenol/chloroform extraction. After overnight sodium acetate/ethanol precipitation at −20° C., the treated RNAs were resolved by 20% denaturing polyacrylamide/urea gel electrophoresis and then analyzed by Northern blotting. To further analyze the 3′ end of these small RNAs, we performed oxidation and β-elimination reactions. Specifically, the NaIO₄ reaction was performed by adding 20 μg of small RNAs in water to 5× borate buffer (148 mM borax and 148 mM boric acid, pH 8.6) and freshly dissolved 200 mM NaIO₄ to create a final concentration of 1× borate buffer and 25 mM NaIO₄. The mixtures were incubated for 10 min at 20° C. Glycerol was added to quench remaining NaIO₄, and the samples were incubated for an additional 10 min at 20° C. For β-elimination, small RNAs were dried by centrifugation and evaporation and dissolved in 50 μl of 1× borax buffer (30 mM borax, 30 mM boric acid and 50 mM NaOH, pH 9.5) and incubated at 45° C. for 90 min. Nucleic acids were recovered by sodium acetate/ethanol precipitation at −20° C. overnight, and the treated RNAs were resolved by 20% denaturing 7M urea PAGE and analyzed by Northern blotting.

### 3′ Primer Extension Assay

Truncated RMRP products inserted into pT7Blue2 vectors were transcribed using SP6 RNA polymerase (Promega). After intensive DNase I treatment, 100 ng of truncated RMRPs were reverse transcribed using Reverse Transcriptase M-MLV (RNase H—) (TaKaRa) without primers. Two microliters of these products were applied to amplifying steps with primers specific to newly synthesized ‘antisense’ cDNAs; RMRP-F5 for RMRP 1-267, RMRP 1-200, RMRP 1-120 and RMRP 1-60; RMRP-F50 (EcoRI) (5′-GCGAATTCCTCCCCTTTCCGCCTAG-3′ (SEQ ID NO: 18)) for RMRP 50-267; RMRP-F 110 (EcoRI) (5′-GCGAATTCGCACGTAGACATTCCCCG-3′ (SEQ ID NO: 19)) for RMRP 110-267. Each primer was end-labeled with γ-³²P-ATP using T4 Polynucleotide Kinase (TaKaRa). The 25 cycles of amplifying steps were performed in 25 μl of 1× buffer, containing 2 mM of MgCl₂; 0.2 mM each of dATP, dCTP, dGTP and dTTP; 0.625 U of TaKaRa Ex Taq (TaKaRa); and 0.2 μM of specific primers. Each cycle consisted of denaturation at 94° C. for 30 sec, annealing at 60° C. for 30 sec and extension at 72° C. for 30 sec. Amplified products were separated in 5% polyacrylamide gels containing 7M urea and visualized by autoradiography.

Stable Expression of shRNA

The pLKO.1-puro vector and the sequences described below were used to create shRNA vectors specific for hTERT, RMRP, Dicer and GFP. These vectors were used to make amphotropic retroviruses and polyclonal cell populations were purified with selection with puromycin (2 μg/ml). The sequences used for the indicated short hairpin RNAs are shown below where the capitalized letters represent the targeting sequences.

sh-hTERT#1:
(SEQ ID NO; 20)
5′GGAAGACAGTGGTGAACTTCCctcgagGGAAGTTCACCACTGTCTTCC
ttttt-3′
and
(SEQ ID NO: 21)
5′-aattcaaaaaGGAAGACAGTGGTGAACTTCCctcgagGGAAGTTCAC
CACTGTCTTCC-3′;
sh-hTERT#2:
(SEQ ID NO: 22)
5′-GGAACACCAAGAAGTTCATCTctcgagAGATGAACTTCTTGGTGTTC
Cttttt-3′
and
(SEQ ID NO: 23)
5′-aattcaaaaaGGAACACCAAGAAGTTCATCTctcgagAGATGAACTT
CTTGGTGTTCC-3′.
RMRP sequences.
sh-RMRP#1;
(SEQ ID NO: 24)
5′-GCAGAGAGTGCCACGTGCAttcaagagaTGCACGTGGCACTCTCTGC
tttttg-3′
and
(SEQ ID NO: 25)
5′-aattcaaaaaGCAGAGAGIGCCACGTGCAtctcttgaaTGCACGTGG
CACTCTCTGC-3′.
sh-RMRP#2;
(SEQ ID NO: 26)
5′-GCCTGTATCCTAGGCTACACActcgagTGTGTAGCCTAGGATACAGG
Ctttttg-3′
and
(SEQ ID NO: 27)
5′-aattcaaaaaGCCTGTATCCTAGGCTACACActcgagTGTGTAGCCT
AGGATACAGGC-3′.
Dicer sequences
sh-Dicer#1;
(SEQ ID NO: 28)
5′-GCTCGAAATCTTACGCAAATActcgagTATTTGCGTAAGATTTCGAG
Ctttttg-3′
and
(SEQ ID NO: 29)
5′-aattcaaaaaGCTCGAAATCTTACGCAAATActcgagTATTTGCGTA
AGATTTCGAGC-3′
sh-Dicer#2;
(SEQ ID NO: 30)
5′-CCACACATCTTCAAGACTTAActcgagTTAAGTCTTGAAGATGTGTG
Gtttttg-3′
and
(SEQ ID NO: 31)
5′-aattcaaaaaCCACACATCTTCAAGACTTAActcgagTTAAGTCTTG
AAGATGTGTGG-3′
sh-hTERC#1;
(SEQ ID NO: 32)
5′-TTGTCTAACCCTAACTGAGAActcgagTTCTCAGTTAGGGTTAGACA
Atttttg-3′
and
(SEQ ID NO: 33)
5′-aattcaaaaaTTGTCTAACCCTAACTGAGAActcgagTTCTCAGTTA
GGGTTAGACAA-3′;

sh-hTERC #2 provided by Elizabeth Blackburn (Li, S. et at Cancer Res 64, 4833 (2004)).

The control retroviral vector encoding a GFP-specific shRNA was created in pLKO.1-puro with the oligonucleotides

(SEQ ID NO: 34)
5′-CGCAAGCTGACCCTGAGTTCATTCAAGAGATGAACTTCAGGGTCAGC
TTGCTTTTTG-3′
and
(SEQ ID NO: 35)
5′-AATTCAAAAAGCAAGCTGACCCTGAAGTTCATCTCTTGAATGAACTT
CAGGGTCAGCTTGCGGGCC-3′.

### Immunoprecipitation of Human Ago2 Complexes

HeLa cell or 293T cell lysates were prepared with the lysis buffer A and immunoprecipitated by anti-hAgo2 antibodies (kindly provided by Dr. Haruhiko Siomi and Dr. Ivlikiko C. Siomi, KeioUniversity). RNA was isolated using TRIzol from the protein A beads and resolved by electrophoresis on 7M Urea 20% PAGE. Small RNAs were detected by Northern blotting with antisense probe, sense probes derived from nt 21-40 of RMRP, or miR-16 specific probe (5′-CGCCAATATTTACGTGCTGCTA-3′ (SEQ ID NO: 39)).

### Immunofluorescence (IF)

For IF, cells were fixed with 3.7% formaldehyde/2% sucrose, permeabilized by 0.5% Triton X-100, incubated with the indicated primary antibody [anti-trimethyl-Histone H3 (Lys9): Upstate (#07-442); anti-HP1-β: Upstate (#07-333); anti-acetyl-Histone H3: Upstate (#06-599): and anti-CENP-A clone 3-19: MBL] washed and then incubated with an AlexaFluor488-conjugated secondary antibody (Invitrogen) in 1% BSA for 1 h at 37° C. Cells were imaged with an IX81 inverted microscope with DSU (disc scan unit) (Olympus, Tokyo, Japan) and an ORCA-AG cooled CCD camera (Hamarnatsu Photonics K,K, Shizuoka, Japan). MetaMorph software was used for control of the CCD camera and filter wheels, and also to perform the statistical analysis of the cell image data.

Quantitative analysis of relative imsnunofluorescence intensity was performed using MetaMorph software. Briefly, for a specific primary antibody, 50 nuclei from each sample were randomly selected and outlined based on the DAPI signals. The fluorescent intensities of both Alexa 488 on secondary antibodies and DAPI were summed, respectively, on a per nucleus basis. Relative fluorescent intensity was calculated for each nucleus as the ratio of the total intensity of Alexa 488 to the intensity of DAPI as described previously (O'Sullivan, J. N. et al. Nat Genet. 32, 280(2002; McManus, K. J. and Hendzel, M. J. Mol. Cell Biol 23, 7611 (2003); Maida, Y. et al. J Pathol 210, 214 (2006); McManus, K. J. et al. J Biol Chem 281, 8888 (2006); Sakaue-Sawano, A. et al. Cell 132, 487 (2008)). p-values were obtained using a two-tailed t-test.

### Example 2

### Identification of a Second RNA that Interacts with hTERT

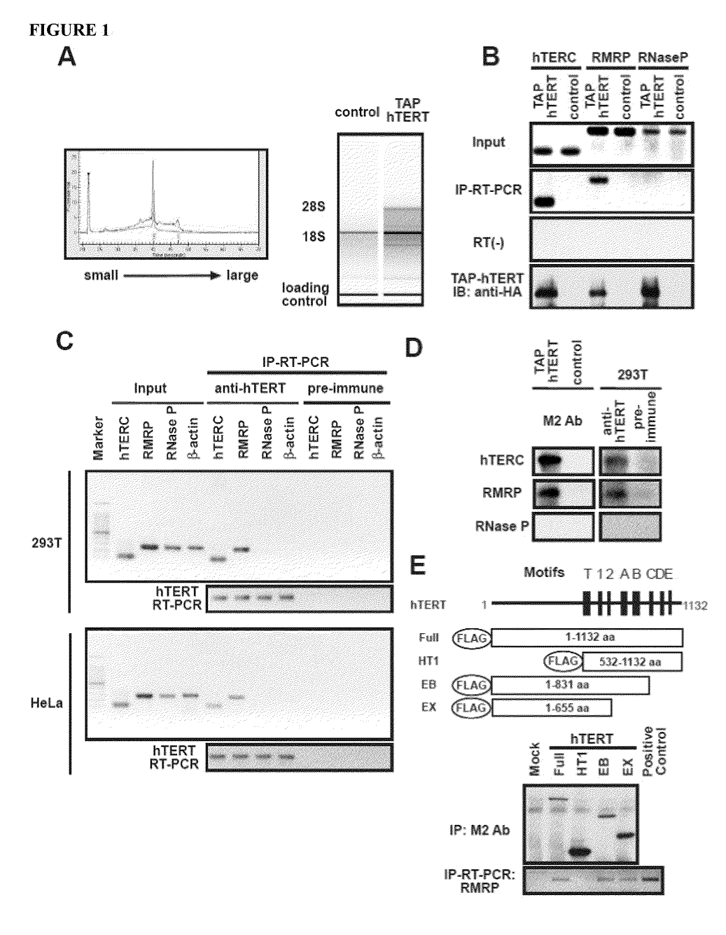

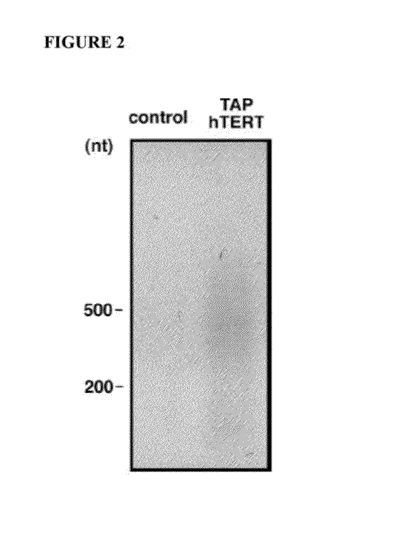

To identify additional hTERT partners involved in these telomere independent functions of hTERT, a tandem affinity purification (TAP)-tagged hTERT protein was stably overexpressed in HeLa—S cells and isolated hTERT immune complexes. Since some of the telomere independent functions of TERT do not require the presence of the TERC subunit (Sarin, K. Y, et al., Nature 436, 1048 (2005); Blackburn, E. H. Nature 436, 922 (2005); Lee, J. et al., Oncogene (2008)), RNA species associated with these TERT immune complexes were examined to identify other associated RNAs. A heterogeneous mixture of RNAs less than 1000 nt in length associated with TAP-hTERT was identified (FIGS. 1A and 2). After cloning and sequencing these RNAs, 38 sequences associated with the hTERT complex were identified. 5% (2/38) of the sequences corresponded to hTERC (Table 1). In addition to hTERC, it was determined that the same number of sequences matched the RNA component of mitochondrial RNA processing endoribonuclease (RMRP). RMRP was initially identified in mitochondria but is also a small nucleolar (sno) RNA like hTERC (Calado, R. T. and Young, N. S. Blood 111, 4446 (2008); M. Ridanpaa et al., Cell 104, 195 (2001)), and mutations of RMRP are found in the pleiotropic inherited syndrome, Cartilage-Hair Hypoplasia (CHH) (Tollervey, D. and Kiss, T. Curr Opin Cell Biol 9, 337 (1997).

It was confirmed that either overexpressed or endogenous hTERT interacts with RMRP by isolating TAP-hTERT (FIG. 1B) or endogenous hTERT (FIG. 1C) complexes in both HeLa and 293T cells under conditions in which RNase P was not recovered. Although other RNAs also co-purified with hTERT (Table 1), the interaction of Alu sequences or the 5.8S ribosomal RNA on chromosome Y with hTERT was not confirmed. Indeed, when RNA found in hTERT immune complexes was subjected to Northern blotting analysis, co-immunoprecipitation of hTERT with RMRP or hTERC was identified at similar abundance even though hTERC was expressed at approximately five- to ten-fold higher levels than RMRP in these cells (FIG. 1D and FIG. 17).

To further characterize the interaction of hTERT and RMRP, TERT truncation mutants were used and demonstrated that the aminoterminal end of hTERT (1-531) (HT1 mutant), a portion of hTERT unique to mammalian TERT, was necessary for hTERT to interact with RMRP (FIG. 1E). Two regions in the aminoterminal end of hTERT (amino acids 30-159 and 350-547) are necessary for the binding of hTERC (Calado, R. T. and Young, N. S. Blood 111, 4446 (2008); Moriarty, T. S. et al. Mol Cell Biol 22, 1253 (2002)). Taken together, these observations demonstrate that hTERT and RMRP form a novel ribonucleoprotein complex and that hTERT forms distinct complexes with RMRP and hTERC.

TABLE 1
hTERT associated RNAs.

| | Numbers of | Sequence |
|---|---|---|
| Matched sequence name | sequence | Identity (%) |
| hTERC | 2 | 100% |
| RMRP | 2 | 100% |
| Segment of chromosome 21 | 1 | 100% |
| Immunoglobulin mu heavy chain-like | 1 | 100% |
| Alu repeat sequences | 2 | 100% |
| mt-tRNA for glutamine | 1 | 100% |
| mt-tRNA for aspartate | 2 | 99% |
| mt-tRNA for arginine | 3 | 99% |
| mt-tRNA for valine | 15 | 99% |
| tur-tRNA for proline | 1 | 99% |
| int-IRNA for glycine | 1 | 99% |
| 5.8S ribosomal RNA on chromosome Y | 2 | 94% |
| mt-tRNA for cysteine | 1 | 92% |
| mt-tRNA for phenylalanine | 1 | 78% |
| mt-tRNA for lysine | 1 | 73% |
| mt-tRNA for tryptophan | 2 | 67% |

### Example 3

### The hTERT-RMRP Complex Exhibits RNA-Dependent RNA Polymerase (RdRP) Activity

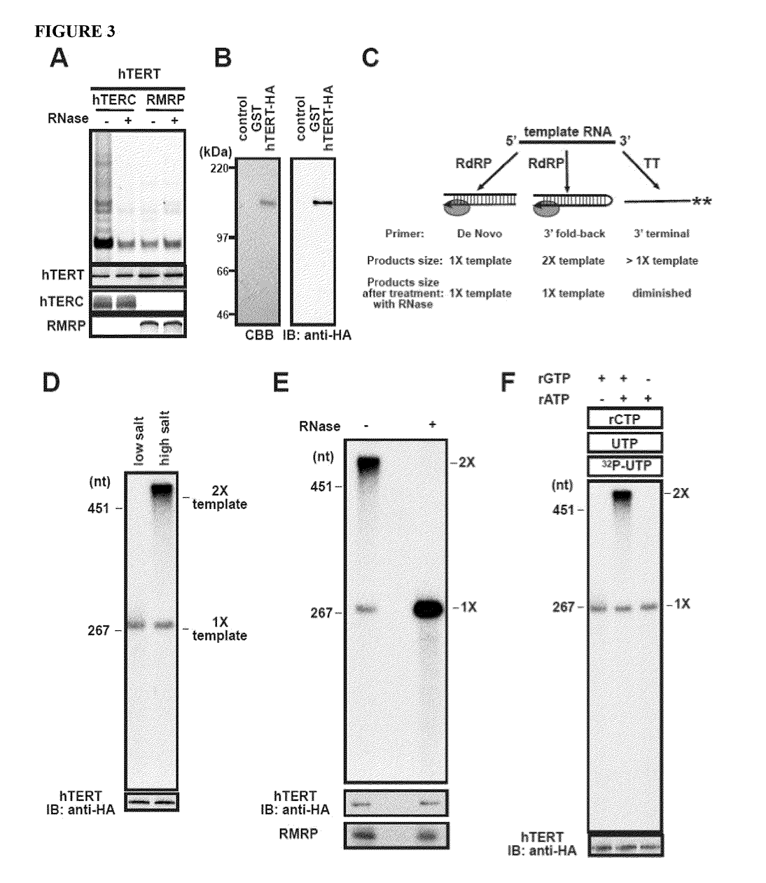

hTERT and hTERC form telomerase, a specialized RNA dependent DNA polymerase that synthesizes telomeric repeats. To test whether RMRP substitutes for hTERC to reconstitute telomere reverse transcriptase activity, recombinant hTERT produced in a rabbit reticulocyte system was combined with hTERC or RMRP RNAs transcribed in vitro. As expected, telomerase (telomere specific reverse transcriptase) activity was detected when hTERT and hTERC were combined (FIG. 3A). In contrast, telomerase activity was not detected when hTERT and RMRP were co-incubated, indicating that the complex composed of hTERT and RMRP does not exhibit telomerase activity (FIG. 3A).

In complex with hTERC, hTERT acts as a telomere specific reverse transcriptase, and TERT has been shown to act as a terminal transferase (Lue, N. F. of al., Proc Natl Acad Sci USA 102, 9778 (2005)). In addition, hTERT shares distant sequence similarity to a discrete subgroup of polymerases closely related to RNA dependent RNA polymerases (RdRP) found in positive-stranded RNA viruses such as poliovirus (Nakamura, T. M. et al., Science 277, 955 (1997)). RdRPs have recently been shown to participate in the endogenous RNA interference (RNAi) pathway and in the regulation of posttranscriptional gene silencing (PTGS) in plants and other eukaryotes (Mourrain, P. et al., Cell 101, 533 (2000); Nishikura, K. Cell 107, 415 (2001); Makeyev, E. V. and Bamford, D. H. Mol Cell 10, 1417 (2002); Du, T. and Zamore, P. D. Development 132, 4645 (2005); Almeida, R. and Allshire, R. C. Trends Cell Biol 15, 251 (2005). To examine whether the complex formed by hTERT and RMRP exhibits RdRP and/or terminal transferase activity, an RNA synthesis activity assay was established with recombinant, affinity-purified hTERT protein (FIG. 3B) and RNA molecules transcribed in vitro. In this assay, three modes that the hTERT-RMRP complex might use to elongate RNA were predicted. Specifically, the hTERT-RMRP complex could act [i] as an RdRP using a de novo synthesized RNA primer to elongate a complementary strand (FIG. 3C left panel), [id] as an RdRP that uses a 3′ fold-back (back-priming) configuration of RMRP as a primer (FIG. 3C middle panel) or [iii] as a terminal transferase (FIG. 3C right panel). Viral RdRPs, such as those found in poliovirus (B. L. Semler, E. Wimmer, Molecular Biology of Picornaviruses (AMS Press, Washington, D.C., 2002), pp. 255-67)., hepatitis C virus (Behrens, S. E. et al. EMBQ J 15, 12 (1996)), Dengue virus (Ackermann, M. and Padmanabhan, R. J Biol Chem 276, 39926 (2001)) and influenza virus (Engelhardt, O. G. and Fodor, E. Rev Med Virol 16, 329 (2006)), have been shown to use either of the first two modes to prime RdRP activity. Moreover, the RdRP in fission yeast (Sugiyama, T. et al. Proc Natl Acad Sci USA 102, 152 (2005)) and fungi (Makeyev, E. V. and Bamford, D. H. Mol Cell 10, 1417 2002)) use similar priming mechanisms to produce double stranded RNAs that serve as precursors for RNAi.

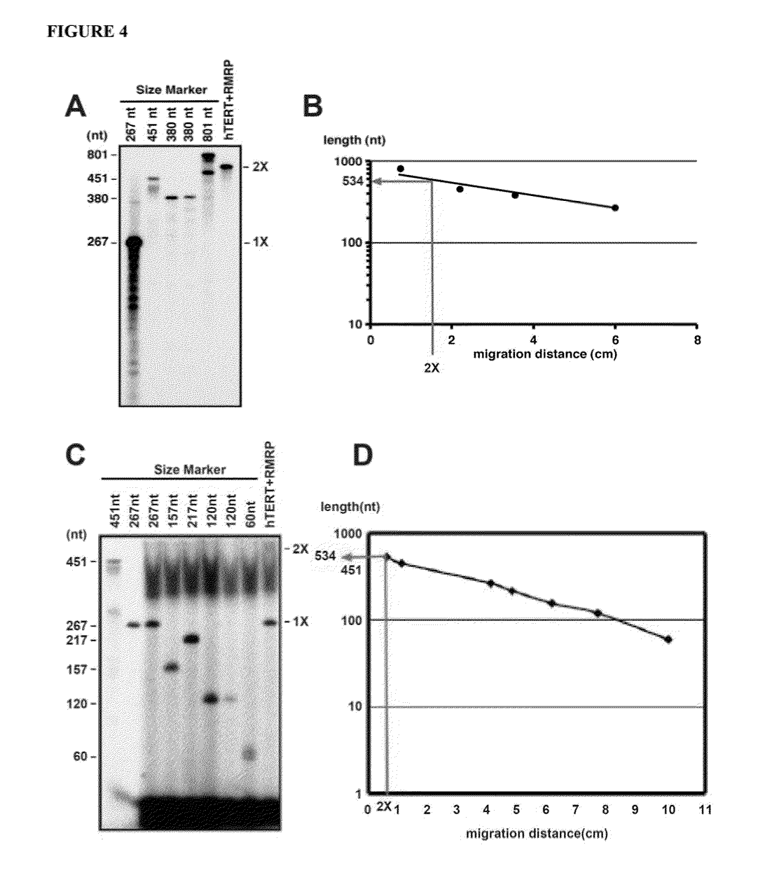

It was determined that the complex of hTERT and RMRP produced 3 different products depending on the salt concentration in the presence of Mg²⁺ (FIG. 3D). Specifically, we found 1× (267 nt) and 2× template (534 nt) sized products under high salt conditions (300 mM NaCl and 30 mM sodium citrate) and a slightly longer than 1× template sized product under low salt conditions (15 mM NaCl and 1.5 mM sodium citrate). The size of these products was confirmed by co-electrophoresis with RNAs of known length (FIGS. 4A-D and FIGS. 22A, B). To discriminate among these three different modes, the products were treated (FIG. 3E) with RNase T1, which digests single stranded RNA, after performing an RdRP assay in vitro. RNase treatment completely eliminated the slightly longer than 1× template length RNA products produced under low salt concentrations, indicating that ³²P-UTP was incorporated by terminal transferase activity.

In contrast, when the assay was performed under high salt conditions, two RNAs (1× template sized and 2× template sized products) were found that collapsed into a single RNA product (1× template size) after treatment with RNase T1 (FIG. 3E). To eliminate the possibility that the 2× template sized product represented partially denatured RNAs, we also treated the products of the RdRP assay with bacterial RNase III, which digests dsRNA, and found that only the input ssRNA remained (FIG. 23). Furthermore, when this in vitro RdRP assay was performed under conditions where one of the four ribonucleotides (adenine or guanine ribonucleotides) was left out, the 2× template sized products could not be detected (FIG. 3F). These observations confirm that the 2× template sized products are formed by RdRP activity and represent a double-stranded hairpin structure created by a single RNA molecule composed of the sense and antisense strand of RMRP.

To confirm that the interaction of hTERT and RMRP was required for the observed RdRP activity, an RdRP activity assay was performed using combinations of recombinant hTERT proteins and RMRP RNA transcribed in vitro. As expected, the RdRP reaction products were not detected when hTERT and hTERC were co-incubated. Moreover, when the hTERT-HT1 mutant was used, which does not bind RMRP (FIG. 1E), labeled RNA products were not observed (FIG. 5A) under conditions where two different RNA products in reactions containing wild type hTERT and RMRP were detected. An hTERT mutant (DN hTERT) that harbors a mutation in a conserved residue in the catalytic domain and that fails to elongate telomeres when expressed in human cells has been described (Masutomi, K. et al., Proc Natl Acad Sci USA 102, 8222 (2005); Masutomi, K. et al., Cell 114, 241 (2003)). It was confirmed that this DN hTERT mutant retains the ability to bind RMRP (FIG. 5B). However, the DN hTERT-RMRP complex lacks detectable RdRP activity (FIG. 5B). Thus, hTERT serves as the catalytic subunit for both the telomerase reverse transcriptase and RdRP activities.

### Example 4

### The hTERT-RMRP RdRP Produces Double-Stranded RNA (dsRNA)

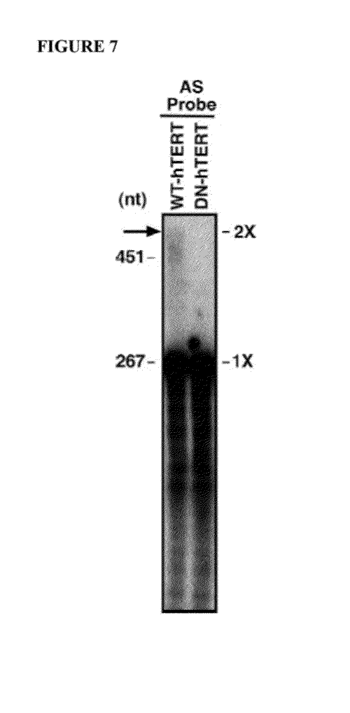

The hTERT-RMRP RdRP synthesizes double-stranded RNA in a template dependent manner. To confirm that the synthesis of the complementary strand of RMRP could be detected in the in vitro RdRP assay, the sense strand of RMRP was used as a probe to perform a Northern blot analysis of products from this assay. As expected, the antisense strand of RMRP was detected in reactions containing recombinant WT hTERT protein and RMRP transcribed in vitro (FIG. 5C left lane). Reactions that contained recombinant DN hTERT and RMRP transcribed in vitro failed to produce the complementary strand of RMRP (FIG. 5C right lane). Furthermore, in a Northern blot analysis, a 2× template sized product was detected in the in vitro RdRP assay using the antisense strand of RMRP as a probe (FIG. 7). These results indicate that the RdRP formed by hTERT in combination with RMRP produces double-stranded RNAs in template dependent manner in vitro.

Although the production of both 2× and 1× template sized RMRP was observed in vitro, the 2× template sized RNA products were reproducibly more abundant than the 1× template sized RMRP (FIGS. 3D and 5A). These results indicate that the hTERT-RMRP RdRP favors a back priming mechanism for the priming process in these cell lines. To test this model, the priming process was examined using hTERT and RMRP as a model system. RdRP activity was monitored over time and it was found that 2× template sized RMRP products appeared in a time dependent manner (FIG. 5D). When this assay was performed using radiolabeled RMRP as a substrate, we found that a portion of the labeled RMRP was similarly extended (FIG. 24). These experiments further confirm that hTERT-RMRP can use RMRP as a primer and template.

To determine whether the RMRP RNA forms a fold-back configuration at the 3′ end and to determine the portion of RMRP necessary for this mode of priming, several RMRP truncation mutants were generated and a 3′ primer extension assay was established (FIGS. 5E and F). Using these experimental conditions, a steady state level of DNA products from the RMRP-RNA mutants that contain intact 3′ regions was detected. In contrast, when RMRP truncation mutants that lack the 3′ end were used in this assay, no RMRP-derived products could be detected (FIG. 5F). Thus, unlike what has been described in fission yeast, the hTERT-RMRP exhibits a restricted preference for RNA molecules that can be used as a template. Indeed, when purified recombinant hTERT was incubated together with total cell RNA and ³²P-UTP, a limited number of labeled RNAs were identified (FIG. 5G). Although RMRP has been represented as a linear molecule, it is recognized that RMRP may form a more complex secondary structure in vivo to create the 3′ fold-back necessary for complementary strand synthesis. Nevertheless, these results indicate that RMRP can itself serve as a primer for the polymerization process using fold-back formation at the 3′ end and that hTERT can elongate the complementary strand through RdRP activity.

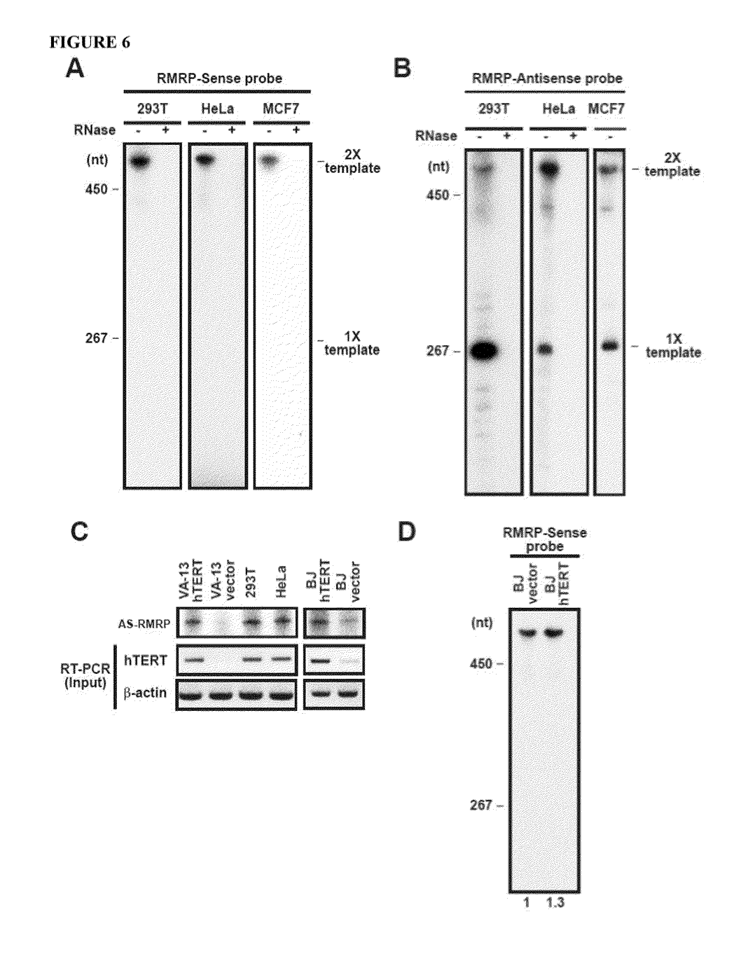

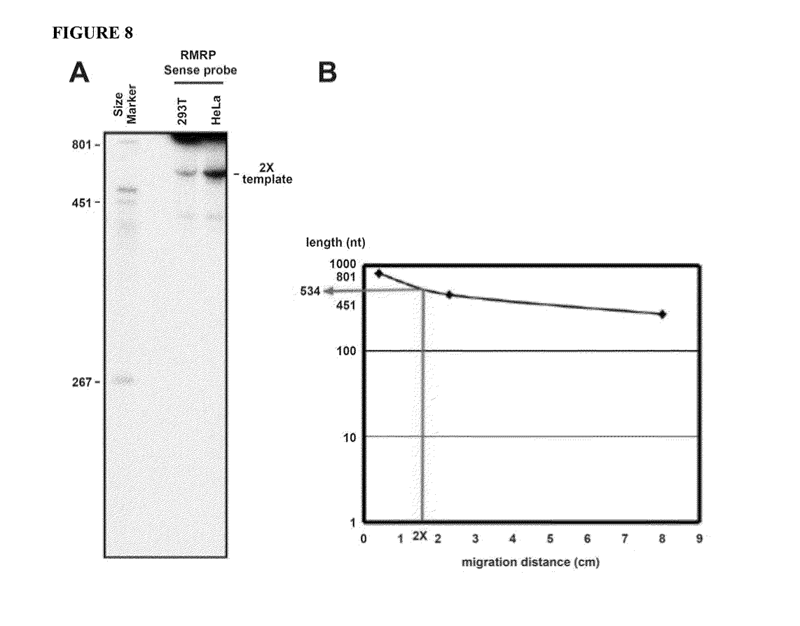

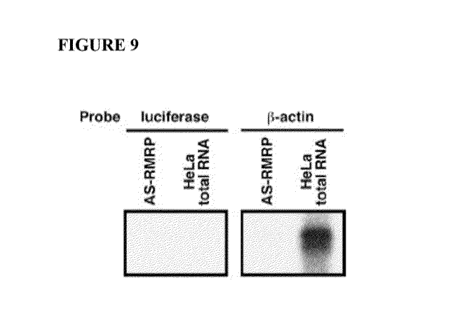

To determine whether synthesis of the antisense strand of RMRP also occurs in vivo, the sense and antisense strand probe of RMRP was used to detect sense and antisense RMRP in total RNA isolated from human cell lines. The specificity of the probes was confirmed (FIG. 18A). Discrete 2× template sized antisense RMRP were detected in 293T cells, HeLa cells and MCF7 cells using a sense strand probe (FIGS. 6A, 8A and 8B, and FIG. 19). Moreover, 2× template sized products as well as 1× template sized products were detected using antisense strand probe of RMRP (FIGS. 6B, 5A and 8B). These results confirmed that that the 2× template sized products contain the RMRP sense strand and antisense strand. To determine whether the expression of hTERT is necessary for the appearance of RMRP in cells, the levels of the complementary RMRP strand in three classes of cells were examined: (i) Cells that do not express hTERT and hTERC (VA-13) (Ford, L. P. et al., J Biol Chem 276, 32198 (2001)); (ii) Cells that transiently express low levels of hTERT and constitutively express hTERC (3J) cells (Masutomi, K. et al., Cell 114, 241 (2003)); and (iii) Cells that constitutively express hTERT and hTERC (293T and HeLa). For the VA-13 and BJ cells, a control expression vector or an expression vector that encodes hTERT was also introduced. RNA from these cells was isolated and 2× template sized RMRP products was detected using both a quantitative RNase protection assay with a sense strand-specific probe that detects both forms of RMRP (2× and 1× template sized) as a single species (FIGS. 6C and 9 and FIG. 20) and a Northern blot analysis with a sense strand-specific RMRP probe and an anti sense strand-specific RMRP probe (FIG. 6D and FIG. 21). It was discovered that the expression levels of 2× template sized RMRP products correlated with the expression of hTERT (FIGS. 6C, D and FIG. 21). Taken together, these results confirmed that the TERT-RMRP RdRP produces double-stranded RMRP in vivo.

### Example 5

### Effects of the hTERT and RMRP Complex on siRNA and PTGS

In many organisms. RdRPs play a central role in the synthesis of double-stranded RNA that are processed into siRNA to mediate PTGS. Because the RdRP formed by hTERT and RMRP produces double stranded RNA, it was hypothesized that the hTERT-RMRP complex produces RMRP-specific siRNA to regulate RMRP RNA expression levels. To assess the consequences of overexpressing the hTERT-RMRP complex on RMRP levels, retroviral vectors were used to introduce RMRP into cells lacking hTERT expression (VA-13), cells that transiently express hTERT in a cell-cycle dependent manner (BJ fibroblasts) and cells that constitutively express hTERT (VA-13) expressing ectopic hTERT, BJ fibroblasts expressing ectopic hTERT and HeLa cells).

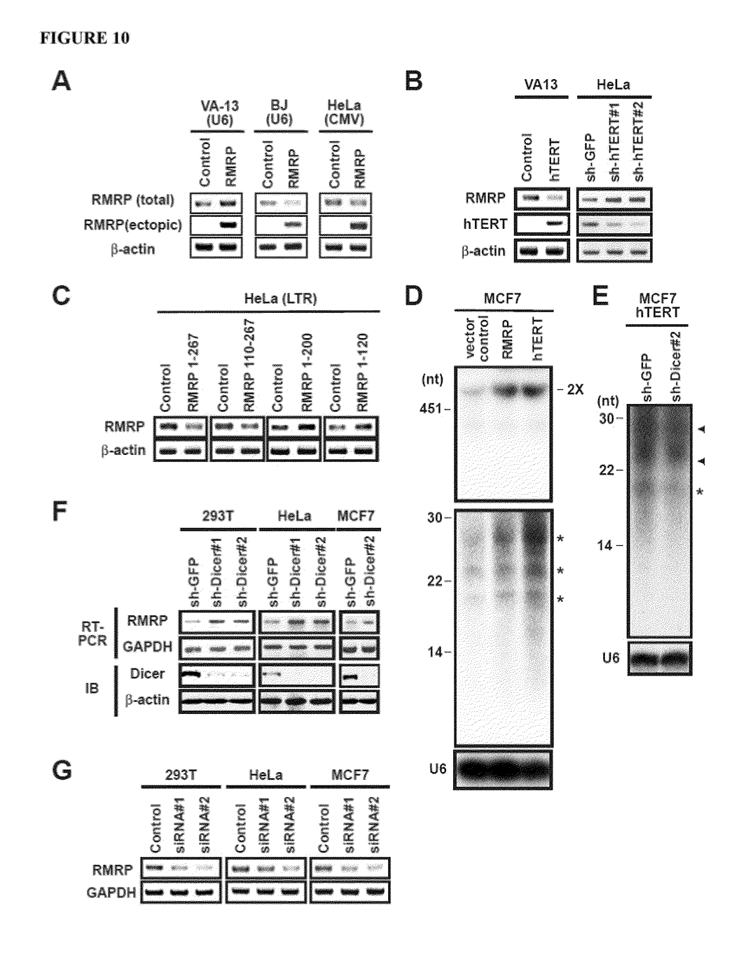

Upon expressing RMRP in cells lacking hTERT (VA-13), it was found that RMRP levels were increased (FIG. 10A). In contrast, in cells that express hTERT either transiently or constitutively, it was found that the steady state levels of RMRP were decreased when RMRP was overexpressed regardless of the promoter used to express RMRP ectopically (FIG. 10A, FIG. 15A (MCF7) and FIG. 15B (qRT-PCR). Forced expression of both hTERT and RMRP in VA-13 cells (that lack hTERT) or BJ cells induced suppression of RMRP expression (FIG. 10B left panel and FIG. 15C (BJ and qRT-PCR). Consistent with the view that hTERT-RMRP forms an RdRP, suppression of hTERT in HeLa cells (that constitutively express hTERT) led to increased RMRP expression (FIG. 10B right panel). Moreover, because the 3′ end of RMRP is essential for the priming process of the hTERT-RMRP RdRP (FIGS. 5E and F), the effects of expressing RMRP truncation mutants lacking 3′ ends were examined. As expected, it was discovered that truncation mutants lacking 3′ ends were readily overexpressed but failed to detect overexpression of truncation mutants possessing intact 3′ ends (FIG. 10C). These results demonstrate that RMRP expression levels are dependent on the hTERT-RMRP RdRP and indicate that RMRP levels are controlled by an RdRP-dependent, negative feedback control mechanism as has been previously reported in Arabidapsis (Gazzani, S. et al. Science 306, 1046 (2004)). To determine whether the observed suppression of endogenous RMRP was mediated by siRNAs produced by the hTERT-RMRP RdRP, Northern blotting with an RMRP probe on RNA derived from MCF7 cells expressing RMRP and hTERT (FIG. 10D) was performed. In cells overexpressing either RMRP or hTERT, increased levels of 2× template sized products (FIG. 10D upper panel) and small RNA molecules 19˜26 nt in length (FIG. 10D lower panel) were identified. We used sense and antisense probes corresponding to RMRP (nucleotides 21-40) in Northern blotting on RNA derived from Hela, 293T, MCF7 or THP1 cells. We found that these probes identified double stranded 22 nt RNAs (FIG. 16A and FIG. 18B).

Since prior work has shown that siRNAs contain 5′ monophosphates and 3′ hydroxyl groups (Schwarz, D. S. et al. Mol. Cell. 10, 537-548 (2002)., Schwarz, D. S. et al. Curr. Biol. 14, 787-791 (2004)., Vagin, V. V. et al. Science 313, 320-324 (2006).), we characterized the chemical nature of the ends of these small RNAs. After isolation from the indicated cells, small RNAs were treated with calf intestinal phosphatase (CIP) or polynucleotide kinase (PNK). We found that treatment with CIP slowed the migration of these short RNA species in polyacrylamide gel electrophoresis and subsequent incubation with PNK and ATP restored the original gel mobility of the short RNA species, indicating that the either 5′ or 3′ end of this small RNA is monophosphorylated (FIG. 16B) and data not shown). Moreover, we found that incubation with PNK in the absence of ATP did not alter the migration of this small RNA species (FIG. 16B) and that oxidation and β-elimination treatment increased the migration of this small RNA species (FIG. 16C), indicating that the 3′ end bears vicinal 2′,3′ diliydroxyls. Together, these observations confirm that these small RNA species contain 5′ monophosphate and 3′ hydroxyl groups and demonstrate that the small RNA species produced by the hTERT-RMRP RdRP are likely to be siRNAs based on their size and the chemical composition of their ends.

To demonstrate that the double-stranded RNAs produced by the hTERT-RMRP RdRP are processed into siRNA, we suppressed the expression of the ribonuclease III Dicer with two distinct Dicer-specific shRNAs. When we suppressed Dicer to levels that partially inhibited the processing of miR-16 (FIG. 10E, FIG. 25 and FIG. 27, we found similarly diminished levels of the small RNA species derived from RMRP but did not observe any change in RNase P expression (FIG. 10E, FIGS. 25 and 26A and FIG. 27). When we suppressed Dicer expression in HeLa, 293T or MCF7 cells, we found that the levels of endogenous RMRP increased up to 3.7 fold (FIG. 10F and FIG. 26A). Suppressing Dicer expression in VA-13 cells that lack hTERT did not affect the levels of ssRMRP RNA (FIG. 10F and FIG. 26A) but did result in increased levels of the elongated sense-1-antisense RMRP products of RMRP in cells that constitutively express hTERT (FIG. 28).

Moreover, we found that only the sense strand of these endogenous RMRP-specific siRNAs is associated with human Ago2 (FIG. 10D and FIG. 26C). These observations indicate that the endogenous RMRP-specific siRNAs are processed in RNA interference silencing complex (RISC)-dependent manner, similar to other small RNAs that are processed into siRNA.

To confirm that these small RNA species act as siRNA, we identified small RNAs from total RNA that hybridized to probes spanning RMRP, synthesized a siRNA corresponding to the identified sequences and tested whether introduction of a chemically synthesized double stranded RNA act as siRNAs. When introduced into HeLa cells, 293T cells and MCF7 cells, we found that this chemically synthesized siRNA induced suppression of endogenous RMRP levels (FIG. 10G and FIG. 26B). These observations provide evidence that similar to the RdRPs described in yeast and plants, the TERT-RMRP RdRP synthesizes double stranded RNA that serves as a precursor for processing into siRNA.

### Example 6

### RdRP and Heterochromatin Formation

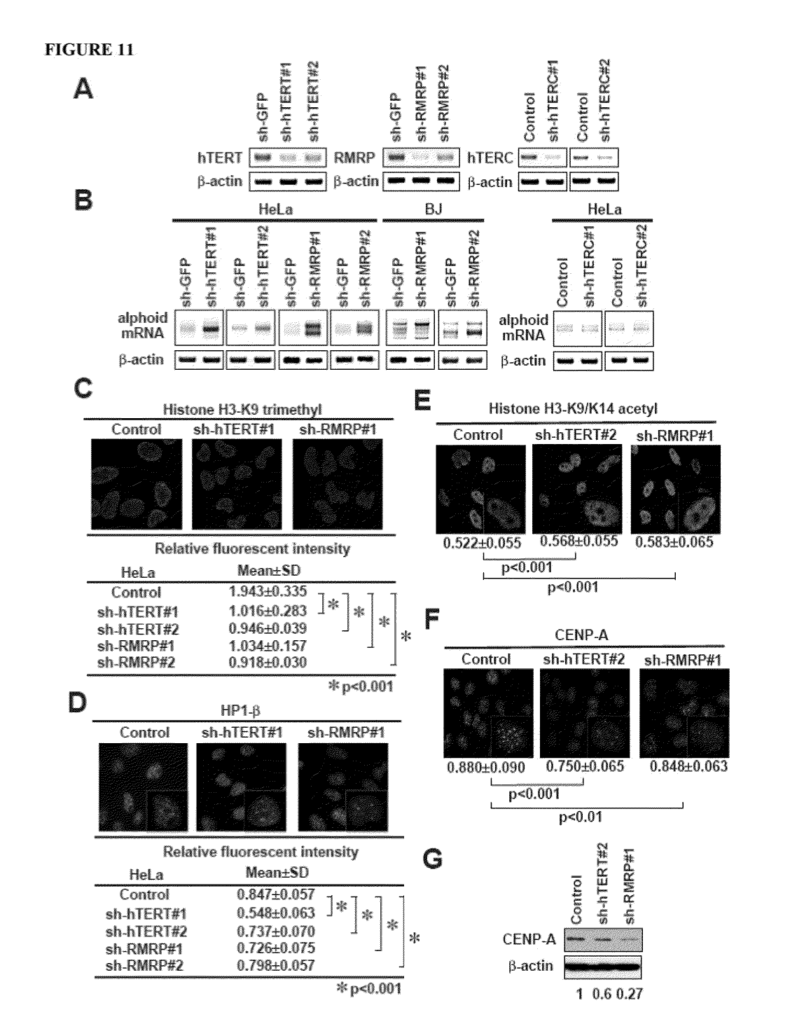

In fission yeast, inhibition of RdRP activity leads to loss of siRNAs that are associated with the RNA-induced transcriptional silencing (RITS) complex and correlates with loss of transcriptional silencing and heterochromatin at centromeres (Sugiyama, T. et al. Proc Natl Acad Sci USA 102, 152 (2005)). In addition, when RdRP activity is inhibited, siRNAs that are usually associated with the RITS complex are lost (Wassenegger, M. Cell 122, 13 (2005)). These results implicate RdRPs as a component of a loop coupling heterochromatin assembly to siRNA production. Suppression of hTERT in diploid human fibroblasts leads to alterations in heterochromatin throughout the genome (Masutomi, K. et al., Proc Natl Acad Sci USA 102, 8222 (2005)), To determine whether the hTERT-RMRP RdRP complex acts on mammalian heterochromatin, hTERT, RMRP or hTERC were suppressed in HeLa or BJ cells using 2 distinct shRNAs targeting each of these genes (FIG. 11A). When hTERT or RMRP expression was surpressed, it was discovered that the transcription of centromeric repeats was increased as measured by the abundance of alphoid mRNA (FIG. 11B), a locus that is normally tightly silenced in mammals (Morris. C. A. and Moazed, D. Cell 128, 647 (2007)). In contrast, when hTERC was suppressed, no increase in alphoid mRNA (FIG. 11B, right panel) was observed, indicating that these observed effects are specific for the suppression of the hTERT-RMRP complex.

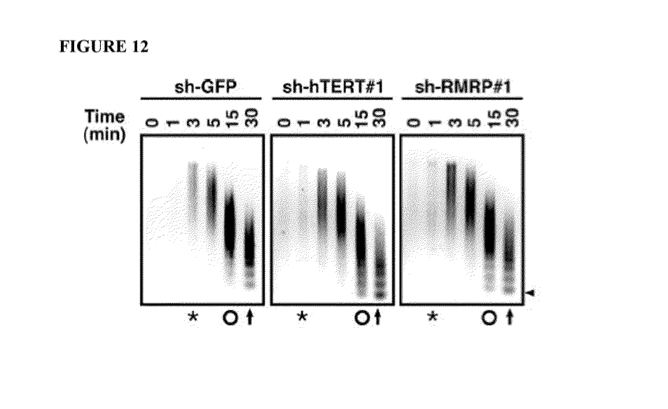

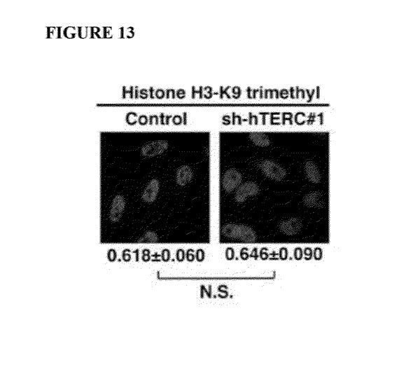

To confirm that suppression of the hTERT-RMRP RdRP alters heterochromatin throughout the genome, several measures of chromatin status were assessed in cells in which hTERT or RMRP were suppressed. Suppression of hTERT or RMRP rendered nuclear preparations more sensitive to micrococcal nuclease (FIG. 12). However, since micronucleus sensitivity is a relatively non-specific technique to measure chromatin structure, we then assessed several epigenetic marks that have previously been shown to correlate with the status of heterochromatin. Specifically, since siRNA production has been shown to be essential for H3-K9 methylation (Morris, C. A. and Moazed, D. Cell 128, 647 (2007)), an epigenetic mark that corresponds to heterochromatic regions of the genome, H3-K9 trimethylation status was monitored in cells in which hTERT or RMRP expression was suppressed. Significantly decreased levels of H3-K9 trimethylation was observed in cells in which hTERT or RMRP were suppressed compared to that observed in control cells (p value <0.001, FIG. 11C). Moreover, significant reduction of H3-K9 trimethylation status in cells lacking hTERC was not observed, indicating that the effect observed by suppressing hTERT or RMRP was independent of the effects of telomerase (hTERT-hTERC) on telomeres (FIG. 13). When we assessed other histone modifications such as HP1-β levels and histone H3 K9/K14 acetylation status (Grewal, S. I. and Moazed, D. Science 301, 798 (2003)), the HP1-β levels were decreased (FIG. 11D) while overall the overall level of histone H3 K9/K14 acetylation was significantly increased (FIG. 11E), Furthermore, RNAi-directed heterochromatin is required to establish CENP-A containing, chromatin at centromeres in fission yeast (Folco, H. D. et al. Science 319, 94 (2008)). When CENP-A levels were assessed in HeLa cells in which hTERT or RAMP were suppressed, the CENP-A signal and pr n were significantly decreased (FIGS. 11F and G). Taken together, these findings suggest that suppression of hTERT or RMRP expression modulates overall heterochromatin formation and link the hTERT-RMRP RdRP with the maintenance of mammalian heterochromatin as has been previously observed in fission yeast.

### Example 7

### A Mammalian RdRP

hTERT in complex with RMRP forms a mammalian nucleoprotein RdRP. Like those found in fission yeast, this mammalian RdRP produces double stranded RNAs that serve as substrates for the generation of endogenous siRNA, which, in turn, act to regulate heterochromatin. Unlike RdRPs previously characterized in many organisms (Makeyev, E V. and Bamford, D. H. Mol Cell 10, 1417 (2002); Sugiyama, T. et al, Proc Natl Acad Sci USA 102, 152 (2005); Aoki, K. et al. EMBO J. 26, 5007 (2007)), the hTERT-RMRP RdRP exhibits a strong preference for specific RNA templates, in particular, those that can form 3′ foldback structures, such as RMRP itself. Methods of the invention are used to determine the identities of the other RNAs that serve as templates for the hTERT-RMRP RdRP (FIG. 5G). Like other RdRPs, the hTERT-RMRP RdRP plays an essential role in regulating heterochromatin throughout the genome.

Since mutations in RMRP are found in CHH, these findings suggest that perturbation of the hTERT-RMRP complex is involved in the pathogenesis of this disorder. Intriguingly the involvement of hTERT in two syndromes characterized by stem cell failure (CHH and dyskeratosis congenita) suggests that hTERT containing RNPs play a critical role in stem cell biology (Calado, R. T. and Young, N. S., Blood 111, 4446 (2008)). Indeed, overexpression of mTERT in mice lacking mTERC leads to abnormal hair growth due to defects in normal hair follicle stem cell function. In mammals, TERT may thus regulate both telomere biology and heterochromatin structure through these two RNP distinct complexes.

### REFERENCES

Cohen, S. B. et al., Science 315, 1850 (2007).
Fu, D. and Collins, K. Mol Cell 28, 773 (2007).
Venteicher, A. S. et al. Cell 132, 945 (2008),
Weinrich, S. L. et al., Nat Genet. 17, 498 (1997).
Chan, S. W. and Blackburn, E. H. Oncogene 21, 553 (2002).
Shay, J. W. and Wright, W. E. J Pathol 211, 114 (2007),
Ruggero, D. et al., Science 299, 259 (2003).
Mochizuki, Y. et al. Proc Natl Acad Sci USA 101, 10756 (2004).
Calado, R. T. and Young, N. S. Blood 111, 4446 (2008).
Gonzalez-Suarez, E. et al., EMBO J. 20, 2619 (2001).
Artandi, S. E. et al., Proc Natl Acad Sci USA 99, 8191 (2002).
Sarin, K. Y. et al., Nature 436, 1048 (2005),
Blackburn, E. H. Nature 436, 922 (2005).
Masutomi, K. et al., Proc Natl Acad Sci USA 102, 8222 (2005).
Lee, J. et al., Oncogene (2008).
Ridanpaa, M. et al., Cell 104, 195 (2001).
Tollervey, D. and Kiss, T. Curr Opin Cell Biol 9, 337 (1997).
Moriarty, T. J. et al. Mol Cell Biol 22, 1253 (2002).
Lue, N. F. et al., Proc Natl Acad Sci USA 102, 9778 (2005).
Nakamura, T. M. et al., Science 277, 955 (1997).
Mourrain, P. et al., Cell 101, 533 (2000),
Nishikura, K. Cell 107, 415 (2001).
Makeyev, E. V. and Bamford, D. FL Mal Cell 10, 1417 (2002).
Du, T. and Zamore, P. ft Development 132, 4645 (2005).
Almeida, R. and Allshire, R. C. Trends Cell Biol 15, 251 (2005).
Semler, B. L. and Wimmer, E. Molecular Biology of Picornaviruses (AMS Press, Washington, D.C., 2002), pp. 255-67.
Behrens, S. E. et al. EMBO J. 15, 12 (1996).
Ackermann, M. and Padmanahhan, R. J Biol Chem 276, 39926 (2001),
Engelhardt, O. G. and Fodor, E. Rev Med Virol 16, 329 (2006).
Sugiyama, T. et al. Proc Natl Acad Sci USA 102, 152 (2005).
Masutomi, K. et al., Cell 114, 241 (2003).
Ford, L. P. et al., J Biol Chem 276, 32198 (2001).
Gazzani, S. et al. Science 306, 1046 (2004).
Wassenegger, M. Cell 122, 13 (2005).
Morris, C. A. and Moazed, D. Cell 128, 647 (2007).
Grewal, S. I. and Moazed, D. Science 301, 798 (2003).
Folco, H. D. et at. Science 319, 94 (2008).
Aoki, K. et al. EMBO J. 26, 5007 (2007)
Hahn, W. C. et al., Nature 400, 464 (1999).
Hahn, W. C. et al., Nat Med 5, 1164 (1999).
Masutomi, K. et al., Proc Natl Acad Sci USA 102, 8222 (2005).
Weinrich, S. L. et al., Nat Genet. 17, 498 (1997).
Kim, N. W. et al., Science 266, 2011 (1994),
Yamashita, T. et al., J Biol Chem 273, 15479 (1998).
Li, S. et al., Cancer Res 64, 4833 (2004).
O'Sullivan, J. N. et al., Nat Genet 32, 280 (2002).
McManus, K. J. and Hendzel, M. J. MeI Cell Biol 23, 7611 (2003).
Maida, Y. et al., J Pathol 210, 214 (2006).
McManus, K. J. et al, J Biol Chem 281, 8888 (2006).
Sakaue-Sawano, A. et al., Cell 132, 487 (2008).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference, All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### INDUSTRIAL APPLICABILITY

The compositions and methods of the invention are used to manipulate gene expression as a means to treat disease.

1. A complex comprising a telomerase catalytic subunit (TERT) polypeptide or fragment thereof and an RNA component of the mitochondrial RNA processing endoribonuclease (RMRP).

2. The complex of claim 1, wherein said TERT polypeptide is mammalian.

3. The complex of claim 2, wherein mammal is a human or a mouse.

4. The complex of claim 1, wherein said complex has RNA dependent RNA polymerase (RdRP) activity.

5. A complex comprising a telomerase catalytic subunit (TERT) polypeptide and a mammalian RNA, wherein said complex has RNA dependent RNA polymerase activity.

6. A composition comprising the complex according to claim 1.

7. A method for identifying an antagonist/inhibitor of the activity of the complex of claim 1, comprising:
(a) contacting the complex of claim 1 with a test compound; and
(b) determining whether said complex has RNA dependent RNA polymerase (RdRP) activity;
wherein a decrease of RdRP activity in the presence of the test compound compared to the absence of the test compound indicates said compound is an antagonist/inhibitor of the activity of the complex of claim 1.

8. A method for identifying an agonist of the activity of the complex of claim 1, comprising:
(a) contacting the complex of claim 1 with a test compound; and
(b) determining whether said complex has RNA dependent RNA polymerase (RdRP) activity;
wherein an increase of RdRP activity in the presence of the test compound compared to the absence of the test compound indicates said compound is an agonist of the activity of the complex of claim 1.

9. A method for identifying an enhancer of the TERT-RMRP interaction comprising:
(a) bringing into contact a TERT protein, a RMRP and a test compound under conditions where the TERT protein and the RMRP, in the absence of compound, are capable of forming a complex; and
(b) determining the amount of complex formation;
wherein an increase in the amount of complex formation in the presence of the test compound compared to the absence of the test compound indicates said compound is an enhancer of the TERT-RMRP interaction interaction.

10. A method for identifying an inhibitor of the TERT-RMRP interaction interaction comprising:
(a) bringing into contact a TERT protein, a RMRP and a test compound under conditions where the TERT protein and the RMRP, in the absence of compound, are capable of forming a complex; and
(b) determining the amount of complex formation
wherein a decrease in the amount of complex formation in the presence of the test compound compared to the absence of the test compound indicates said compound is an inhibitor of the TERT-RMRP interaction interaction.

11. A method of increasing gene silencing in a cell comprising overexpressing in said cell:
(a) a telomerase catalytic subunit (TERT) polypeptide;
(b) an RNA component of the mitochondrial RNA processing endoribonuclease (RMRP); or
(c) both.

12. A method of decreasing gene silencing in a cell comprising inhibiting or decreasing the expression in said cell:
(a) a telomerase catalytic subunit (TERT) polypeptide;
(b) an RNA component of the mitochondrial RNA processing endoribonuclease (RMRP); or
(c) both.

13. A method of treating a disease which is caused by undesired or overexpression of a gene comprising administering to a subject in need thereof the composition of claim 6 or a TERT polypeptide.

14. A method of treating a disease which is caused by inappropriate deactivation of a gene necessary for cell survival comprising administering to a subject in need thereof an inhibitor of the RNA polymerase (RdRP) activity of the composition of claim 6 or a TERT polypeptide.

15. A method of identifying an RNA molecule that forms a complex with a telomerase catalytic subunit (TERT) polypeptide wherein said complex has RNA polymerase (RdRP) activity comprising:
(a) contacting the TERT polypeptide with a test RNA molecule to form a complex;
(b) identifying a complex that has RdRP activity.

16. A kit comprising a catalytic subunit (TERT) polypeptide and a means for detecting RNA polymerase (RdRP) activity.

17. A compound identified according to the methods of claim 7.

18. A compound that increases the expression or activity of a telomerase catalytic subunit (TERT) polypeptide or an RNA component of the mitochondrial RNA processing endoribonuclease (RMRP).

19. A compound that decreases the expression or activity of a telomerase catalytic subunit (TERT) polypeptide or an RNA component of the mitochondrial RNA processing endoribonuclease (RMRP).

20. A drug or a diagnostic drug for in vivo or in vitro use for in post-transcriptional gene silencing or chromatin based gene silencing according to the methods of claim 7.

21. A device for the use in the methods of claim 7.

22. A method of treating or diagnosing a disease which is caused by the altered expression or function of an RMRP comprising administering to a subject in need thereof the composition of claim 6 or a TERT polypeptide.

23. A method of treating or diagnosing a disease which is caused by the altered expression or function of an RMRP comprising administering to a subject in need thereof an inhibitor of the RdRP activity of the composition of claim 6 or a TERT polypeptide.

24. The method of claim 22 wherein said disease is dwarfism, an immunodeficiency syndrome, asthma, atopy, an autoimmune disease, systemic lupus, erythematosus, rheumatoid arthritis, alopecia, aplastic anemia, lymphoma, leukemia, or a solid cancer.
